SEQUENCE LISTING (152 sequences)

SEQ ID NO: 1 - DNA, 20 bp, Artificial Sequence
  Antisense Oligonucleotide
tccgtcatcgctcctcaggg

SEQ ID NO: 2 - DNA, 20 bp, Artificial Sequence
  Antisense Oligonucleotide
gtgcgcgcgagcccgaaatc

SEQ ID NO: 3 - DNA, 20 bp, Artificial Sequence
  Antisense Oligonucleotide
atgcattctgcccccaagga

SEQ ID NO: 4 - DNA, 121434 bp, Homo sapiens
tatcctccctgttttctcacctgagagtgggtataatgaaatttacttctaagggttgttgtgaagattgaattagttagctaatttatgtaaagtacttaggatagtgcctggtatttagtaagttctatgtgttagcctattattattattactattaccatttaaaaatattatttcaaggatgggatgcattttctgtaaacatgttctcaaatgattctactactggatacaatactgggcttttatgcagtaattgttgttggaggagttgttctgacatccagcttgcacctgggctactggtttcgtggccagggatggccaggcctagcgtctctctgatcttgcacaatttatgcatagagtggtgtggttagaggaaacttggagagggggaacttctcagttttgctttcaggtatttatacattaactatcataagagatggacatcttaataacttccaagaaagagttcatgagcttcaatttgatatttattgtttgactaggtttctttctgtcttacctttccagttgttagggttcttgatatgtattttggtgatgaaaatttcaactctaaaagctaaacaatcaagatattgtaattatccagcaagtgattggcagatttcgagtagacttcttccattcttggctgaaatcaatgaaaatgtctactattgctgttgtttttgttcttttctctgaaaagtctgtttttgggagggcctcagatggtaaagtgcctttttattgtctcttaaagggaatgggggaatcaacagtttgagaatttttgttttatctgtatgtataaaattgccttctccttttatttcttttgatactctctctgcagtctctattcatgatctactccccatctttttatgggccccttaaagctagagattatgtttattcgtttatttatgtattccatttctccttttttctggatcctaattttgtgcctggtgcatactaggctctcaattttagtttgtcgaattactgttattgctgtttctgattacttcttgaagcggtctgttttaaaacttcacctgaaggctaagtaattcatcagtataatggtgatctattatttatctagcattttcctaaacactcattgatgaatccttataatactcatctgaagtatctaaatcccaagatgatgtgtcatgccatagatggtatggctttaatttttaattgggagatggagatgtaacagctggagatactaaaacacagagaaataagagcatcattcaagaccacaaacgtggttattgttggaagatagttttgggatgggcttctgtgctagaagccagaacataaaaaactgattaaaactctaaaaagaaacccagattaggtgggaatgaatgctgatctggattggattacacttctttggtgtctgagggttatgattagtgacttacttctaatgtgcatttgtttttatttttagcattgcagtgtcgagatggctatgaaccctgtgtaaatgaaggaatgtgtgttacctaccacaatggcacaggatactgcaagtaagtttttctcttcatatattttctttttgcgatagaacactggacaagatttgattctactcctctatttttaatgcttctgtggaatgttactggttcttgagctttcctggtacagattttggttgggggtgaggatggaaggatgtggatgccagataattggctaataagaacttcttactatcttactgttatctttctcactatgaaaaagacttttcagtgagtggctagttaatgtatatggggtagaccaggcatggtggctcatgctggtaatcccagcacttcaggaggctgtgttgggcgaatcacgaggtcaggagttcaagatcagcctggccaacatagtgaaaccctatctctactaaaaatacaaaaattagccaggtgtggtggcgcatgactggagtcccagctactcgggaggctgaggcaggagagtgacttgaacccaggaggcggaggttgcagtgagctgacaccacatcattgcactctagcctgggtgacagagcaagactccatctcaaaaagaaataaaaaaaaatataaaaaaatatatattagattcccttgtgtttttcagattaaggcatactcttagagtctccccttaacatgttacctattatcttgtttttaagtaaaaccaaatagatatcaactttaatattcggcccagtatatgtgaatattatattcatatttttttctagtgtaaaaaatctaattctgaaagtgagacagtgagagaggaagatgtgtgcatgtgtgtagattgagggggagcaggggctttgggtgtgtattattatatggggataaaagagtaaggatgaaggaaaatgtaggctagttcttggcttaagagcatttttttttcctttttctccaaccatctactctagccaagacgttttgtttgtttgaggaaggctgtgagataatttcataatagcttttctaggtttcctgcaaataatttttttttcttctgcctcaattgaaaatttttaagaggaatatgtatccattctttttatttcagggaagatgctggagaaataaaatttggtaatatgaaatttctcttcttttccttcatttttgtgaggagtagttgttctttgctttggtggaggtacttgcttagtaagcattttaagtgagtttatccaacacatttttatttcttaccaggaatgtaattacaacttttttccagtgagatctgttctgacaccaggatttagttttttaatgttataaacaagattttttttttcaagtcagaaataattttcttcactaaagtgaaaatattttaagctgtgatgacagtaaagcttaacaataggttgtttggattggaataaagataacattggaaataaaggttttatgtagcttattatgggctgctcatttagtttttctagctgggggaaaaaaaaacgtggtgcattctcctctaagaatggagatacaactggagataataagggagggaacttaataccttagagtaggccactgaaatcttgtttagtctttttgtggcatttggtgccttagttgcttgctttattctgttatgcaactcttgtggtagttaaccccattgcattatggtcatttgttgatgtgtttcttttgctagaatgtgaggtcttagtcttatgcattttttgcatagttaatgcctagtacagtgcctggtgtagttattgttcagcaatgtgtttcttaaactaaaaggtgctaagtagataccatcatgcacatgtttcttatctatttatgttaaataagaagacactggttctctcctttaaaaatttcagtgtggcgattcctcaaggatctagaaccagaaataccatttggcccagcaatcccattactgagtatatacccaaaggattataaatcattctactataaggacacatgcacatgtgtgtttactgcagcactatttacagtaggaaagacttggaaccaacccaaatgcccaacggtgatagactggataaagaaaatgtggcatgtatacatgatggaacactatacagccataaaaaagaatgagttcatgtcctttgcagggacatggatgaagctggaagccatcattctcagcaaactaacacaggaacagaaaaccaaacacggcatgttctcactcataagtgggagttgaacaatgagaacacatggacaaagggaggggaacatcacacactggggcctgtcagggggtggggggaaaggggagggagagcattaggatgaatacctaatgcacgtggggcttaaaacctggatgacgggttgataggtgcagcaaaccaccatggcacatgtatacctatgaaacaaacctgcacattctgcacatgtatcccaaaacttaaaagtaaaatttaaaaaaaatttatttacttcacatagcattcagggtggtaataccatacagagaggcaaattggttctatttgaagatcattcaacttggtgacttatatatggagaatttcaaatatgttctaagtgttcgcctatatatttttggtctgacatggggaaagaaggtttctaggggagactaatctctgagcacttaccaggagaggacacctaagggtcactgttgggaagaagtccaatagttctttgttggactgttgggctgctagtctcatactctccaaccaaggttggagaccatcctggctaacacggtgaaatcccatctctactaaaaatgcaaaaaaattagctgggcgtggtggcacgcacctgtaatcccagttactcgggaggctgaggcaggagaatggcctgaacccaggaggcggagcttgcagtgagccaagattgcaccactgcactccagcctgagcgacagagcgagactgtgtctccaaaaaaaaaaaaaaaaaagaaaatggagctgcctaaattctgtccctggctacttgttaattgttgataggatggaatgcttatggatacagttctgggtatgtccctcagatgtctttcctgttttgagggaaacaaactccttgagagagagtaagaaaaaatttggtgaggccatccttttgggagaccaagatgaacaacaaaacttgtataaagagagtgattccctacctcagcaaaagtaatattagcatatttaggagtggcagggtcaacataagtggacaaagaaaaagcaagaacaatatcaaggcagccatgtcaatcattctacagtgctttgccagcctccacagaattttaagcaaaattttatgcaaaaataatgtgtacctagcagagctttcagtgtattttcaccaaccttacctttttttctgtggagaaagaagatgatctagcttgttcagtaaatattattttcacaaaatagaaaataaatattaaaacacttagaagatttataaaaattctatcaggcatgcagggcatgtgattatctttttattcatgcgattgcttagtggattaagaaaagaaagttgaagccaaattccttttacagggccagcgtaggggtgagtggtgagtggtgggatgggaactggtagaaagtgttttaaaatagaaggaatctgactatctgtttagacagcataatgctactgattcagctaaaatataatgattgtttatagaaattataaaaatccttgataagatcacattgacagatgaagataactacttttgaagattagttttgtactcagctccactgttttttctcactgttaggagcctaagtaccctatcagtctgtgattagattttgatgccagattaggagaacgtgtataatcttgcataaaactcaattattgctgttaaataactactgtcaaatcttaataaagctgtctcactacagggagataagatattttattctttcccttgtcctactgggaggtgtaaatatgtaattaaatttgatgtcattaatcactttcaagttgtttgcttcagactttcaaatataaaccattctaaatcccaatttgattaaagatcatggacaactcaagttcactaggattctgggggtaggttaggttatgatttgtataattgacaaaatatgtattttaaggacatttattgtagttatttaaaatctttgtctgctggttcttaacatctgggttgtctatggatgtgtttctgtttctatcaactgctccttctttggaccatgggtatgacttccctttactttacacatgtatggtagttttacattacttactggacattgtgggtgatagattttaaagtttatctcccactaaagaatgttgaattttgtcaagattggcagatcatcttgatcctatggatgctaggttttaggacacgttagggtgggcctacttctgttctgcccttagtccgatgacacaaatcttagtcatgggatagggttcttattcccaagatgtgacccttctaggggttcagtggtaagtgtgaggggcctaccaagccctgaggtgttcattaagcccctctaaggcagaacttgtcctcccagccccaggagccttctgctcagtcttttcagccgtccagctcttactttctcttgggtttctgaatcttatcttgctcatgcatagtcaggaattatttaaggatttgagcagagttgtatgcatactttgaagctacccactctgtggttctgtcttttccaggaatttttctcctcatattctagctgctgtgacagctccagatacctctgactcctcagtatcacaggacttccattttctgcttgcacactcttcctcttctgctgcatgaactggggtgtaccttcacgataaaaaaaaaaacaaaactgtaaatgtgggtttcatatagtttgctttgctttttctgaggcttatatcccttctagtttatgcctgctttcagtgattctccagtgcctttgatattttgtccagagcaagagggctagtctgatataagttactctgcaattatttttaatgatgaaaattggaaatataaaggggcctttcagaatttcatacataccacttggtaaatatctaacgtagtgtttgtaaaacagcagaaagtcactgtgagttgtatacattcctgtagatgggtgcttgcatttgagaatgtccactttttgctaaagtgctgatctaaaaactacactttaaggtaaattattataaatataaagtgttataaaatataactttgtgtcacattttgtctaaaacttgtctgctagctgtaataaacattaaacattaacagctaacaattttccttgccaggattaggcagtcatgttggtggtccagatttcctgaatccatccaagaaaaactagagccattgccttctttgtcttcttggtaaatgtctgtatagtaagtagagagtagagacactcataatcccttagaacttagctttttatggaggatgcattcgcaatggttctgttgtggtgcttctcccccaaaagggcgttttcagaagtcatggctgactcagcatttccccttttccttcaccttcttgtgagtgcattcagggaggcacaggggcattgtcagatcaaagaaatagacagggaaaaatatcaactgttaaattactttctctttcttctcagctgaaactggttctgccagcctagttccttcaagtacggtgcctatttagctgaacattgtgtatatagtttttctggaaaatgatagcagaagcttggccagttttccatagttcatttatctttttaacataaaacaaagaatgctgttttcttggctcattaaatacctctcatagagtaatcttttcttaaaatgtaatgtgttcaggttttattaattcaagagttatttattgcatgctatttatgagtaccacatataacagataactcaagtctgtggtagtagttgcagttaatcaatattgttatcatattatttcacacttctttgggaagtcataaaataattaccagttcattaacttgggcttcttagagaggttcagagtaaagccaagtatgaaaaaaactgactataatgtggccttctgtacaggggccgccagtgaatggcagggctgggaccatggtgaggcaagtaaggcacaggcctctcttgtgaaatttaagagagtgccaaaaaactcactaatcaaaataatgtttgtatgtagttgttcaaaaaatcaaaattaatgcaaaataagttgaaaattatcaaaattttaaataaggagataagtactactgattttttcttttgccttatgatctagtatggttcagcctgcactggtgagtggagtaagggtcctggaagatccttgactggtgacatgaagaggacatagttttttgttgttgtttttttttgagacagagtctcgctctgtggcccaggctggagtgcagtggcgctttcttggctcactgtaagctccgcctcccgggttcttgccattctcctgcctcagcctctcaagtagccgggactacaagcgcccgccaccacgcccggctaattttttgtatttttagtagagacggggtttcactgtgttagccaggatggtctctatctcctgacctcatgatctgcccgcctcggcctcccaaagtgctgggattacaggcgtgagccaccacacccagctgaggacataggtttttttatgccagatagacttaagtttgagtcctgattcctattaacaattggatctttggcaagttaaagtctataatgctagctaacattattgcgtattagggtctgttctcaagtagtttaacatgtatttactttagtccccatgtgagagagagactatatggaagctaagagaagttaaataattcactcaagatcccatcactctgtagctaacctgagatatactcgtggaaaatgtactgtcaccttgagtctcagtttgcctgtcaataaaatgggacagaattacctgttttaagtttgtagttcaaatggaaccttctgaaaaggaagtgtggaacatttgaaagtatacaaaaaggggacttttcagatcaggaatatgattaaaacactgctttatgtaatttctaaatttaaagacagtagcattaggtaggcctgagtacctcatatcattctagaacactaaatctgcatatagttacatgagtgtacatgggccaggtttggtgaatactgcatttctgagaaatggcctttaacatgaaagctcaatgtaataactgtggatgaaatagaagaagagggcagtgaaggtggggatagaggatattgtctattaaagtgtgcttcagtagagagaactaatttgaaacttctcctttctttttactaatggaacaggagcaagtccagacagatttatgcagttgtgaaattgagacaggttaaattcagtgatttttgataaatgttgttgccagcacaaaagtcacaagttgttgaatatggcaccttacatttgcatagtgagttacagatgcatatgagtatgtcctgtgaggcttttccttgtattaggacttgagatctggaccctttaacagtaccattcctaagtgattcttattaatacttatgtcctattaaaatcctttatcaaatatgatgtttccctaaggggtcttttgtaaaggaagtcttggttactagcatctccatctgtggcccaggcttgagtgcagtagagcaatcatggatcactgcagcctccacctcctaggctcaagccatccatcctcctgcctcagtctcccatttttaaaactttttatagagacacggtctcacatgttgctcaggctggtctcgaattcctgggctcaagtgatcctcccacttctgcctcccaaagggctggaattacaggcctgaaccactgtgcctggccagttagtagcttcttaacacatagcaacataatttccatactattttcaactaatttttattctttcagttgggacctttatatatttcagtgttcagtgaagaagtaggagaatctggttcttaggctctgcctatatccttttacctgtcttgggtaactgccaagaaattaaatcaacaaattttggcctgcagtctggcattaatagatgtgaatttagctaggtttctttcaacacagtctccaggtcaatcgtaatcttatgtgaagtttcttattcaggcatgtctcattagcaccattaatttcacacaggaattctttttttctccctttttttgtgactaagcaaaatgagaaatatttctctttcaggatatggagaggcttaattaaattctttaattttgtctacatctgcccttatgaatgatagcaaaggaatgggggatatcagaatatgagaaaaagtaatcatctaaaaatttccaagcactttaaaatttgttaagctagcataaatatgtgggtgtgatagaggttttcattaggcctcctctcaacatacagtagttaggaatgaagtttagtgtctctccgtcctcctgtcatatgtgtgtatgcacacatgtgtgcacatgcgactgtgttatgcattttgttttcatatttcttttagttgagttttaccttacaacccaacgtaaggtaaattaaactttctattggatttccgcacattttctagatcctatttattcactgtcagtgaccttcactagcacttagtcctctgtgtccaaaaatgtattgccaattcttagactccatcagccttctcctatgaaaaagtaatgcagttaatttctgctcgaaatgctcttctcctaaacagtcttctacttgatctttatgaaaagagttttatgaaagaaaagtgaaaataaagactaagaaaggcattatttaagtgatgggtgatattattcagcgaacacattcaactagagcattctatccagttttttgccttcacgtaaatatgaggagaaaggtaggtgttaataagaaaataatctgattaggatttatgtagaatggtagcatttagactattaaatctgtatagattcaggtcattttcctgcagtcactgtctataatcctgtgtgctgaacactgaaagttagatggactctgaagcactatagaatcctcacttttcaaacaaaagtttaatttttatcacactagtatttattgagcaggctactataactgatcactgtactgggtattgtgaatacccagaaaagcccaagaggcctgtgttacctgacttccttattcacaagtatacgtacccagagactgtctgacagtgaatgcagtttctcctgcataagacatggtggcagtcttctgaagtcttgctggggagatgaagcatacagagatgtgtaaaataaaacagtttgtaatatcccagtgtaggggcacagataataactatggacactcagacaaaggaacaagtactgtgggacaaaagagtaaaatcagaggacttttggtgcactgcttagatcctaaatctttgaatattagtggtgccttagaagacggataggtaggagaagggtgagaggaagggagctgaggagactagaagggactgttatttcctggaataatcattaccaatttcttcatcacattttcttaatgacacagtttcaaacgttttcttcatcttgattgctgtcttctgtgtatatgctggtttgtgtgaggttttactaaaccagtatgtggattaatctagtttcacagtgacaggtaagtgatttagaaaatccttcctttagcacatgttaacttgaacgtttggaggtagaatgcctagagtgagggtgaaggagaggacctggggacaggaagagagggcagctgggagcaattatgctccttccctcagacctttttggccttgactcttgattcttgtacttctaccagacagagaggcggtaggcttgtggccatgttttgcgaggaaaatcagaatctaagaatctctaaggcaagagcaagaatggattgggatagggaaagtgataggaaaacagaggcactaagaagagggttggttttcatgtactgcatagttcattcccttagttgagaaatcaggtatctcaacctcagatatcttctgtccagcatatcgctggccagatctgattcatatgagggatccagagtaaggaagcatctgttagccaagtctttggttaccccaaattagctcaggtcctttactgcttgtttcctctgattatatagtacatatccaaaccctcaattttccagaataaactaggtctgcctattctgtgtggaaaacatttgatggtggttaagattctcttgcctagacgatggtagtgccttagtcgtttaactgcctgtcactttaagattttgaaatactggttctcgttgtgtaagccagacacatcttgagaatattttacggggcaacttgccctttctgaaacctcagtcatcctgaaaagtaactctgtacctccttgactttaatttttctgtcttaattctatttgtatacaatgtttgtggtgtctgattctctgtctcgtattgctgaaactcatggccttgaatttctgggtcactcttaaaaaatagacataacttgattactaatgaaaaagtgactcattttcttccaggcaaaggctgacactgactgtcacagtttgtgacagaagcaaactctctaattaggagacatcacctattcttttctaaattttataagaattgatgatagcatcatgagatgggaagtgagtaagataagcgtttatgcttgtagagaggatgcataaaaatgaaatctggataaagttctatctgaatttaccattttaagtggttgattccctgtgagcttaaatttagtgcttctcttcccagaccgttgtcttacatacacacttcagtcagttttagccttgtataactaatcagcaggtcagattccatagaggctgaaatacctaccaccattctatgctcaagaaaacaggctctcatgttggccagttgtttgggatagaagatggttagacctttctatcccttttaaacacaagattctttttcattccccttatcttatttaaatatattgttccttctgtttggttgttctttgctttcttatgcatctgtgaggggctagctgaaagtgtgagttgtggaacaattcatttacataagtgtatattgaattatctgtgcacctagagggtgatgggcactgtgagggatattaaaaaagagacacagtacagttcttaccctaggatcccctgcttggagtgggacagtggtcaagacatatacaaccacaaacacccaaacattaacatcagcaagcatcattacatccttgggtggtattgccaagggacttcaaaggccaagattatagagtcatcacacttaggaggtcaaagggactttggaaagcacctttgtcactcattttgttttataagtgaggcggctgaggtgtaatacagctaagcgaattagcctttattacataactcattagaatgtagactgggactaagagctgtttctccagatcccattaggcaatctaaagattctgcactatttctttttcatttttggtatccaaataggtacagttgtttttgatctaggaaaatccttttagactaaacagtaagctttttaaattactctgcttttagatattttttggacttcagaactttgaaggtggtcatccaccaacattggggaatggaaagaaggctccaacacttttctttctagagatcttctttaattttcaatggcagtcactacagaatctagcagttcatgctattgaggccactttgtaggatttttttttttcttagtcagggctccaggtccctggtggtttatgtagtcagtcttttatcaacaaccgatgaaagactaataaaagtgcaagagggaatgaggactgtgtgtgtgtatgtctgcacatgcacacatgcctctctgtgtgtgttggttaacttcatatttggtaggtgggagggaaggtgattaaaaaataatctactttgcatggagttggcccaatttgactgatataggggtgagtgggggttaagaagtagtctatatatcaggaatcttaattagttttgaaaatcctttaggtaaatagctcaacccagttgttcccaaacttcaagtgtatttatagggtctttttaaggggtaaagattccacagagcccttcattttgaccttgtgcttctgcactgaatagttgaatgccttctatatgccagtcattgtgttaggcactgggagtatgagaattaataattttagtctgttgacagataatcataataagggctaatgagtaacacaggctcagggttataagaacagagggggtatcaaggctgtttttcacaggaaataacatttaagttgagacttaatgaaattttagggcttagccatgagaaagtggggaaaagcattttgggctgactgaacagcgtgtacagaggtctggaagtgagaagtagtgtactatagcattttctaggagctgaaaggcgtttagcaagagtatagtgcataaaggagagagcttgagatgaaacaatttctcagtgtgttgtttgaatacaatgtatactgtcaaaatctaagaattcaagaatcatttaatatatctttatgttttactagtcataaaagcatatacatatatttaaatatggacaagatactgtttagtagtttagaaagtgatttataaggattgcaatcccttgcaataactccacagcccataccttgggaacaactgagctaactcattcagttttgtccattacatcgaagggtgttttttgtaaactcatatgtctagaatgtgtttgccatgttttaatcacaagtagatattttctcagggtccagtgcagtgaagtatggtaagatcctgattgtgttctggaacacagaggaaagaccaccttctgttatagcaacaacacaagtcttttaacactgtgtgccccttcccaatctttcaagtgatgattgaagagactaggtgctcagctcagcctttgagttctgataaatgagcccagactgtaaactggaagataaggatgtttgtaaagttcttgtataaataaagcatggtttctcattgcagtggttactgatttcatagtctgagtgaagatgaatgatgctgtgaatcaacagctttaaagtccgtatcacttcagcttctttttggtttaggtttcttaaaatcagtgtgtatttaatgctttattcagatgagggggtgaaaaacctaacacatgtaaactaagtgaggtggggtttcagagataattcccagcctcacaattcctcgtgaagttcttttcctgtgggaaacttttaatttggaagcatgcaacctaatgtgggaaccaagattaacattttctgaaatacttctacaagaaaagcagaaatggtctgtccaggaagctgaatttacatagtagaaaaatgagctgccctgcagtatttggtagtctttgtgtattagttgtgataaaagtgtgtatgtgtgtgtgtacgtgtgtgagagtgagagattgtatacttgtctttgtttccttcacatacaactagtaaggccctagaaaaactacactagaaagtgtgttttaccacaagcgtcccagttctggacaccaatctatacacaaatacttttttttaaagttctttttgtttttccttcttgctgagtaagctatagtatttcctttttttctttcttttttttttgagagaagggggggttgagagtagagtgggaatggcaagaagtagtatgacagagcttcttctctttttttcccctctttaccaggaagttaactagaagtcctcatgcatgtttttaaaacaaagttggtaattagcataacctagttagttacctttacacagagtgacagaattaaaaagttgacaagcccatcagacctcagccaggaggtactgaaaggagggagaccagtgagtttagaccaataggtgggttaggcctcctgaatgccagcctagaagtttagacttgattctataggctctggggtacctacaagtttgtagtcggagccttgggaattgaatgttacataggaactttcactggttccagctagccttggctgttagcaattatttttatctactttaacaggggggacagagtaggggggcaggaaactaagctggcattatggtcacaggaaagaacagactgatttggagcctttcaaactgcagacctttgttactgaccgatgcttaatttggtttctgggttttgttagttttttcccctgcccttacctcatttaccttaacgacagctcccccctctagagctcagctagggcaggctgccactgcggattggggggccaagaggcccagcgcaagaagaaagtgggttgaaagcagagttctgttcaaagaattttctgctggaaactagcccagagggagtaaagaggagctttaatgaggagcagctgcagtgccgacgcaacccacatgagactttttttccccttcgttccacattctgtatagtttttttaaaaatcatgattttgaaatagctgttttgtaaagcatgcctctctttttcttcttgtatgtggtgggattttgctttgttgttgttgttgtttttttttgaatggccaaatcctcgtttttaaaaaaaaaaaaaaaaaagctaaagacagagctgcagcaaagccctggatgcaatttggcctcaccctgctgatacagaacattcggtggagaaaacaaggggagagaacactggcttttatttggaaaaggggcttatttcctgctcagacttcagtcatcttggagctgacacaagctgctacactgttttaagcttttctgtagacgagtggctattcacttaggaaacgtgaaagaacaaatttttctgtcctgtattactagggagactgatcctgaactgcagccattgccagatagattggaattgctattcagatcccagcttcgttgaaatctgtaaagtggctacatgtaaactaatccaggctgctagtgagatgtgagggttggggtctggttttcatctgcttaagtgagaggaaactgtaggggtatccttcaaatggaatgttttctagttccattaggaggaatcccttgtttttctctgttttcttccttcctttgcttttctacatccaaccccatgcgtatgtttgaacagtaacaatggaaatgtggacctctcaatgtcagaaatgacacttttttttttttgagatggggttctcaccatgttgcccgtgttgtctaggcaggtctcgaacttctgctctagcaatctgcttgccttggcctcccacagtgctgggattacaggtgtgagccactgtgcccagctgagacttttaaagaaaaagtcataaatacatctttatggacctggtaagtattcagattagtttatggagtttaaaatggaaaaatgctccccaaacctttctctgtattcacaacctcatctgtgaagtatgttctttcagagttcaatgctagagaagagagagtaagtaaaagtcactgagagacattgaagagacagttatgaaaataattaataaactccctacaggaggaaaacattcatatagttttgcggatgaaaggcatctgttagatttttgtggttattttttagaatatacacctttagttcaagtggtagactattttccaaaatgtgtgctgggtagcttaatggaattatagatatgtaagggttgttgggtgtttagccatgttttcactctctaaccaaattcaactttagccatctcaagaagacagctatttaagcccttgttttcaagcagagaaccttactttccaccaagactaacagagtatattttctccttataccacagttgaacagggatttgcaagttgaaagatctcctgccttaggaaacaagggtctgttatttctgactgtcttacctattactcccctttcttcccatgtccataatgctgcttagcttcctggggtgtgaaggataattctgtacctcaggatgactattaggttgatttggcctgttattctaccagacagatcacagagtagagatggattttgtggaacatttggattgagagactgactttatccatcacatgaaatttagcctgctttggaaccagctgcttagtggggtttaggtactcaggtattcagggaacctacaccaagggttcaagacaggctcttccccacaaaaactatctagatgggagtttgagaaaggtacagcaaagacataacagtggcttaaataaagaagaaccataccagtggagacaatgtagtgcatgacatggcactacatctgctgttggaaggatcagagataggtgggatgatttgtagaaggcttactataggacaggtttacagcagatttttgaaaaaagggtggaagaaaggaacctaagttggagatataccagaagggaagcatgatgtttctcactggaaattaaaaagattttaatataggggacagtaaacaagattgtggaaataatatggagtgttcgagttatggattactaaagagtaagatggttagatgattggaatgggacagacaggcagatcagagattgtaaagaacttggatttgaaaggcttggtattaatactgttgctagtttctgagcagaagaatgacatgaaaattatgcttaggaaagattattcttttagtcatatgcttaggttcagcaaggaatttccagactgtccttagggagaggagaggtatggataggatgctggtccatttaggatgttagagctgattgaataggtgctagtcttggggcaagacttagtcatacactttcagttaagtactatttcattgcaaggtctaggtgaagatagggatatatttgtaaaactggggaaattgctagattcacagataaccaactttgggcaaatcagttagctttggtgtccttttcagtgctataggggttcagaccatttggtttctgagatcactgctgaaattctgtgattgtatgagtataatgtaaaagttgtctcaatcaaggaaacttggagtaaagactcaatatgatggatttaaaaggaatgaatgacagttccaattattgggttcaaaaaacatgtgctacaagtataagatgagaaaaatagggtacactagtagttcatgtgaaataaggctctgaatattttagctgagtgtgtattccatagcacatagtagtattttgtggcttctaaaaaagtacacatatccattgggtatgagtcggagcattattctttcttgaactgcgctctcacgatgcctcctcagtttaagaacttactctgaaatagtaaaagcatagtgtccagatcaggaggtcatggttcagctctgtgctgcactggtaagaacatgtgttaagcctttttccattttgagcctcactttttaaagaggtttttgatgaactggagtcatccagtgatggggaggcaggatagcctagtgttgaagcacacgcactctagagtctacttgggtttgaatcctgcctcttccatctactagctgtgaatttttttttttttttttttttgagagggagtctcgctctgtcgcccaggctggagtgcagtggcgcgatcttggctcactgcaacctccaacttctgggttcatgccattctcctgcctcagcctcccgagtagctgggactacaggcgcccgccaccacgcctggctaattttttatatttttagtagagacagggtttcactgtgttagccaggatggtctcgatctcctgacctggtgatctgcctgcctcggtctcccaaagtgctgagattacaggtgtgagccaccgcgcccggcctagctgtaaaatcttaatctttctaagcttcgagttcctcatatataaaatggagattataatatagcactggagcgagtaatacaagagacaatagctatcaaatgcttagcaaatatacaatacatagttaagtacttagaaaatatgttttcagttacttcgggtatatacctgggaagggaggcagaaaattttggtcattctgattattatgactactattaagcatagaagcaccactaaaaaggtgaagaaacctggaaataaccctatactgaggaatacgccaaaccattgaatcctggaaaaaagaaaacaaaaggcccacataactgtcttcagattcttgaagggctacgtgtaagagaagcagaaggcttgttcttttttgttctagagggtagagataacctggaaattgtagagagacaaagtttgttcttttcaaagttaagacataatttatgtatcataaaatttacccttttaaagtgtttaatttagtagactttagtatttccagagttatgagaccaccaccattatctaatttcgaaacatttttatcaccctgaaagaaattctctactgcttagtagtcactgtctatttccctttctttacagctcctggcaacactaatctactttctatctctacggttttgcatattctggacatttcatataaatggaattatataatatgtggccttttgtgtgtggcttctttcacttagcatgttttcaaggttcatgcatgttgtagcatgtatcagaatgtcattccttttcatggccacatatttcatttatggatataccacattttatttatctgttcatcagcttataggcatttgggttgtttctactttttagctattatgaataatgctgctatgaacatttttattatctgggcatatgttttcagttatctcgggtatatacctaggaagggaggcagaatttgaagaattaagaaagaaagagctttctcacagtgggatccacccacattgaagactgttcttttgttgaagtagtcagccagtaatttcacttaacaaatgcatagtgtttactatgtgctggatagtgttctaagtgctttacccttatttaaaaatcctatgaagtaggcactgttattacatctcttttacaacagggaagtttagtaccttgcccaagattacacattgagtaaatggtggagttgggattgaaatccaggtggtctggttccagagtctgtactcttaagagcagaacaataataactccaagcatttattaaattcttaagatttatccggcaccatgctacatgcttgtattcaagcagaggctggtgatcatcttttagaaatattttagaaatgattacttaattggatagaaattaaatttcttgaatctgaaggttctgttattccaaggatggctggaaatggaggaaaagccagcaacaagatgatgtctggcctgaggcttttaagatgatcagaatttgtatcaaaaaggaaactgtgaacacgaatgataagtattataggaattcagttaacaagtcttgtcagaatgtggtaataaattatatacaacttaaagagatgaagttactttaaattgtgtgtggaagccttagaaaaattgaataattaaagtttcttgggaagatgatctcctcttacaaatgtttagtttcaaatagtggcagcacatattaggatagagagatgacttctagaaaactgaacatatgggactggaacagtgtattttggagtcagttacaggaaggaagttgtttaaaaccaagtgaacaacaacagtaacaaatgcatttgagagaaagagcagtgcagtccagacttaaacattagggtggccataatagatggggaagaaggagaaatcagcaatccagactaagacttcactgggttttgagttagacagagggagtcatagaagccaaggtaaaatgattaaaacggtgcgtggagtggttagcaattctccctataggagagagaatttccttatgaagtttcagatgagaccactgaacttgactagaggaaagtcattgtttttagaaaacagtttctaagcaataattagaattaaagttttgttgcggagggataagtaagaagtagatggaaaagaaagagaaatatctgaaattaggctacagtcatagaaggttagctgtgaaacaataatttggaaagggaaatgggacctcatgaaagtgtgggttaaaagagacttgtatatcttctaaggtaaaagtaaagaactaagctgatgtccagccctctaaaacattttaaatagaaatcaaactttattaatacagtaggtctagtttcacataaagcaaatatatggttctgtacatgggctttaggagagtaagtctgttgtacctcatatgtaagtattatcccatttagaggaaaaccctaagtctttctttacagtggcttaaaggccccaattgatccacatcccagttacagctctcatctattttctgctgctccagatattcttgctgtttctcagacacgctagggctgctctctgtgttatggcctttgcatttgctgttccctttctcatgaatgtgtttccctaagatacctgcatgattactctcaccttatttaggtctcaactcaagtattgtcagtgaaggtcttctctgattttcctattctaaactgagatacttctctttgctcaccccacaccctcatccccagctccattccttcctgtttaattttttttcacagcacttaactccatctattgtgcatcttttacttagtaattttgttttcgtcttcagctattagaatgtaaactccaagagtgtaagaatttttctagcactggaaaaatgcctggcacataataggcatttactaattgttgtgtgaatgaatgattataagtttaatattctttgtggaaactaataaggttgatttcctatcagaaaccgatcattattaataagcatattggatggtatttatagaattagccttcatatgttctctactcagaagttggcaaactttttctgtaaagtgccagagagtaattatttttgcaggctttgtggtctcattacagctacttgactctgttgttgtagtggaaaatcagccatagacaatctataaatgaatgagcatggctgtgttccagtaagactatttatggacactgaaatttgaattgtatataattttgaaatgtcagaaaatatccttcttttgttctttttttctaatcatttaaaaatgtaaaaaactattcacaggccaaacaaaaacaggcagtggtccaaattagacttgcaaactgtactttgtcaatctctggttttactgatttcttaaatatatcttaggttaaaacttatttttccatacttgatagggagcaacaatgtgaattaaactgtagaaacaaataattcagaaatacatctgtttagctttagaaacttccattttgcttcatttttgttttcctgggaaccttatatttccattatatgtttatgatgctaccatttaaatgactttgcattctcttagcatatgccacatgatagtgttagggtggtgtaatagttcaggagcaaagctgagcaaagcccacccacgtttaaaaatttgccattatttagaaggtgcataattcccatatgggagaatgaagagtcatctaataatgcccagagggctgcactgtgagtgagaattgtgttgcagtttttaacattcataacacatttcttgaataagctgcaaatgcttttaataatatatctcagagttaattttagctatgctacattggcactttttgctcctccacttcaaaaaaccaacacaagaaactattctcaaatcaagctgtgatttggatggggcattgtattttcctcaataagcacaaagaagacagaaaagatgttttcacttggcaaaacttttttttttgagatggaatctcactctgttgcccaggctggagtgtagtggcacaatctcgttggctcactgcaacctccgcctcccgggttcaagcaattctcatgcctcagcctcccgagtagctgggattacaagcgtgcaccaccaccctggctaatttttgtatttttagtagagccagggtttcaccatgttggccgggctattctcaaactcctgacctcagatggtcctcacacctcagcctcccaaagtgctggcattacaggtgtgagccactgtgcccagcccacttgcaaaactttttaacatttataaggttttttacatacttgattttgttcagttcttagaataatctcatgatgtaggtgaccttattaactatttacaatgtgagaaaatggatagtcagtgaagataagtaacttgtccaaggtcacagcaccagaactccaaaccagaccttctgaccttcttgatacagactcctcacagtcacctgcatgccatctttggtttcagtgctatgaactttcttcactgcctgttggtctgtagctacacctactcatgaagatgcccagagccttagccgctccttgctccgccatgcttatcttatgcagctctgggaggggtgaagaggctgtggccagttacctcactaccactatagctattcaccaccctgttttctcctttagccattttagctaccgcacaattcttctgggcattttctgtcttagttccctcgtatctggccttccttcttttctcagtaactgtgacccctgctagctagccttgccaaccaatggttaatgaaaggtcttggatatgactgtcaaagacaaaagcactcatgaccccagtgtttcaggtcacaatgattaggaaaatcaggacaacaaggcttgggcacagaagagctggtttttgagagggggatttagagtttggtttaaatgtgtaatctttgaggtaatggtggaacagcctactagattcagttttctagtccctgaaaactaaaacgaggaataagttgaatcacatatttggggcaccagccaaatatatagcagcatttatagttagaatcaaagtgtttgtgtttcttaatttaaatgcttctcagagattccatttaaacaacttcattatgaaaatggagacttaccatgcctttttgatattgttcagagaaaattgtcataaataatacacacagtttgaaatggttgtacatcttatgaatcaaggggtggcatgtaaaaaggttcctcagtgcctcaatgtggaaaatatcatagaatgttattcttcaattgattccttccttcactttggtgtttatatggtatcttagatattttatgttattttaattatattttgtgcctttttatttggttaaaaatgcctactacttaaatattaacagatttttaaatatccctttgaactcttccccattcctgtacctcctttccctttttccgttgtaatgtcagccacctgaacataacccagaaaactaggtatcagccaattgatgtttcagtggaaataagagaaagaagcgcttactatttctcaatgcttaattgaggaaaagcacatgatatcttacatattttaaattggaggggataatagggtcaagttcatgcattatattgtggacatcttaatcctatcaaatggcttagtcaaaatgagaaagttaatgaatttaagcagacaagtacgaacagtgctctaaaaaaggtgctgttacaccatcccaacctgtacacacatgaaaaaaagctggggtgcattttgcgtttgaataagggtcatgttgactggccatggcagaccctgctagaccatacacctgatactctgtcacctgccaggactcaaaaggacatgggagtatgtggttaagttctctaaggactctttcttctaaagggaagcagttttataggtggtccttgtaggtctgttgattcacaatctcgtgctttcttgattggactgtattgttttactgtaattttttggacttacaagaagtcaggtgttttcatggactcttctcttttccatacagatgtccagaaggcttcttgggggaatattgtcaacatcgagacccctgtgagaagaaccgctgccagaatggtgggacttgtgtggcccaggccatgctggggaaagccacgtgccgatgtgcctcagggtttacaggagaggactgccagtactcaacatctcatccatgctttgtgtctcgaccctgcctgaatggcggcacatgccatatgctcagccgggatacctatgagtgcacctgtcaagtcgggtttacaggtaactaatgagaccaaagccagtggtttcctaccttcagcagatacctttatttagcatcttttagatcatggtgtctggctcttaaatgtcccccagctctggtgcacatttaacattatgataaggaactgggatgttccagacaactatccctaacttccttttaagagtttcagggggcagagaaagagaaagaaaaagtaccaaatactttgactgcttaaagtatatatgtcagggccaggtgcggtggtgcacgcttgcaatcccagcactttggcaggccaaggcaggaggatcacttgaggctagaggtttgagaccagcctgggaaacacagcaagaccccatctctacaaaaaaacaagaataaaaataaaacaaaattagtcatgtgtggtggtgtgcacctgtagtcctaactacttgggaggctgaagtggaagaattgcttgagcccaggagtttgaggctgcagtgagctatgatcgcaccactgcattctagcctgggtgacagagtgagaccctgtctcaaaaaaaaaatatgtacaccaggatggggaatcagagtttacttcactaaaagaaataagtacactgtcaccagaggaaaagttgctgaagttattgactatttgcttttagaaatctccctccctagacattcagggcactggcttttctggttttctgaaccctgttccttttgcttcttcattaccttgttcttatccattaaatgtttgtgctccctggagcactgtttttcgccctcttttgagccacatcacagctctccctagggaatttcactgtctgtattcgcctccactgccactgtcttcattagcttgctgatgaatctcaccatcattcccttagctccacccaaccctgacattcaggctcatgtttctagctatcctttgtatgttctccttggagatattctacaggtactttcagctcaccttgttgacagaaatacgtagcaaccatgtacatcccaaatacccacgctagaaactccctgtcccttgttctgacctcatttcaactcagtcacccaagccaacctctgagttgcctttcacctgtctattcctcccatttcctctgctaccctgtagttgagggccatgttatctctcacctggacttctgaaatagtttctgaatacgttttcttgcctttattctctccccatctcattcacccatgatattactacatctttgattataaatgcaaatattctaacaatctcacctgcttacaatgtctaatattttttcatcatccgcagaataaactgcaaactcttttacatgacttccatagctctctacagcctagactttacatctttttgtagcctggcctccccacaagcatctaggcctagtcacaccaaattctccttatttcctgaaaatgttgtacttattattgcttccatacagttacacacccttttgcctggaatgcccttttctacaactggtgattgtccaatgttatttaaaactgtgtcttagtgaccctttcatgattcctttaggcaaatggtctctaagttttattagttttatgtatcacattttattgtaatttttttttacacatatctcacctgaatagattgtgggtttttctaggtgggtctgagctttattcaaaagtgtttattaaattagatgagaaaaggaggaacattcttcattttttctcctgctttaagcactaaaccaagagttctataaatgcaataagcaaaaaagtgaaaaatgtactcagaaaactatactggatcagttagtgtagaatactgttatattaatttttcattgtattagggttctctagagggatggaactaatggaatatatatgtatgtatatatattccaacccaaagtgtcttggtggcaatcttaatatatatatattggcaatctttatatatatataaaggagagtttgttaagtattaattcacatgatcacaaggtcccacaataggctgtctgcaggctgaggagcaaggagagccagtctgagttccaaaaccaaagaacttggggttcgatatttgagggcaggaaacatccagcacgggaaaaagatgtaggctgggaggctaggccagtctcgctttttcatgtttttctgcctgctttatatttactgacagatgatcaaatggtgtccatccagattaagggtgggtctgccttccccagcccactgactcaaatgttaatctcctttggcaacaccttcacagacacacccaagatcaatactttgtatccttcaattcaatcaagttgacacactcagttttaaccaccacaaatctaccccttgtcaacttgaacccatacacatctcctgagatcacacataatcttcaaataaagacaataattaggtcataattacacctgatgtagtacaactattcttcatacatccggaaacacaccagtccccagctgaaacactcttacataaagttaacgatacttaaatgctgatgtgaagtcaataaatcttatgtcacatgataaaggagaaaggaaataaaatgaagatattttcttagtacaagtgtgtgcaagcacagacatgtttttaataaaagaaggaggaaatactgaggacaattacagtcctcatttctgcagctggtcacatggtagtagctggtattgatgactaccttcttctacccattttgtattccttttgccttcagcaaacacctcagcaggttatgtttttttttttcctgatggagaggcccaaaccttcattcatccaggtgggaccatttgtagttccgcctggattgggctgttgtagttgcccattgaccttaatcacagggcatggtaatactaagagacgccctaatggatctcctgtattccatgaatactctttcttacctccgttctggagtagtagactgatttcatcttgatagcctgggtcttgatagccttgatgtcccagccaacactgtaacgcctttcttaccctgtttacttaaaggtaggaacccaaagtgtcctggtggcaatcttaactaccagtttaatggaattgttgttgtgtcttctggtggcagcattcctccctctggaactaagacctctaggccagcagaacttaatgttgcgggaacaggaagcaaacattttgctagtggatcactaggggtgatggtgagtggtaccacttccatttccaccccttgattcctggacctgtgaatcctggctatgggagaagcagtaccacatattggacgctgattcagagcatacacagccttcttgagaactttgccccaaccctgcaaagtattctcacctagttggcattgtaattgtgactgcaaaaggccattccaccgttctgcttcaggatgttggggaacatggtaagaccagtgaattccatgagcatgagcccactgctgcacttctttagccataaagtgaatgccttggttagaggcaatgctgtgtggaataccatgacagtggataaggcattccatgagtccgcggatggtagtcttggcagaaccattgcatgcaggataggcaaacccatatccagagtaagtgtctattccagtaaggacaaacatctgcccttttcacaatggaagaagtccaacataatcaacctgccaccaagtagctggctgatcaccctgaggaatagtgccatatcaaaggtgcagtgttggtttctgctgctggcaaattgggcactctgtagtggcagtagccaggtcagctttccaccggcccatcttgttttctgcactgggaagttggggcatgagcacaaatgttaggacctgaaaggtggtgaactctgcctgggcagggcaaagccagaggaaactctggtggaggtccgtagcagtcctgatgtgcaaatcggtcatccaacctgggtgtaggggcgaaagactaattgaaccatctagtagctggttccctccaaagtttccctcgggatagttggcactctggcaaaaaccccactcttggtaccaatttactgtattagtccattttcacgctgctgataaagatatccccgagaccggaaagaaaagaggtttaattggacttacagttccatatggctggggaggcctccgaattatggtgggaggcaaaaggcacttctttcgtggtggtggcaagagaaaattaggaagaagcaaaagcagaaaccctgagaaacccatcagatctcatgagacttattcactatcatgagaatagcacaggaaagactggcccccatgatacagttaccccactgggtccctcccacaacacatgggaattctgggagatacaattcaagttgagatttgggtggggacactaccaaaccatatcattcatcaaaaggtgttggtggggtagagggtagttaggatgatccattatctccatcatgacgatgatggtattgatgatgtaagtcactgaaaatatttggtgttataagaataatttcttcctgattgtcagttttgagttgttttgctattaggaggcaaagtaggggggcatatacccacttaaaatattttaattctggctagtggaaatgacagtaatgccttcttcataattaaaatgtcactctgaaatggtcccaaattaaaacttcttcctttgttgttaagaaggatctcttcttggtgtgttctgcaagattctgatcaccttttttttttttttttttttgagatggagtattgctttgtcgtccaggctggagtgcagggcacaatctcggttcactgcaagctccacctcccgggttcacgccattcttctgcctcagcctcctgagtagctgggactacaggcacctaccgccatgcccggctaatttttgtatttttagtagagatgggctttcaccttgttagccacaatggtctggatcgcctgacctcatgatccacctgcctcggcctcccaaagtgctgggattacaggtgtgagccaccacacccggccgattctgatcatcttttatacatatgctatttttgtctatcactttaggaatcatcacagatcaaagtcatccttttggtttttgtgatagcactatacctcagtcagcttactagctcatctccactcagagatgaagaagcagaggcagcaagttagtgcctatacataatatatatggaaaccaaattcagggttgattctttctttctttctcccttccttccttcctttctcccttccttccttccttccttccttccttccttccttcctttctttctttctttctttctttctttctttctttctttctttctttctttctttctttctctctctttctctctttctctctctttccctctctctttctgtctgtctttcttctcactctgttgcttagtacagtggcgcagtctcggctcactgcaacctccacctcttgggttcgagtgattcttgtgcctcagcctcccaggtagctgggattacaggcatgcgccataaagcccggctaatttttgtatttttagaaaagatggcatttcaccatgttggccaggctggtctcaaactcctaaccacaagtgatctgcccacctcagcctcccaaagtgctgggattacaggcatcagccaccacttccggcccagggatctttctgtttcagttgtgggcatcactctgaaaatcacacttgctagaagtgagcatttatatctcttctccactgtaaataagtgcctcttagtgacatgagtggaaagacaagaagaattgcagttccttcattttctgtcttagctccctgagatgtatatgctgtgcctaaatttgtgttatagttttctcctttgatttgacattccttgataggcagagagcacttttctgtgctcatatgtcacatctcgcatctttttcccttatagaaaaactcttgtgtctcccatttacctttctatgaggtcagagatttagatactttcctagacaatcaactggagtattaacaaattcaaggagttctcgccatcccgttactggatatatacccaaagaattataaatcgtgctgctataaagacacatgcacacgtatgtttattgcggcactattcacaatagcaaagacttggaaccaacccaaatgtccatcaatgatagactggattaagaaaatgtggcacatatacaccatggaatactattcagccataaaaaaggatgagttcatgtcctttgtagggacatggatgaagctggaaaccatcattctcagcaaactattgcaaggacaaaaaaccaaacaccgcatgttctcactcataggtgggaattgaacaatgagaacacttggacacaggaaggggaacatcacaaaccggggcctgtcgtggggtggtgggacgggggagggatagcattaggagatgtacctaatgtaaatgacgagttaatgggtgcagcacaccaacatggcacatatatacatatgtaacaaacctgcatgttgtgcacgtgtaccctagaacttaaagtataataataataataataataaaaattcaaggagttctcatctctgtagtttaaataataagtgacttagactaatgacaacaaaaagccagccatgtgaataccaaatttactagttctgtgaggatatttttttctctttctctttctgcctcaaagaatctgctttgcttcccctgccatcatgatttagttttcaacccgtcagagtcttcctgctagtgctggtactttcctacttgagaaagtccacggaataccttcgagacctctgtcctcctgatggcttctatttcatttgttatatagggacccagagttccttcatcattttcaaacacatcaacagatatttatagcaaggccacaattaataaaatgtttcccagaatatatgtgtgtgttacatttagaggaaacagaagtagtattgacttgtttctatcaccagaggtctatttagtaactatattttgtggaaaatatcgatatattttatccattcaacagacatgatttgagagcataccatggagacccaaccctgccagtgtggcaggtggtataatagaagaaaatagcaaacttggtgtatctgtgtttgcgcacatgtatgtatgtgaggggcactaaggatgactttacagaggttggaacttttgagtacagttgccaagatagggagagttcactaggaaaacagaagggaagttgatttttttttttttttttttgaaatagagtcttgctgtgtcgcccaggctggagtgcagtggtacaatctcggctcactgcaacctccgcctcctgggttcaagtgattcttctgcctcagccttccaagtagctgggatcacaggtgcgcgccaccatgcccagctaatttttgtatttttagtagagacagggtttcaccatattggccgggctggtcttgaactccttacctcatgatctgcctgcctcggcctcccaaagtgctggaattacaggtgtgagccactgctcctggcccggaagttgatattcaaacaggagcagcatatgcaaagacagtgagctctgagagagtagatggatccagactcctattgctgatagcgtcctgcaggattgggcttcaatgtgactaacctacaattgcctccaggtgctccacccactgagtccttgtgtctctgctgaggtccttggagagttactggagagggctctgtgtcagattaccttgaggaggctctgatttagccttttgtaaaatgcaaagagttgaggtcttctccacgcaagagctcgctgatgtcaatgaggtattgaggatggggccatctcctatttctgtggccagtactgagttttgttatccttcctttaggtaaggagtgccaatggacggatgcctgcctgtctcatccctgtgcaaatggaagtacctgtaccactgtggccaaccagttctcctgcaaatgcctcacaggcttcacagggcagaaatgtgagactgatgtcaatgagtgtgacattccaggacactgccagcatggtggcacctgcctcaacctgcctggttcctaccagtgccagtgccctcagggcttcacaggccagtactgtgacagcctgtatgtgccctgtgcaccctcaccttgtgtcaatggaggcacctgtcggcagactggtgacttcacttttgagtgcaactgccttccaggtaaggagctccctagtgtcccaggattaggggacaaacccctagcacaggaggtagtgggtgtggctcaattgctgtttttaggaagcccaaggaaaaagggaagtgagaattttgtgtggggtgggttgctagtgagggaggagttttatgggcccactgtggtccataaactgagcaggggataatttagcatgtcagggtttatgatgatgagtggctagaaaattgtttattgtcccttttgtagaaacagtgagaaataagaggaacagagctctgggaaagggacaggcaagtctggaatggaaaagaacacgatgagaattagacactggaaaatatgtatgtgtggttaataaagtgctttaaactgaattgacattaacagtaggtgatcaactttcctatgtgcttgtgcttttgcttttgatggagtaattcattgttttcttatccacctaaatgcacccagctgcccttgattttctctgggctactggccttcacaaccctctcccatgtaccctctctgactttggggtaaccctcccctaacttaaagctagagaattctgaaactgaggaggggatcctctgttaatcagtgagcactttttgatgagctgatagatgatatatgagagactatgcgtggcacaatactttgttacactcttcactgatacaagtgttctagagtgcacacacaacccaaagatagaaacaaaaagaggagcagtgtcggggagcttggggcctggtgttccatggagagggagaaaggaacaagcctggccaattcattcaactccttataaaaatgatgaggaggctgaaaaccaagaattttgattgggaacagaatacaagcagctggagcagatgaattactaagcaacaaagatcctgtttttatacaaatatccttagtacaaaaacaaaagaaggaaaactgtaggggggagtaatgtgctaagtaagcagaattgcctcaaaaagaagttgttctagttactctttcagagtgggaatcttagattctggtattgtggatatggttcacatataatgggattgtgtgttttattttggagagattaaaggtcataggttggtcctcagtataaaatcaactggtaatttattcatttcatttggtaaaaatgtattgactgcctgctatgttctaggcaccatgctatgtatttggaatacagctatacaaagcattgtcacataattgaaatgaaaactttatattatttaagtcacaagaacaagctatttaattatattacttttagtttctcttttaataaagaatagataatgctatcattctagatactaaataagtgttttcttaacataatattactattcactttatcttgtagaagaaataactaaaatacgtctgtcttcactcctgcatttgtttgcattttaagggttaaagacagaaatagaaatgtaaacaactttattttgaaaatatttcaatattgcaaatatctctgggtctgatattctagtaatctaattggctagtaattgatgttagtgtgatttattgttgaaggctaaatgtgtttttcagtttcaagaaaattgcttttaataattgcctagaacaagaggttgatttggcagcaagatgttgacgggaagttagagaagtcaataaaggaagtttttagctgagagagagtgattattcactcccatagcctctgcattgttatccattagccacgataagaaccttagggaattctgagagtgtgtccaggaaaggatctgtcaaactagaatagtatctcctccttgagaaaggaaataaccaaggattccgcagctgagaggctgccagggctagtgaaatagagtaaggaaatcttggctgtctcttattctctggttgtagtttaacgcaagacacttatttactcactgatggtgtgtgtgtatgtgggaggggagattagcatgaggggtgggaatggggagatttgatgaagagaaaactaacatttttttggtgactcaggaactgtgccaggtacttccatgcttattagctcaattacacaaaaatcttgggaccaggtattatttttcaagtcttcccacatgaagtaactgaagtttggagatgttaagtgattcacccaaagttgtacagctaatatgtggttaggctgggtcctgaaaccgaggcagtttattttcaaagcctttgctttgtgcatcttactgcgccacattgcactgcacatctgcttcctgaaagcactttgtaggtgtgtaaaacttttcgttaaatgctttaagctgtttgggttaaaaatatatgttcatgttataagaaaaccaagacactcctaattataatcaaatagtacttgttacatatcaatatgtgtgtgtgtgtgtgtgtgtgtgtgtgtgtgtgtgtgtgtgtatatatatatggcattgtgcagatgtttaaaagtagttacatagactagttcttgcttttcagggtcccataatctaaaccagatgacttcagctttggataaatatatagaaggaaatttaaagagaattcaaaacaatagatgatgcagtgacactgtgaataaatgttatttatacagtttgtaagatttcatgctcattgttcgtatgtcccaggtggagttcagaaaatattcacttcatttccacaaagggaaatagtgcctagagatggttttcttttaaaaagtccttttcataatgcagtgcccttccttccattgcccttcattccattgcttcccatgcttgtcaagagactaaaatgttacttatagtaatagtcactatctcaatgtaaatagcacccttatttgatgagaattattatttcagttctaaaaatggggaaacaaagtcagcaggaggcaaagtagcttgttaaagtattctgcaactttcaaatggttgcttccactgcatttcacgtcttggcacttctaattgagggttactctaaccaccctatttaaaattgtaactgtcccccacccccttaattactaaccctggtctactttttgttttctttttctgtaactcttatcttcttactatataatttatactatataatttacttcattatgtctattgtttattgtctgtcttttccaaattctactgcctcttaccctctccagaatgaaaactagtatctttgtttttgtttactgatgtaacccaaacacctacaaacagtgcccagtatatactaggcccgcaaatatatattggctgactgactgtatggtttagtatcatgtcatagtattgagactgtaactttggtcttctcattttcttctttgtattgtgcgtcctagacttagtttggcctctccttttgtccttgtatactctaatactggataagaattttggagtctttttcaactctgagtcagtgaatgccacataacttagtgactatatttaaatggttaatttacaattttttccctgcaaaggatactgtagtcactgtgagtattttagtattattgtaggactcaagagggaattaaaactacaaaaatgactcgtcttgtatgacacagaaagaaatgtttcttcacagagggaggagaaaaatatcttcaagagagaactaattgaatcaaatcaatgaaccatgtctcatctttttggataagtaactgttagtaatccagacacttcatgagctttcattatgtaaagtctttagcagaagctaaaggaggggcaccaaccacagtaattttaacttaagaacaaaatggagcatgaaaataaattattaaatcatttactcccactatttttgggttagggccaataatggggagagaaagaggtagactagttttgtgtttgtggctattttaatagagtagcacaagtaatcaaaaaacagtaggctgttttgaatttactggctgtcccttatgagttcacagttagattggactgtcctcaatgtactttcttttttttctttctttcccacatctctttatttctctgattttgtttaaacttcataaagagctctctgatctttcctttccaaacaatgaaggtttatcctttgtaaactacctctgtactccacaggctgatgatatatgatatccctatatcattaaagtaaagcctaagcacattctgtggcttttgtgtctactctgttgttgctgagcttatgaactattagaaataattccctcttgcattttcacacatggggaatgtgatgttctcttgggtattatgctaatcatattttggcaggtttctctgaagcagatgcagaaatgatcataccactttccagggtgtattattttagctcctttgacttgggccctaagtctgttttacctgatgttcctgaaagatgttcctgatgtccctcactgttctttcatgctggatgttcttgcctatgctgcctcctcagctatcaccctctcttcccctttttaatgtagaactcattcttaatgatttgtcaaaggcaccctatttcactgaaatgccttctatattccctaccctccaagtggattgtagaccttctaaggtcttttgacatctgcatatctctagcacagcacttatcacggtgattatttatctgttcatctttccaagtagacactctcattttaactccctaccctagtcgccagcatccccagcatagtgcctgtcataaaatggtgccacaatgaaaatttgaaaaatgaatgaattgaacgtgataaacatagatgagaatcctatattctacaattttttaaatgtactgaaattattctttttgaatcctcctatttatttctgtgacttctttggtgacaaagttagaaaaaagtggaggtcagtagggagatatgaagggacgcaggtggaagcagtgagcctgggcgggtgatggagtgggcgatacgtggcacaggggtcagtgagttaatctgggctcattcagagaatggaagttgtgtgccaagaaaactggttggatagggataggtcagggattccctcttgcattctcacacttgggggcatgcgtcattttcttttcttttcttttctttttttttttttttttgagacggagcatcgctctttctcccaggctggagtgcaatggcgctatctcggctcactgcaacctccacctcccgggttcaagctattctcatgtctcagccttccaagtagctgggactacaggtgcctgccaccatgctcagctaatttttgtatttttagtagagatggggtttcaccatgttggtcaggttggcctcgaactcctgatctcaggtgatccacctgcctcggcttctcaaagtgctgggattccaggcatgagccaccatgcctggccgcatgtgtcattttcttgggtgttatactgatcgtatatttgcaggtttgcttttgtgacagacttcttctgggggaaaaaaagtatccttctatctttttacttttgtccagttccaggtatccctgtttttttcttcactcttccttccttgttcatgggagtttttcttgaggacttcaagcccagcttcggagaatcctggttgtgtcatctcatctcctttctgctctcttctctacctaggctttccaccctcacacctcccggggtctgaaaatggaaagataagggtgtttccctgaaagttgctcttctgtgtggggatgacaggttctaaagactcttttctggtccctgccctcattgccatgattaatcagttaagtggcccgaggttttgtaacagcacagtcttaaaatgcttctcccaagtttaatttctctccatttgaccttttaaggatgtgaattggctttaagcagtagactccctttagtacggcactgtgagcctctcagtgaatctgctacatccattccacccacgggtctggaaacttgtctgtttacctttccctaaaaacctaagatatatttttaagaagtgccttgtaacttttcatatagcctttcccctactttgggtagactgtttcttacaggaatttggtagatctttccaaagagaattctgtatctctatttttaaagcataaatcctgtcaactttggaggagaactgatttggcttgagtcttctcagacatgggaacttttgacctaagtttgtattttacattgttgaaagggaactccgggatcccagaaaacatatggactgcaattgggtaaagtttctgtttcagtacttattcctacttactagccgtttaatcttggtcaagtcagccatgtggctctcaacttcctcatctgtaacataaaaggattagagtagacaatctctaacaagtgctataataccactgacaaataataatattagctaatatgtgtaaggcactgtgtttagtgctttttcccttaatacaatagctttgagatataatttatataccatacaatttacttcttaaaaaagtacacaattcagtaattttagtagataggagtaaccatcaccacagtcaattctagaatatttttatacatcagaagaaaccctttacccattatcaattactctccattcctcctaactccctcccagccctaggcaactactagtctactttctgtctttatttgcctcttctggacatttcatacaaatggaaacatgcagcatgtagtaatttatgacagcttctttcacttagcatgaggttttcaaagttcattgatgtggtagcatttatcagtactctgtgcctttttatggctgaataatattttatcatatggatttaccacattttatcattttatttatccatcatcagttgattgacatttgagttgcttctactttttgagtattatcaataattctgttatgaacattcttgtataattttttggtagacatttatcttcatatttcttggatatatacctaggagcagaattgctgcgtcagatggtaatgctgtttaaccttttcaggaactgtcagactgttctgaagtgggtacattattttacattccaaccagcagtgtatgagaattccagtttctccacatcctcatcaacagttgttattgtctgtcttttttattatattcgtctgtaatgtgaagtgtttatctcattgtggttttgatttacatttccctgatggttgatgattttcaacatcttttcatatacttattagtcattatgtatcttctttggagaatgtctgttcagatcctttacctactttataattggtttatctttttaatattgaactgtaatagtttttaaaaaatatatcctaaatacaagtctcttatcagataatatgatttgcagatattttctgtcattctatatactgtcttttcacattcttgatgatatacttttcagcccaaatgtttttaacttgatggaatacaatttattttttcttttgttgcttgtgctttcagtcatatttgtgaaaactttgcttatcccacattacaaagatttactatttctaagtgatttataattttaccacctacctttaggtctccgatccattttgagttaatttttatgtgcgaggagggagtctaacttgattcttttacatgtggatatttagttgtcccaggaccatttgttgaattaagtgctgtttttatctaattcgtttaatctttacaataaccatttaaggtgggtcctgttaattccccaagaaagtttaagtatattgcccatattcactcagctattgcatggcaatgctgagagttgagcctagacagtttggccccagaagccatgcttttagctatgaaataactgtcttacctacctcccttcttacctacctatctacctattgacagggaaaaagtacctcaacaatagtcaatcagttataaaaagaaaacactttatttcttttctgttgtggttctgctgaaatgcttctgctttcttgtctgtggttggtgaattcagttagttgtaacattggctaatataaagttagagtcacagtttctattccttatagttgagctacaagggcttcgctacctccctgacaccctagtcaactgtatttagtcttactcatcagagagatcgagttaggggaggatgatcattgaccttctactacaccaccagacctctcaagtttagcagttgtaggccaggggccccattttccccaacaatctaaaactatgtctttaaattttccaaagaatatcttcatcaagagcaccagactaggggcaaagaccctagttcttactcaaggacctagtagatcccatagattctgtaggggtgggcttcaatggctcatcattttctggctcattggtctatttgtaagactcatatttctgtatgaataatatgaaatatgagtaatatgatacatttttgaaatttgagtaaattttcatagtctttttcaggcttcttcagtgattcatctgcttgaatggactgagtaccaaatacttggagaaatataatttccttcaataaagccttcagtgatttctgtcttccatggagtgatttttggctaatgtttttggtcactcagtttatacttatttatatgactgccattgtatttagagaaatcagccatgttccagtgtagcagagcagatgtagaatgccacagtaacttatggtaaggaataaagcaaagtgtgattgatgtgtgtcccactcacaaggctgtaaatgccacaaaagcagggactcactgctgtatcaccagtgcctaatgtaagtcccagtaagtattttgagtgagagagaggaaccttaagggatatttcgaggaagccaaatatgtaatcatactatatttcaaataggagaatgtttagaatagaagctggtagtcttctatttaattagctcaggagtttggtagcaaggaaagagaaagtaagaggcccaacatataaactacaaaggaccacactgtgggcagttagagtgaagtttggtgtactagtttcatttttcataaaagaaaaagtcaagaggaaggaagcaatgctgtaagtttagagaagagcctgatgaatttgaagtgctggtaagcttagttagtaataatgccacccaattttcagataccttttaacatttgtatagcactttactcatgatgtggcacttataattagaaagacaggataggcaagtggaattagcctcattttttacaggtgaggaagttgatgcagaactttagcttaccatgtacgaatagcttgcaggtggtgagattgggacttgatccatatcttcatactttaagttcaatatttttcctatatcctcccccactcccttccccacctggcttggaggccgcctagcaatcattcaaagaaaaaaaataaaaggaacttaaatttaattaggtaatattttatctgttcttcccattagtctggtatggagctggggaaaatccaataccctgtttttttagaacccttgaatatctctgtcccaagtagacatttatcatccctgggattttatttcatgaaaacattttgaaatacttgtgaggtaactatgaaggcatgccattggcacaatgagtcactttcactgtctagctatgcttgaggccagagaagacagagaattggggatttttcatatggtgagtcctgggtgagcagctgttgaactgaagtcatgaagcgggtctattctaataaccatgttctgtgggaacagagcaggtcgtttcttctgtatgagatttcatcattttcatttgttcttcatttaccaggttttgaagggagcacctgtgagaggaatattgatgactgccctaaccacaggtgtcagaatggaggggtttgtgtggatggggtcaacacttacaactgccgctgtcccccacaatggacaggtatgtacagtgtggagaatccaccagaatgggatatggattggtaaccagaggcagactgtttttttaaaatatcagtaacaaatatcttaggcctgctctggaaccagtatgtgaactgagcaggatctccatgctagatcattgcttattgtactaacctcatacattttcctatgttcactcaaagacatattttcacaggtattattgggcagcaactctgaacccagcaccatgctagatgctgtggcagatatgagagaagagcactactgtaagcttattatctgggaaaagcaggacattacctggaacatagtagatgcttaataaatattaaagaggaaaaatttcctgaaatgctaaaataaattgtacaggctaccagagttcagagcctatttgttcacggacagagatctgtcccatcttaatgtcatgttctataactaggcaaagtgttaggcaggtactcttaatgacattatttttatgatacgcatcctacatttgtttttttcttctatccctttttgtacacgaacaaaaatagttttttctgcctctaacattagaagggtagtcttgcttaattttacagcctcttggaagtttctcatgtagtcttaggcaggttgagtgatttacgcagacatttggttctaatttctctttatgtctgtttatctactgaaagtagagacaattaagaactgattaagggactaagagctgggcctttttgagagaaattaatttaatgtggaaacccaacaggaagttagtattataagaaaaaattacagtaccagtagtgggctatctcctacaaagaatcttaatgttgaagtgcagccattactacctggtattctttagtgtctctgaattacttttccttatctgtagcatctaattatttacaatactctttagagctgtagactatgaatagaaaaagatgaaaagcatttaaaaactcaatcacaggacttactggggaggccaaacatttctgcttttaattcccaaactgtcccttttaatactttcccaccaggttttcctagaattgggacttctcctagaatttcttcatggtgtcttgatcctagaacttcacttctaaccctctgtactttttgtgacattgtctcttctagtttttgccatttttctatgatagacttttcagaggctattttttcaacttaaaaaattactgtaaagttaattacatgacatatagtaactggaaaaatttgtgttttttttctctttttaatgatccttacagtattggacaggatgagatacttgttaagaacttgaccacattctgatctctcccttgctctgtgagacagtaactgtgtagtactgaggcttctgatgccagtgaagattaaacaacattgtaatattgttgctggtttttgttcgatactacacttggtttcttgctttgatattaattaggaaggcctgctcttggggaaaacttccccagtgaaatgatttttttattcaaatttcttatcccaggacatttaataaatgcctcttttcgctagaagtctttataagatgtttccgtttttatattttccaaaaagggtggtggagatcatggataagtttaatgtggcagttttatttcttttgatttgctgtctgtcatttagagtgaccttcagttttttttttgttattcatcacaggttttcttcactcatgccaattctttgattcccagtgagaagccttgtttttgccctgcccatcatcaacaggagaaagtctaagatccttcttctgttatatgaagcctttcatggcttcagctctgtctgcttccccagtctccttctcatgcatctagcagcataccctagcagttctctgctcacagttttcctgtacatactgcatagtgcttcacatcctgtgtcttcattgacaccattccttttctctagggtttccttctccactcctcccccatttttctcatcatttttggcatgcctgactcagatcaagtggttacctgcttcaggaaggcttccctgatcttgctccaggctgagctaaatgttctactctggactcccttagtaccccatgtattctcctgtaataacacttaccacgtttgtattgatattattatatgcttgatgtgagggatgtaccgagttatgggggcggttggactattgatgacagcagttactagcactccttacagacctttgttctcagtagcatgtagctgagctatttaccatcccagcccctatcctattgtaaagagaaaaacagaaggaaaattttgtgtcatagggcttctgtttctactgatattagatgtataaattcagattagtcttttctgtgttacacaaattaaagaagtgaatggagcaggaatctagtaggagttaactaatgtttattagcatccaaatactgtgctaattacaatgttttatctgtgtaatctttgcaataactgtattagagatagagtttaaatatccttttttcagatgatgatgcgtattcaaaagggttaaataatttgcccaagacccgtagtggttgggttagggtttgaatctaggcttgttgaactccaaatctttgctcaaatctgtagtcttttttctatataaaattgattttgtaaattatcttgttttattttaagaacttggggtgttttttaatagactgaaaatttcattatcctgagatttggtgatttactccatgtgttttccctttttaagaacctagctggttattttgaggttagaagttaacattatgtgtatgctttagattcagtctgatgctttttcactatacctaatagcctattgcaactttgttcagaattccttttacaatttccacctgtagacttgaagcattaactatttggtctggcttttgacaaaatgaaaaaggaggggttggagtagtgaggtgagaaaatgaaggaaatgaagttgccagctatttgaaaaagaatggataagactatatggctgaaaaaagataggcattaattttaaataaatggagatttggaaatttgccatgcttttaatgagaagaacctgatagttacggctttgttttgtaactacatatacataaaaggcttggtgttttgtagcaagttctacacttcaagatgagcataaattccagctctattcatggtttgatttagggtaaatttgtcagcctccttttctcagaagccttcatttgctattatctctttcttgggatattgaaacaatcaaaaatgcaataaaaagtttattttttcatttgttctgccaacagctattatgctaaacattctaaattcatcctattctatatggtggcccctggtttcatcttgctattgagcatctgaaatatggctaatgtgaattgagatgtgttgtaattgtcaaataactgattttgaagacttagtatggccgggcacagtggctcatgcctctaatcccagcactttgggaaactgagatgaatggatctcttgggcccaggagtttgagaacagcctgcgtaacatggcaaaaccctgtctctacaaaaaatacaaaattagctgggtgtggtggcgtgcacctgtagtcctggtcacttgggaggctaatcacgccactgcacattccaccctaggggacagagtgagaccaagaacctgtctcaaaaaaaaaaaaagaacttcatatggaaaaaaagatatctttttaataatgtttaaatattgattagatgctgaaataatacattttggtacattgaattaaatataacataatattaaaattaatttcccctattcctttttaccattaaatattttagtagccactggaaaatttgaaattatatctgttactctcattatatttcagttggacagcattacttcagatgcaaagatggtctgtaggtattaccattgctgctgctttgtagaagcatctgttctagccctgaagtagaggaatagatggtttccccatggcctgtgggcagaactgtgttatataccccactacaggcgtttttacctactgtggctgtctaattagtgtttgtttgtgccttcaaaaactaccactcttgtggactagcttaagctcagatggaaaaaagaaattggtcttagggagtgggtgtggggagatgatttacttctttatagggaagtgtcttatagggataagcctgaatactggatattcctttaggaaatagaagtagacctgatggttggatgaagatagagcacaaacgtctttattatactgtatttggcctcaggccatcttatcagagaagttctgctctggcagatatccatttaaggaaaaaagggttttaaagggcccaaaatttattagagcagtgtagattaatgttttgtctacatagactgttatttgaattttctttttaaaggttcatttccttcagaattaaagttgaccctaagctcaactcacaaattcctctctgtggctgcttttgactttgggctcacttctggggcagatgtgatttattttcttgtgcttgttattaatccagaaatataatttcagcatgggcttggaatagtgctctgggacatgaagcagaagtgaacaatggaagttaggtggatatgagtcaaagaaaactgaccacactttctctcagctctcctttacagcatgatgagcctcaagagctgggggaggttttcattgctgtcagtgctcccaattcagtaaaaatgtttgatgtgagttggcaagtacatttgccagaggtggtgcatgagctccttgtgtctgggaatggagattttcatgatggtgcttttattttgagctagatcttcctgtctcagtctagcctctagctggtgatgaattattctctagagtaaggcttttctcacaaccatttttctacttttttttttttaggactatagggatggaggttttttgattgagagagaatactacttaattatatatggtatttcaaagctgctctcttttatattctacatggaggaatgggaaaatatgtctcacatttcaatttttctgctgtaagcctgattgtgagaagtataataagcctgttttctgaattctacctcctcaccccaaattcggtttcctatcagttgggcagactgccacagtgctaacctgaagcacccttgccccagatgtcccagagttgattagtggcgctgttggtggaaacaaggacgagtggatagtctagccccacatgcaggaggtgagactgcaggcaggatctttcctgccctgcagtccatgactcagggtgtagccttggctatgttacaggcacgatccagtttaacttcatggctctctaagcccccatggaacggttgttagtaggttcaataatttatctattggacctacttacagatatttattgaacaactactacatggaatatattatgctagatactgtaagagacacaaagaaaaaattcctattttaagagtggtaaaattgactaggaagacataagaaagtccacacataaccacactaggaagcagaaggtgtgtgtgaatgtgaatttgaaagaaacaatggtgggaatggggaagcatctaagtacttggtggggtaaggaaggtgatcgagaagagtgaacttctgttttgagtctgagtaatggatgggaactgttgacagagaaggagaaatagggtagaaatgatggtaagttggagatgagttttccaagtgaaaaacatgcagcagtaagacccaggagctcagaatacaggcagggacgagagactcagggttaccatggaatgtgtcaagtctgtgagggaaattggatggagtgataagaacagaggatttggacccgaagtatgagaaacatccacacttcagaaaattgacgaggaaggggatccaaagaaggaaagtgagaaaaggggaggtttgagaggtgggaagagagctcggccatgatagcatcctcaagttaaagggtacagccttagaagcttgccctgaactcaactccacatgccttggtggcagctcctgtaatattttacttctcttactgcgttatttgttcagcttgcctaagagactatcagcctctggaaggcaggaaactgctcttacttattttgacattcccagaactgattttaacaaaccatggttattaataactgacaaccaggagactcaaatataaatgataactgcaaaatattcattagaatatttgagtcattagaaattttctgaaatacagttttaatacagacaagactaagaagctagattgaggaagttaaaggaaaaagtaggctttggggagaaggaaggggcaaaatgggtttagatcactatgtaagattttgcacaactgaaaaggaagagcgagggagacacaacgcagggttatgggagacagaaactttattcatctcattgaccatagagcttggctacactaagagtaaggcagctcaaagggcgttggctaatgaagtggattattgggaggggtttactcttacagtttttagtggggaatactttaggtaagatgtctgctgggaatgggaaaggaagaagatgggaaagttatagtcagagagtggaagtaccaagtttgagatcttggactagaacaatgagtgtcttgtaacagaggtgtagaatgtgagcgtgcttgtgatatggaatggatgcggaggtctgtaaggttgagaaaagtaagaactcttgactcagatgaccttactggtcctcaacccagatgttgatatcatgcgagacaggagaggaaagagcgagaacataagccacatgcttgtcattctaggaagaggaaattggtccacgagaacgacagaccatggtgtgataatttatattatactatgtcttttttttaatttttaaaggacttcaattcatctatctacttttaatgtcagaaattagaatgtatttattgatttctttctctcgaagatgaggaattagcatacttctcatagctttcactcactcactagactttgtataaataatctgggtttcaatatttatattattgttatcaaatttataaacatatacatattctttaaaattattcttgatatttacaattttataacccaatttataatattttcaacttagttgtatgtgctacttgttttgtgttaactaccagtgcaattatgtcacaacattttcatttctgagttccttattttcattaacattttagctgaataagtatctttgaataattttatttcagaggagttacatgttggacccatttggttagtcaaaaaatgtcttttgatttagttgccttcatacacaagttgtatcttgactagatatcaaattcttgggtcacgaattaatctcatcaaattcttagacattgttcctttgtgtcttctggttactattctagggaaatgcaatgccagttgagtgtttcttctttttcataagtaacactgggctccctgccttctttttcatttgttgtagagttgtcaggaaaacctattatctttgcaattaggtcttcattctttcttctgcaaagctaaaatctctcttgatttttaatctgttcattttttgttttgtgttctgtgagagctctcttaaggttcttctttataacactgacttatatttcttcattgtcaggtcagtcctttatagcttcttctgcctgcctctcagcccctttgatcagagactaatgtcctatattattttttatttctttgtagaacgtaaacatgtctaaatgtcttgagtgtacaggagcagttctttctaaaatgtattagctcctgtaagtgaataattttcccagtgtgcttttcatcaatatcttaagcattacattctccatgttttatgttgtagaatcttttaataggtgccatgttgtttttttccattttactcattctgagcagagtaactgcctgcccatagacaggggaggtgggtgttcccctggatcccctcactctcaacctggctgcagctagtttttcctcctggtgcagtgaggggcttttatgtaaactggctcaacaacccagcagcctaaggtggggagagccagagagtggggcctcctagcaggagaccaaagcttggcaccactttcttctctgtggcgggggtcagctttctgaatgcccagtactggcccagcctccatggcaatccacccagtggctgagcctggatgcttttcagtgctgctgcctccggaatctgccccagatgcactgcagtatttctcattgctatagtggccaatagtggccacaggagcctggactctgcagttcttgaagtttttgttgacctggaggggtgaggtggagttcagtccattctcctcactgcagcagagtggactttctgaaacatgcattggatcgagtcatttctttgctggaaacacttcaatgggtcctccttgtctttggatttagtcccaaggctgtgcatgtaactccctctccagccacacctctcttcactcccctaatgcatttcttataattcaaccacaagaattttttttcattgccgcaaatgaaccatgctgtctcttcaactcaagcctatgctttctgcctgaaatactccgcactccaccctttctctacacactccctcacacacttctcccctctttcacaacctggcacgctcctcccaggccttctcttaaaagttacattctctagttcctctctattctccctactctgtactcccaaggcaataggtatctatgaaagcacctgtcactctgtgtcatcattgtttgtttggttataagtctgctgtttgtaagttctgcaggcagaagccatggctgttttgttcaccattataatcccattgtgtatagtcagagcctggcacattgtgtttgtgtgcgtgtgtgtgtatgtgtacatatatatattttagataaacgaatgaatgaattacagcattaaattttccagttgttcccagcccaacctcatctccaaattcacagctcttcttacctttctaagatactttagtatttttcttcagtgtgtaccgagtttgtactaaatctgtaaaattagactaattatatatgaagactatagttgacaatcaatgtaaagcacatatcatctggaacctagaaataactcagtgcatatgtttttactgctgaggttagtagtgacgttggggaataaaaagaaaacccgctctttctcccctagtttcctgtcagtggcagtgtttagaagaggaggactgcaggagctctaccgcagagggctgactgggctggtgtgtttagggaaaactcaggtttcagtcaaagaaggaagttgtacaccttaatgtagagttgcccttgaggcgtagttgaaggattagtgggcaaaggcaaggtgaatctgggtgtgatacaaatgagatagagttcagtgacacaggaaggggactgggatgaggggtttgcgtgtgaactggtggtgtagacaagggtgacggacactttgggtaggaacagaatagagatactgttgctatgagaagggtgagttgaaagataattattattttgggattaggataacttaaataagagaaccttaagcagataaacattcatttaacgaatatttatttaagcttgggattgtgtttcatgctggggattcagtgatgaggtaaacagctaccccccatttcttcatggcactgacagtctagtgagaaacacaggcatagacaatataattctccataaaattttgaactgtactaaatgccttgcaggaaacatagaatgtatctggagagcatattaataggcagaactaattgtgtttagtgacaagcaagtaaggtctctttgagaaagtaacattttccatatagacaatgagtaggagttggggagagtgaggataagactatcccaggcacaaggaataacgtgtgctagccactgaggagggaaagaatgtgatcattttgagaaatgaaaaaagcccagagtggctggagctagtgatatttcatggttagagcctaaagagataggcagaaaacactggtaggtgttagaattttatcctaaatgcaaaataattcatcaaaggatttgaacaagcgagtggcgcattctactttacatgttaagatcatcctggcggctctttagaaaatacattggtcatgatgcagggaaaccaggcctttgagaagtcctgaaaaagatgatgctgtctagaattagtggaggaatttcttgcatttattctgtgtatgaatatcctttccacaactacattatccccttagccacatctatactcccagaccatcctctcagtaggcaggccctgtagacagcagtgtttatacagaatgtttcacccaactttattccgagcaggtgatgggacactcaagcttgttctttgttcttcagctccaacttcagcagcgtgatagaaagtctcctgtgtatggagctgaggttttgtaggatagaagtcccaggaatgatctgctctgacacagaaagagatgaaactccagaactcaggggcttgggtgactgctggttggaaaggtatataggagatggagctctcttgactcagccatagaggagaaggtagcagactcagtgcaagggccagccaagctcattttttatttgttcctcttgtggagattgttgttgcaatgagagttgacccttaactggaccagtaatgtgatcagactttttcccacagacttcacagttgagagatgactggaaaggatgagagtcgaggctcagcaccttacttttttcaattcctttctcttcttttctttctttctcttgttgtccccctctattttttacccatcacttgtagctccaaatcagattagtggtttgtgggtggggcccttgccttgagagagccataatgagctgtttgtgtccctgcgtgttacagctcataaatgctgactgtttcatggtctttggctcacttggctgaagcgtggtgctaatgaggcccaggccatggtttctatttcagcacagacctcttaggttcaccctgttatatttactgccacaggctgtgtccataatttttttttttttttttttttgagatggagtcttgctctgtcgccaggctggagtgcagtggcacgatctcagatcactgccacctccgcctcctggattcaagcgattctcctgcctcagcctcccaagtagctgggactacaggcatgcaccatcatgcccagttaatttttgtatttttagtaaagacggggtttcaccatgttggccaggatggtctcaatcccttgacctcgtgatccacctgcctcagccttccaaagtgctggaattataggcgtgagccaccacgcctggctgactgtgcccataatcttacttgttcagcctgaagccagaggatggagcaacaagaaaagatcagcatggaggtgaccctactcctgcaaaaccaaaacagtatacttcctttgctaatggtggcttggtaatgccatgtcatcttcacttgtagcactctgagtttcttcctctttccctacatccctcaacagtcagctcagattggccttctttattttgaaaactaacttgatctgcagcaatctcacaccccaaagtctttatcagcactgagctattttcacttctaattattttgccaactaactgtgcaaagttgggcaagttgctatacctctcaggtcctagtttcctcatctgtaaaatgagcatgttagattagatgctgtccaaggtcagttccaccactgacaccaagtgtctagtacagtggctggcatagatactgaggtgaatggcagacacagtccctgccctcctggagcttatagcgaaatggggaagagactgtaaagagtaaataaatatgaaatcacagctcgtgatacattccaggatggattgaatggaatgatgcaatggtggagaatcatgaggagatgtgcatttggagacctactctattgataagacaggtacctcgccatgtgaataagcagggaaaaaatgtttcaggtggacagagcagcttatgcagcagcactgaggcaggagagaacttaatgtttgaggaatcaacaaaagttattgtggttagtataaactgtacaaaggagaggtgtagatttgagtatatttacgaggcaaaagtgatcaaccagaaaattgtcagtagttacaggtggcactcagtgtttaaggtatcagccaaggatatgctagtttgacatttcatttcagattattccagagtcattgtggcattagagagggtagtttttaaaaacaagttatcttctttctcttttgcccataggaaataagagcctaactagtccactgcctcagtcctgatgttaaaaaaatgacagtagcctgcaatatctggcaggtggaaattactgagagggataattattgctcttgaaaacttgcctaactgagctttgtgcaacattttgggagttcaccagcaaagtttgtgtgttgacacaggtgatgatacatgctgacacttgtttgtattgaagatttagtagcttggtaggacatggaaacaatacagctctcctgtttcaccaaaggcttttatttgctaggtagcctataacttagtgctctctcctgctttccctactaacataaagtcatttgcttttaatcccttctatgttctattggccaacttaccttcaaacacagtatcgatcatgtctttcctcatcctcctttacttgttgtatcaagttccgaattcatactcgaagtcaaagcctcaatgacctcccactttttcttcttctaaaaccccagccaaagaatacactgcagttattttaatatgtttaagacttaatcctgtattcctctgtaaatcttcaaccaccagcccaggctggtgctttcttgcaccagtgggataagctcaatgaccgtagagcatacacgttaagacctctaggtgatcgccaagaacttttcccactcaaatatctagtgaccttctgcctttattcctttctaatacgagtgttccttaactgtcttatgcacctatgccttctcagggtgctgtaagaccctgggatcagcgacctcatctgtagattgtgttgatcacatccagggctgaatactcaactctgctgcagtcaaagagcatcagatttgcagtcagatcaatccgggtttcaatgcagcactctcactcaccaaccgtgtgaactttggaagattttatttccttttttttaccttcctgaacctgaagttttttatttttagtctttagcattggttttctcctgtgccaggagggtggtgacgtgaagggagttgttatatcatagcagatgttccactgggaattttagaataagcaagttttatggccaccctggttactgtttttcctattctgaattttagaaccagttgatagaattttaatagggagaaaataatctggcagaaataggactaggaggaaatgtgaatttgtagaaatgtgcctgaaccttagagaagtgtgatccatccagtagctaattacttataagccactatcttcttggatctctaaatttcctgtccctaacaagcactatcgaacagatgcatggtccaaaggtgttcagtgaaggcagctaatcttcaaaattcagtctcattcaaaaatcagtggactgtacctaaaaatacatttcccttttagacaggagtttgcagtcacaagtgtttccagtaaccaattgtttgagtcatgatttgagtcctcacctcctccatcctgtccacttccccatgtctgtgtgcccttgggaagcccgagacatgcagagatggcttgatatgttcaggttctcacctcaggtctttgttaagaattttagagtgaggcagctccagttcaaatctcaactgtccttcactcactggcttacaaaggcaaattattttacctctttgaggctgtttcctcagatataaatgagacaatttttatgggagcccaagcacagtgcttgcttctcagtaggcagtcaatggctactggtttcctctcccaagagtgccacatttcctttcctttatttacctagtttgtcttgctacattatttcagtgtctgtttattatggtgcataagaaatgtatacaaaatagctttaaacacattgtgtcttatagtacccaaaattatcacaaagcctcaataaagaaaaatgacatctaaattgttcacattcagcaagtcagtttggttggtgggcagtggatatttgagttagcataagtataatagatgagactcagcaattttcatgttgcagtagctcttcacctacttcttggtcatgttttcatttctcaatcccacctgcatcctaataacacagctgtcattactttgaggtaccatttctgctagcactataagacagcaaagtttctgtaataaatgacatcatctaccctgagggagattattgccggtgagctcactccctccctccttcactctctgattccttcactcactcactgactccctccctccctccctttcttaccttcctccctccctttctcacctccctccatcttttactctctcacgcattccctccctcgctcactcactccttgcaatcactccctcactcactacctcagtgttctcactttcttattcatcttctcccttccttcctccctcatttccctcattaccctcattacctcccccattcactccctcatccctcactcactctttctgtattaatactttgggatgcagttgacacagcctcgtttcaaattagaaagacctgttgcaagaggcatctctcagaatggctatgccactgagtaggcttactagtagttgggcttcagtagcaattggaaccaacaacttgaatgccaccatgtttttttctctttgtgcacatcttttctatttttctttttttgagacagggtcttgctctattgtccagactggggtgcagtggcatgatcatggcacaagtgattctcctacttcagcctcccaagtagtgcctgcaggctggtctctaacttgtgggctccagtgatcttcctgtttggcctcccaaaatgttgggtttacaggtgtgagccactgtgcctggctctctgtgcacttctgattcacccatatcactttctctgcagaccagcctcctcatctttttggtctatgtggcaaagtggtcctactccaataatttgggagagatacagtgatgtagcttggatcagatgcccatcttgggtcagtcagccatggccaggagggtttgatcatgtaaagacatggttgctccttcaagaattatatggctggagttaggagaagagcatggaagggttatgccgagcacggagttccggagacatccacatacccatcctgccatcctaggcagttggctcttgctgggaatacaaaagaacccctctaaactgcagccaagtagtgggagacagatgtgcaaccaaataagtgtgatagagtgatattggttctatagtaatggaggtacatgttagataggaataaagaacaggcagtcagttaggttaagtcagacacaactagcatccgcgagtaatgatggctaacaggccttctacagatacatgagccagtcaaattgctaggtattctgggtctagagatagccttggcattttccaagcacaaatattgtgttctttttttattaggaggcctctttatcagagtgccttttaaaattatttgttgagttttatttacaaaggaagaatgaaggacaccttgacatatttttattttcaacaacttgtcatgttttagagaattcggatgatctaaattttagttcctttgatagctctctaaactgtttttaaaagacctcaaacaaaaccaatcaccactttattttatagctcatatgatctggccctatactgtttttgttaatcacagattcctggttgtaagttgaagacattatcttgttctttgttaagggatgtaggagcctttgccttagtctccattgttgctatagtggtgacctgcaacctagccagatgtgggctcatgtttagcttcctttagtgtgatctttttcaggcagtgcataccagtcctttttccatctcctagtacattgggtggactttctttgtcttcttccacaaatccccatcctttaagtatgggtgcctcaggctgagcacagcttgctccagtctcattattcttggccttgcccttcacatccatacttcaccagagattttcaacatctttcatgtcttctctgtaccctatgttgcttgccctaacattttcaaaaattagactttttattttcagatacttgtagttttgtgcagttataagaattaatatagaaagatttcctgtaccctttatcaagggtctcccagtggtaacatcatcttatggaactgttgcataatattacaaccagtgtattgacgttgatacagtcaagatatagaacagttaccctaatgtttggcatataggttccttctcattattttttcttactcatttttatactcttctctccttgctcttcttacttatatgttcattgacccagggattagttaactcgagggtcctacaggttttaccttctctttgataagttggctaaaccctaatgttgttagagtttctaacggtagtaactattcatcttgaaacgatttgcctggaaaaaatatcatttagatggctcaaattggccttaaaattcatgtgaagacccagccttctggagctctgcttgccttaatttcagacaggtcacatgaagatgaagagtgtggggggctggtatggtactagtcctttgagactcaagagccctaatcaaaagtaatttccccctaaaaggacagttctgcacagaggatgtggatgaatgcctgctgcagcccaatgcctgtcaaaatgggggcacctgtgccaaccgcaatggaggctatggctgtgtatgtgtcaacggctggagtggagatgactgcagtgagaacattgatgattgtgccttcgcctcctgtactccaggctccacctgcatcgaccgtgtggcctccttctcttgcatgtgcccagaggggaaggcaggtaggtagcagaaggacggatctagaaacagggaaggggcagggggcattcaggactctgccttctctttcatcccaaacagcatattctttgagttaccatctatgggaacatatcactcaggactgttccagaaatgtaacctgctagccaaagtgctatgaagataatcttcctctttaggactttaggctctcttctcaactctacatactttagtttttcattccaaaaaaggaaagttagtaactattgtctttggaacatctgctgaagtccttaagatcttagactgaaaagcacctcagctattaagataaaatggctaataaatggatccacaggcttcattttaagaagaaaccatctatttactcagaagaagttggcaggagagaaacatttaagtgccttctaaccctcaatgtctatgaaaatattcccctcagggaagatttttacatgctacaaagttaacattgctctagcctctcaatttaacaactactagttctaggagaaggagagcaatgtaaggaggaaggtgatgggagaagataatttagctgggagaactttctgcccagcagttttgaaatgttctttcatggaggcctttccaaaagctctagctccagctttgtggaagaagctttagttacctcttcttaagttaattgcagactggaaaatccagttcccatgtgaactatataaaacaggaagcctagtaatatcttcagagacctggggccacctgaatcaggaatgttgatgctattaaaagattactctagtagaggctgatggtgttcaggtggccccatgatcatcaggtagcataatattggggaagtaggattccggataaaatataggatgtatggaatatttgagacatactaaaaattacttcttgtttatctaaaattcaactggacatcctctattttatttgctaaatctggcacactatctggaagatagctccactttcctgtctccgtcttcttatcttctggcctgtagatgtctctgtcctggaggagaggtctagctgagagctcagggtcagagtctagggttgcatctaggtgctttgctggtggccaagcatgtccccaaggtagggtcaggcaaagcagaagttgcgtataaatctccatgttggattctgaggaactgatcatgaggttttccctcctgggtgaaagagaaagctcttccccaagtgagagacattttctcttagttgtcctttgccccaccctgtgacaggtctcctgtgtcatctggatgatgcatgcatcagcaatccttgccacaagggggcactgtgtgacaccaaccccctaaatgggcaatatatttgcacctgcccacaaggctacaaaggggctgactgcacagaagatgtggatgaatgtgccatgggtgagtaagcagaactttttctgttctgtgaagtgtctgttgagctcagcaaatgtttaaactgaagcacattttactgtagaagcaaattacatacacccaaacctcagattactcttgctacatggccagtgtttacaagtgacctttcagcatagccataaccatatattccattctggggattaactgtatgaaacgtatgtacttgattcttaatataaagccctacctcaatgacagtttgggcagaaactgcaatagcagaaatgcataacagttgaggatactccgcttttatttctttttaaattaaggaaaaaaaaatagaaaattgggaatgatattattgaaggcctggccctgatgctataaccatcagatgctcttctattgttcacatttgtggaaactcaactgctttgatcccagtaattggtacttgtgtctttcagccaatagcaatccttgtgagcatgcaggaaaatgtgtgaacacggatggcgccttccactgtgagtgtctgaagggttatgcaggacctcgttgtgagatggacatcaatgagtgccattcagacccctgccagaatgatgctacctgtctggataagattggaggcttcacatgtctgtgcatgccaggtaaatgggcccatcagcatgttgggtctagagaggagggactgttctgaacttctcttggtagagcattcagtatactattgcagtctgatgagctctgtcagcatttgatttcctttgctgactggggactagaggaagtcagggttcacaagcaaaccttccttactcaaagagtaaggcagtgctagaattcatcaagctatgtagaccaaattaaacaagcctgttatatcaaaatccacatgtaatagaaaatttcaaaaatgatttactgtaagaatcctttaaatagcaagttttcctgataccttcatgtaattacaaataccataccattccatttatataaataaattttatccacaattatataggaagtttccatttatctaaagtgcagtacatgtttagttgtattctggagctcttctgcatagaccatgtctttaattttgagaggatacagccaaatccagcttgaaatgatggctaacattggctgctctgcctgattaaggctgccggccactccagccttgaaaggcactgctgaccttgaactgcaccctgaaggcaacaacagctccagctcagcttgtctttaggaacatagagtggggacattttgggccttccacagtccagaaagtatggatttatcacagaaaatgtggagcagtccttgtttatatgctaggtcttgccaatgtcttttaatgtaatcccttcccgcttttcacagtgacatgggtatttcacagtatgccctgtcctgcttgacacttggagtgactatttctcatcatggcagagttccttttttctctgcagaaatgtgtgtactcgcaaaggatgtagttatcattgcaaatttgtgcttccttgtctggaatacacctttctctctctttaagccacttgtgtggtctgttttgtttaggtttcaaaggtgtgcattgtgaattagaaataaatgaatgtcagagcaacccttgtgtgaacaatgggcagtgtgtggataaagtcaatcgtttccagtgcctgtgtcctcctggtaagtgcccacgacctggccctgttttctcttaaagcacaaagccaactgaccacagggaagagagcagggagagaatgtgtgtgggctgtgcatgaggaaatcattagagtaagtgcttggtgataggagtgtgtgtgtacagaaatagagaatttgttttagctactatatcttacaagtcctttgtggccaaaagtccaagtctattcttgaatggcccatgacctcttcgtctgtgtatatatttcagaatgctcatacctcacagaagccttagttgtaggctattcgggattttatttttttcccatgggtcatattcttgaaggccctctgtgagactgttatttttagacatttcacccatgagttcagaaacagacaacacatgccaatttttccaagattctctgtctctccttttttttcctcaagctatttttgaagatatgccaaattgtcctccatagattgcatcttaccccattgtatcttcagagagaaggtttctgttgcctctgaagctacagttacttgtttgaggttccacctataaatttcacttcctacatccttcatttagaagtgaagtctgtctgtgaagaagcaatggtagagacacaccacaagtgcctctgattccctgagtgtcacctggttattccagttacctgttggggagaataataaagcataggcttattgacccattggtataagccctgctagtaacctaacatcattctcatggtaaagggagacacaagataatcctttgactcatgttaccactgtccctttttgaggtttcactgggccagtttgccagattgatattgatgactgttccagtactccgtgtctgaatggggcaaagtgtatcgatcacccgaatggctatgaatgccagtgtgccacaggtaagattcttctcacttttcatctccttggtaggtccatctgaacaaaatagggtgattgtccctgtcctctgctagctgctgtgccaggccagtccactcccagaggctcctctgtgtttaggaagagctgtttctaacaaattttttgctgctctctttttctgcagcacctgcctcctccaccacaacagtggtctggcccagatatttccatttggtatccatgtctttctgggacagattttggcagcagtaagtttccagctggctagattgactcatttaggaaagagaagaaatccagttgaaagctatttttcactggggcttctcagcaggaggcctggctctgtgtttcttgaagcaagtgactgaaagaaggcccagaaatttaaaggctgaatgtgtacaagtgcaggggtattctgcataaaagacaccaaaataggattgagagtcaagtgtgtaaatgacaggagagagagctctcactgggaaagtttgggtcctctttgctgacttctgcaactagagtcaaaaacgatctgtgcacaaatattctgcatttctgggtttgtaatcacattttgtacttgggattggatttcagtaagccagatgttccctattacacatttccttttacacaggcaaatattatacagaaaggctagaaattattagaagtgaacttctttattatctgactgcttgagaaagaagttatttatgtaattcaagacattaattttagagaatggagtcttaaagattaaaggcatcatgcatataatttatttctgatcacagaaaagcaaatttaattgtgaaaattaatacaaaaagttaatattttaattattagacctacttagtttaggacaactcagatgagccactttctttctttttgcaaatagttaatgcttgtcagagaatccagattttgtcttcccctggccagattcttcttgtagaggataagtttcaaagagattaaaaacctttagctcaggtttaaaagtggggggaggactcctggagtcttttagggaaagatataatgtttgtctatttacacctcgaagaaaggaaatcttacttgtctctttttcttttaaattctcataacaggttcattcattttattcaaccagtatttatttaaaactactatatatgcatactactctcagaggagttgagagaagtaaaataaagctctcttacctccaaagtcagaattggggacataagatacatgaaatcattaacctgtatattggacttgggaagaaagaaatacaaagataaatatataaaggaaaaatattttgagctgttatacctgaagcctagtaagaaatgaaggtgcagttaaagcatctctgcaatgaaattattgatcagttgcgaaggtatttcaattctaaaatttgatttggaaatctttattttaagcacctctctccaggcatactttctaaatcaatgcagatcgtcgttttctactgctcaagacctgcagtattaattgaccacacatcgaatgtgtagagtaaatggatgtagctacacttgtgttctggtgtggaatatctgccttggtctttgagaatccttgataaaattctttccaaaggtttcactggtgtgttgtgtgaggagaacattgacaactgtgaccccgatccttgccaccatggtcagtgtcaggatggtattgattcctacacctgcatctgcaatcccgggtacatgggcgccatctgcagtgaccagattgatgaatgttacagcagcccttgcctgaacgatggtcgctgcattgacctggtcaatggctaccagtgcaactgccagccaggcacgtcaggtaagcccactctgtttatgtttggtatggggttttcactgtttctcaaatctgtttacacagatgtttcagactgttggcatatgctcagatacctaaaaaacagtcctgattctgagaagcacagccaaagccacaaacataaatgtctcaaggcaaatggccctagccagtggagaagtcagagacaatagctctgccaatctgatgattttgtgtgtgtgtttgtgtgtgttttgttaaatagaggcataccacttcaaaaattaaaattaatgatttaaaattgaaaaccctcttttttgtggctatattatctttttttctttaagttccaccccacccagattaataaattcagtccttccttccttccttccttccttccttcctacctaccttccttcttcccttcttcccttcctttaatgtattttctctttcacagttcagtctctttattgtgacatgtttcttcagtcacttgtctctggtggggccttagccatcttgggggattctaaggaagaggagataccttacaacacaccacagtaactaaagtagaacgcagagctgtcatttcctatcctcggagttcatcattgttctctgaccttggttgaatgactccttgttggtcgtgaggacatgctgctcctttctggggggatgtggaattattttctcaggcagcagcatctcacaggtttggatggttctgttttacttattagatgaatgattatggtcacaactatggttagtggacagagatgtactatgttccagatcaaaggaaaatgtattagtggaagattaaccaatgtgtgcctttgttagaaggaaacctgccaatataaaggagtgatttattgagaactcattctggatgtaaggaaaatgacctatgtagagagcagaggtggagagggtagttagctaccctcattttcttttactgtttccaaatccatttcttcaaatttgtcaaaggctctggtctttacccaaggcttcatacatgccaagtttgaaaacaaagtcctttttgaattgctcaagcatacgaaaaacatcccatgccgtcacagctgaaaatgcctttccctccccacatttagataactcactaaaagggctgaatagatctttgtgaaaacttccaaaagcaatttgcttccgggctgttcctcagacatgagaaattatagtaggaaaagaaatttgagagatttacaagcaattgactggatgtctttagaatcgaggggctttagactttgctatcattcccatcataataaacatactggaaacctgggtgagtggtgggttggcaagcactttgttttctctggcattatatcattataccaggatatatcaggtgttttctggggaggacagagagggtatggtataagtagctgtcactgagaaggtgagaagtcaatatgagtgatctgtaggcagcatggaaaggaaggcccggaatctgtcagaacctgccaaagatgttcctgcagttatttggagaattaaagcaagcaacggtcagacaacattaaaaaaatgttgatctgtaatgggtggagaaagacctggcatgtatcagggacctgggtggaaaatccattaaaagaggcattatgaatagagtctgaagttctagagtaagaattcctgttccacttatatggcctaagttggtagtttggtgtttggataggggagtggatggaagaaatattttacagtttgtcagctaaaattgtggtagtcactcactgggtacttaaagtgagatatactacccagagttaccttcagtgcgttctctcatctgtgcttttatgatagtaaagttttaagtaagtttctttgtgtttcaagtaacagaccaacttgagctagtttaagtcatcatttacaaaaaataaacaacaacaaaacaaagaaacagaaagagcttagcgaaagcacccagagcctcttatagattccaagcaattgaataataaggctttggagaaaagaataggaaactgggcatttgtagaccagaggtcacacagtctgtgtttcagtgttgtaatgagagaatctcattggcctggcttgatccaggcatcttttcctactcaaataaaccatggacagggggctggagtcacattggatcatcagggcttcattgggagggggtgctgccttcttagtttgagggtaaagcagggttgtcagtcaagggttagaaacatctcctaaaggtatccactcctctgcagggaatatggaaatctttccctcatttatggctaattaggcagtatagcctcattaatcatgggatgacagtacagtgtggtggaaagaggatgctgtggagccacacaaacagggttcagagcccagctttcctgccctaatggtggcactgtgcaggtcagccctgttgtctgtaaaatgggtgtaacacagactaatgtgcagggtgggtgacagggttagagaaaacttacatgacaggcaaagcagcgtactttgctcatagaactcaataaactgttcctctgtaattattattaataaacattattgcagagtatgggcacagtggctcatacctgtaatctcaacattttgggaggccgaggtgggacgattgcttgcgcctgggagtttgagaccagcctgggcaacatagcaaaacctcgtccctacaaaaaataaataaaaattagccaggtaaggtggtacacacctgtaatcgggaggctgaggtggggggatagcttgagtcagggatattgaggttgcagggagctgtgctcgtgtcattgctctccagtctgggtgacagatcaggaccctatctctgaaaaaaaagaaagaaaaacaaagtatgagtgaatcacaagtatggcattttatcctggatgttttgttttcttatattctcattaccagtttttatttttagtctttttaaactaccaaaatattattctgtgatactcagaaagcatcactaaattcatacaaaaataaaaagtaaatattgttaccattatttgtaaagagatatttgtgaaatatattacaaatatatttcacaaatatctctttacaaataatggtaacaataatgattgtatactgttaatataaccaaactcttccattcatctgggcagtagagcaaaaataaggcagcttacctcaggaggaagttactagactcaagatttatcctgaaatttagcctacaatactgacaggcagtgtcaagctcacagtgatagatacaaattccaaaatcctaattttgatttgaaagcttaagttttatctttggcaaaaatactgtcaatttgcttttcctcaaaatgacaggtttgctgctttcacttaatatttgctaaatactcaattctgattaaccctggtttgcctgtcagttgctctttcaattaaaatggggtttcatgaagaaagcagctatttcaactcacagctcaaacaatagcattatagctcttcctggagacaactctcatacttggcatgccacagaaatgctttatgtatacttccatttcatcacatggagtagtagacattactcaaggttcgagatttattaaaattataaagttactaattttaacttccaacaaagccactcttgagtgaaactggcgtttttgcttgtttaactgtggcatgtggaggtgagcagtagaataattatagtacagtctggttctactgccttgattcatgctgatgtgatcggtttcaccccattgcctctgcaccatcagtttgaatgttagcacagtgaaaaggaaagatcatggctaagtatgattaataaaatcctttggacctcctgtatcttcctgaacgagtctgtggactgcattttgagaacttctgctttattctgtccagggttggctggaattcaccttagatttcttaccacgttgctgcatcacaagcagaaagccttagtatttctgtggtaacattgaatggtttccgttgggttttctcttccaggggttaattgtgaaattaattttgatgactgtgcaagtaacccttgtatccatggaatctgtatggatggcattaatcgctacagttgtgtctgctcaccaggattcacaggtaaagctccttttactgcaaggcccctccttcagccctatcactttgggaatatagcatccaaaggaatattgcttttcaagtgtttcctatttctttgtcactctggagggttagtagtctgtttctctgcttctctagatcagttttctgttcacagaccttttctaaactgaaacatacttttcattaattagagtttttcgattatgtgtttatttgctgccagaaaataattcctagacagaccataaattgcctttgctttgcttgagttacattgcttccaaagctatgtttattgattacaggtagtagtcaaggccttccgtaaagacatattcaaggtaaaatcttatgaaaatactgaatgcttgcttctgggtacctcctctttgacatactcaaggtagaattctgtgaaaatactgaatgcttatttctgggtacttcgtcttctgaaatattgttggatgaggtcagagcttggggagaccaccctaatttgggtagcatgagagagatctttctgcccaggaggaagaaagtaaatggctagaagaattttgaatataacttctcttaaagtatgaacagcctaggcatttacacatacacacatgtgcgtgtacatgcacacgcacacacagacacacacacacacacacacacacacatcttctctctctttacttgtcaatattacctttatcttttaccctcctgatcaaaaagtgctagaggctactactgcagaaacctgaggcccctcactgacacacctgagtttcttcttggcatcatcagaaggcacacactgtacattctgttcttcctggacagtgacacacagaagcagccactaacatgttaccctttaataatttccatgtggttgtttgtactcgaatgtgagaaacacccttcagagtttttgatagagtctgcagaaagcagccagagcaaagtgaagtgaatgtccaccattcatttgccaagaatgtactttgttctctatttcacaagaatttctcctatttggtatgattgtggacattagaaaggaaagtttgcacccaaatgggtcacagtgatccctgttcagtgaaagatcactgagaagcaaaatctgatgaaaactgtcatacccaatgcccagtgctgctgtagtagcctgatgttctgcatcctgaaacttgttactccaagatcaagtggcagtcgagtgaatacacaaaaatccctgctagataggataataggtacattgttgataagtgggtaatacaaagagaataggtaacatttatgaagaactaattttgagctaggcactaaattgtcaattgtcaccgacgttgagaatacctacagaataagaaacccagaacacagagatgttaagtaatgttaccagtgcacagcagccagaattacatcccaggcaatctgacccctgagcccatgctcattcccactacactggacatcctgattaggggcctgttgtgtgtgtgtgtgtgtgtgtgtgtgtgtgtgtgtgtgtgtgtgtgtgtgtgtgtttatataaggcttctactggactcatttttttcagaatgccaatgaatcatattctttctctttttaatattcagcaaaccaggaaggccaagaagggagaacataggctaaaaatattctaaaataaacctaaggtgtagcatgaaaccttataagcaagaaatggaatgttttggttactaggtggtcccctgttcatggaactgttgcctatgtctgagatgatttttttaatcagctgctaaaaacattagagatgactgtggtatatactcatgagtggtaaatgtgaggagttttattcttgctcttgttttattttaatatatttttcccatatcattctaataatagctttgacagctttaaaggggatttttttgttttaatactaagttatctgttctcttagaacctttttaaatgggggaatgtaaggaattatgtgatacttctataaactgcttttcagttttacaaagcaccttcatgcccattatcttcctgaattctcataacaatccagaaaggaagttctactatacccgtttaacaaataaggcaactgagagacagaccttgatgtatatactcatggcctctgccagtgagttgcatagtgaggactgtgagttcccagaaggcagagacctcgtttaatcatctttatagcaagtgtctaaagcaatgtctggcacgtaggaaacgcttagtgaatgttgctgaatgaacgaatgaatgggactcaaatatagcgcttttgctagctaatgccagactctgaagtgtggactataccatgttttcatttattaaacaccaatttattgagtgcctaagtcagaagaaacactgggtaacttctcattttagttacctagcccaaaaaatttcttggccttccactggactttcctcatgctcagtggaaactccttgagggtaggaccttcctgatggaacagctgtcaatcaacatagtggactgcatttaatgctttcagataaagatggcttgaaaatttattctgcaaagaataaatttaaaagcaaataaagaaaacttgggaggagtggcttctgaggagacgcttccaagaaaagaccaaaaatgaagaaacctttttcctgttatccttcactatattacgggccgaggctaactgtgtggttgaagagctctggttcatattttggtgccactgacaaacagctcaaagaatcagtggttgaggccaaggaaatagtatttaaaaataagctattaataaaacacttaagaggaaaacagcagccacttccagattatgggtttttaccacaagaaccaccaatgatggtttagagagcaatggagggtgaaaaaatgtgaatactcatacttcataggagtgggtatctgtttcatttggaatgtcctattctaaatacaggattgctctttcttaaatgagaaaaatgcatgttttcaaaatcagaatcagaacattgtgttctttgaaatatataaattcatagcagttgcttgatgccactcatttatagaattataggtaattttaacatttcatatcattttggaatatatagttaaatctcagttactcaaaatgtaacaaatggaaacacttgtataactgacccgtattactttattcagcctaagaaatatcaaggttatagcagacatctttttaaagagcaatcttggctatgtcttggaggagcaacatattcacttcaatttcacttactcccagtatccacattgtatgatgatgataagtaataattgtctagaacagaatttctcaaaatctagtttttacaccacctacatttaaaaaaatatggtttgctagattctattcatcacaatctatggtgatagggcctaggaatctccacttaaaataacaacttaaagtgatgattttgcacattaaagtttaagaaccataggtctgcagtgacagactgcattattttctctaatatcagaagacaacaaaattatactttgagactctacttcctgaggtcaaaaaaaatggattaatagactctaagaatgaaaacaaaataacctttctttacaatcaatttaattattgaattaagttactagcccattcattaagcattccagttaatttggatttaccataaatagaaatgtaagaggctgagaaaatgaatttgtgctaaaccggcacaaagcatgttaatgaatgcttctttgagttgtactcagggcagcattaagaagagaagccttctttggtaacatttgacattctcttcttagaaccttgtgaagtttccccatttctgagttgaaagaaaatgccaaattgctataacacttttccagccttttgttaactttccttgattatcaaaagtcaatgctctttgctcctttgcatggcattaagtgttattgggtgtctaattccttacatcttgacatttggtgcctcaaggcaccttatggcctgagcagatgaaggatgtcaacataacgtgtcttatagttctgggtctatttcctaagcatttaggatgtgactaacactgtcccccttctccacagggcagagatgtaacattgacattgatgagtgtgcctccaatccctgtcgcaagggtgcaacatgtatcaacggtgtgaatggtttccgctgtatatgccccgagggaccccatcaccccagctgctactcacaggtgaacgaatgcctgagcaatccctgcatccatggaaactgtactggaggtctcagtgggtgagtagctgccccatgtgatagtttcttattgacctgtgttaagccagttggcttgtagtactggtgacagcctgaagttgtcaaatggggctttgagctgaaactttgctgaagatttggtagtctgcatcaaagccttctgaattaccatttaatccatggaagctggcccctcccttccataagtgaggacctagatggaacataaggaggaatgtctcccttatatgggtcattagcaaggacctcatgggaggaacccatactcattgtatgaatctttggcagttatgtttttagtatgtattactggaatgatttctgaagaatgttctagcttttgattttttttcccctgcttaaacatagctcagctctgttctgtcacagtgccagtgtgtgctctgaagtagggccgcttcagccaactttagaaatgtccctgataaaacatttgcaatttttaagccccacagacttgtgtgatgtgttagtgattttctagaatcatagagtgaccttaggtctcacgagacgctcttatactcttgctgcatttgccataggatatggctttggacattttcctttgccaaactttgaaagatgaaaggataaagagtgtttagtggcaaatagtaaataaagttatttaaaaattaattaactcagtattggtgtttcagagtatagcagccttcaaggcacccttatccatctctcattttcttaaaattctcccgataagaaagctatcatttcctgatatcacattctaagtgttagtgatgttcaaattggagattatatttacaattcaaataccttcattcctgtgccttttcctgcagaataccctaggtgctttctgaagccaagtgttaggtcaagacaaagcatgacttagatatgaaatatcccaggagcatcttagagtggctcagggtccctgacagtgacctaggtatctatttggcaccaaaaactaatgatcttaatgtttgctttactttagttttatgtataataacatacattgtgcagatttatccatgatattgtttccttcagagtggcccaatatattgtagaaaccattgtttacataagaaatttcattataatttcactaacttagtgagccaggaaagcaccaagaaggtttctttactggatacacctgatagggccacatggtacacaccatgtgcaaggtgatgcaaggatagttccatgaaagtagaaatgagatcagtttaccccatttcttcaccaagtcctgtatttcactctatgactcaatcatacgtttatacctcctatagtaaaaagttttgtcttctttcagatataagtgtctctgtgatgcaggctgggttggcatcaactgtgaagtggacaaaaatgaatgcctttcgaatccatgccagaatggaggaacttgtgacaatctggtgaatggatacaggtgtacttgcaagaagggctttaaaggtgaaaacaaaagtacatctttctgcgtctgctctttgtcttcttgttgttgcatactgcttgactcacttcttagtttccttttctctttttcattcattcgtgtaacaaacatattgagcaattagacttgtaaggcacttggggaataaaaacatgaatagatacaatttctaccttcagcctagcagagaggtgacctattgaaatagtaattttttttttttttgacacagagttttgctcttgttgcccaggctggagtgcaatggtgcgtgatcttggctcactgcaagctctacctcccgggttcaagccattctcctgcctcagcctccctagtagttgggattacaggcatatgccaccacacccggctaattttgtatttttagtagagacggggtttctccatgttggttaggctggtcatgaactcctgacctcaggtgatctgcccacctcggcctctcaaagtgctgggattacaggcgtgagccaccgcacccagcctgaaatggtaatatttaagatgagcagttaagactccagttttacagcattggccaagtgtctaattataagcttcctggtccaggtttgagtctatgatagttcaaatagatatatggaagtagaatgaaaatattatgcttactataattaagatagtagtaatatcaaggaagaatccacatgttcagaactctttcaacaaactttttaacacattgagatattcttagttatttagtcatctttggtctacaccggaggtcctcaaccctcaggctacagaccagtacctgtgtgtggcctgttaggaaccaggccacacagcaggaggtgagcagccagtgagggagcaaagcttcatctgtatttatagccactccccgtcactcacattaccgcctgagctctgtctctgtcagatcagtggtggccttagattctcataggactgcgaaccctattgtgaactccacatgtgaaggatgtagattgtgcgctccttataagaatctagtgcctgatgatctgtcactgtctcccatcacctccagatgggactgtctagtggcaggaaaacaagctcagggctcccaccaattctacattatagtaagttgtaaaactacttcattgtatattacagtgtaatattataataataaaagtaaagtgcacaataaatgggatgtgcttgaatcatcatcctaaaatcatccccccaaccccggatcatggaaaaattgtcttccacgaaaccaatccctggtgccaaaaatgctgagggactgctggcctacccaacaaatggatagtcatgggaattgtgtaaaatctgcttctgagttatcactgttatatttctaccatcagttttctcttggaaactcacaaaagagagccgtgtgggtaaatcattatatttgcactcatctgtctatttattcattgatttcctcactcaaatattgtgctatgctattgtgatacatcagcatgttttagagccactttatttgattacttgaaacatttctgagactgtatttcagccattaattttctgatatattgtattacattggcaaagaaacttgtagatttcaaaacacttttatttctttatcttctgagacttgcttatcagtgaacaaaataaaattcaaaaggtaaaatcccaaattatatgcccagtgggcagcagaatactccttgaacccatacctctgcattcctagaccatgttttcttttccacagtgccatagtttctctactttaaggctctgtgccaggccctattggagatgcaaaggtgtgcagtatttgggtactgtgctatctccactaattttttattccttttatccttttcttagtgggagaaggtttcatgccttgaatcaggagaaaacagctttgtaacttcaggaagtaccacatgggtggtagacatggtgcctatgaaaaaatgggcataaaccagaggctttgattaatgtattcaaaaacatcatcccttttcctgaccagaaatgttatatacaaagagcttatgtttatcatgcaacaagctattggctgggtgcgatggctcacacctgtaatcccagcattttgggagggtaaggtgggaggatcacttggccacagaaattggagaccagcctaggcaacataacgagacctcatctctacaagaaatttaaaaaatcaagtgggcatgatggcatgcacctgtggttctagctactcaggaggctgaggtgggaggattgctgagcccgagaggtcgaggctgcagtgagccatgattacacaactgcactccagcctgaatgacaagagtgagaccccctgtctcaaaaataaataaataataaatagatcctgcaacaagcttttgaaccaagaacttcattattgagaagttatttgtagttggaggggcattaggctttcagatcattagaatagactgtttttccattcatcttttcaactaattgctgtcatttactgactgtatttcagggacatggggctgaaatgaatgaacaggtatttcagtggcctgaagaaatgaatgcaatttctgatgtgttcagtttttacccctaaagagaattggttagaaattgatgaggctgtcagaaagttttactgtttttggttttttccttttgtctacaggctataactgccaggtgaatattgatgaatgtgcctcaaatccatgcctgaaccaaggaacctgctttgatgacataagtggctacacttgccactgtgtgctgccatacacaggtgggtcctggaggtaccagcagggaccagaggggatacttgaccttgctttgtcagtgcctgagtctcagtgcccaggtctgtggggctgtaatgctcctccgagggctcactcctcagttgtccttcctgcctttactgactggatcttgagctttgaggaaacattgtggcctcctaagatgcaagtggtggacagtcatttgcctacattgtactttagtatttgaaagaaatttttttttaattgtatgtggctggaaccaggcacatagatgatggttgtctatcatttgaatgtttccaggacattagctatttgtcttagtttcaataatacagctaccacttattggtaatttactatgtgccagacactttttgtactttggattatttaatgtattcctcaataattctacaatgtagaagttcctattgccattttacacatgaggaggctgaggcaaagggagcttgcttgctcaaggtcatataggtaagtagtacatctaggatttacctgtaggtctctttgaatctgaagcttttcctcttaatggcctttttacttcccacattcagtaccgctacatctcactcagaggtgtcaccccttcgatggctgttacaactgagaagacgtgagtgtgactgcatgtcacatagctgttctttgtttcagttgttatgtttggagtgtgcttgggacatttattctttgttttctactggcttattatcgccttcttgtgattggttatatttctatggcttgtttatctcctcttcatctatttttctcattcagagggaacaaaacacaaacatagacaataactttgcctgtttcatcaggttgcggcatgcagtcaggagcatggctgagttattacttccctggagtcttgagctgttaatagcagctgttaatagtgactctaagttgtgtagagtagcagaatagccatagcatgactgtgttacctgaaagacttgatagcacattgttactttttcccctctagaagaaatagtaggccaggcacggtggctcaccctgtaatcccagcactttgggaggccgaggcaggcggatcacgaggtcaggagatcacagccatcctggctaacatggtgaaaccccgtctctactaaaaatataaaaaattagccgggcgtcgtggtgggtgcctgtagtcccagctactcgggaggcggaggcaggagaatggcatgaacctgggaggtggaggttgcagtgagccgagatcgcatcactgcactccagcctgagagtcagagtgagactccgtctcaaaaaaaaaaaaaaaaaaaaagaaatagtagtgtagggaaactacatttaacaggcctagttcataaaatcacagttgtaggaggtccctaactatcttgcccaaggttacacagttaacaatatcagagccaatgctggaaagatcatgggcttctccattttcaaagcctcttaatcaggacttttgacttgaattcagagaacatccttgggataaggaaaattttcgttttcttacctgtttatctgtcataaactctgtcatttactttctaggcaagaattgtcagacagtattggctccctgttccccaaacccttgtgagaatgctgctgtttgcaaagagtcaccaaattttgagagttatacttgcttgtgtgctcctggctggcaaggtaagaacatgggtgtggagaagccaagaacatgcattctgactttaaacaaagtggtcaggttatcagatcatatggaaggccctgctggtcttatgaggcctgtccatcttgtcaagagcacacaggttttgcaaaggagctgctacttgaaatggcttagaaagcctttccattgtccttgtcttcggggccccctagcggctggaaggctgcaacctaagcctcacaagccatgatgttcattcttcagggagtttaagcaccttggcagcaacttcctaggccacagaagtcttcctggaacttcctaaagcagggggccaaccagtttgtctgtggaatagctccatgcaccccaactaagctcaggtcctgcgtagatttttgtgggaatcagcttccaaggactaggtcatttattggccctgtgttcctaggtcagcggtgtaccattgacattgacgagtgtatctccaagccctgcatgaaccatggtctctgccataacacccagggcagctacatgtgtgaatgtccaccaggcttcagtggtatggactgtgaggaggacattgatgactgccttgccagtgagtatgccagtcagctcttaagcccccagaggagaggaaaacacacagggagcagaggagagcccagtggaggctgccgcctttcacgcctgacattgggcataccggtcatttatccagcatatttaacagtagtgttttaactctttcttttgaagctagtgtccttgggaattgaggcagtggaatatattttaagcattccctttaatttgtgaacattgaaaatgtgaggacaatgctatgatagtgttttttaaaaaacagatttattgaggtataattaaggtacattaaactgcaaatattttcagtgtacaatttggcacattttgacatatagtaagtgaaactatcacccaaaataataaacatatttgtcaccctaaaactatcttcatcccattgcaatttaaccttctctctttcttgcctgtggagtcacagttttaccttctgtcactatagattattagttggcagattttatcattttatataaatgcagttagataagcataagtcaggagaggttctgacttgttttaatgaacatagtatttgagatccgtccatgttgtttatatcaatagtacattctagtttattgttctgcagtattccattgtatggacatattacagttcattggttgatggccatttgggtgttttccaatttggggatattacaaatgaagttgctatgaatgagtctttgtgtggacatatggttttagttctcttgagtaaatacctaggagtagaatcggtgaatcatgtcgtaagtgtatgtttcactttttaagaaattgccagaatgttttccaaggtggttgtactattttcattctcaccagaattgtatgaaagttctgtttgaaccatatgctcacaaactcttggtgatatcatttaaatttagctcttctagtgggtgtagagtggtatttcattgtgattttaatttgtattattctctctgccaatgatgttaagcatcatttcgtgtgctttgcccatctgcctatcttcgttggtgaaatgtctatttaaattttttgcccatttgaaataattaggttgtcttcttgagttgaaagagttctttgtatttttaaataagaattctttgacaggtatttgcttcccaatgatttccctccaatttatagcttctcttttgattttcttttgaagaggaaaatgttaacattgatgaaattcagtgtatgcatgtttttcctttatagttcatgcttcatgtatcttgtttaacaaatcttcacctaatccaaatttgaggtttatctactgtggtttcttctaaaagttgtgtaattttagctcttacattttggcctatgatttgttttgagttaatttttacatattgtgtgacttaaaagtcaagtttaattttgttttcttactgtactaaactaacttcttattagttgtaggttcgtaggttagcctctagatagtttagattcttaggattttctacagatacaatcatcctgtctataaataacccttatgccttttaatttcttttcaaccctgtatgccttttatttattttagtattattgtactaagatcttcaataaatacagaatagaagtggtgagagaagccttaccttgtttttgatcctaggggaaaagtatgtagtcttttaccattaagtgagatgttacctataggttttcataaaggccctttatcaaattgagagaccttgtacttctggtttttaagaggtctttttgtatttttataatgaatgaatgttgattttttggtgtgcaattgatttctctacatctgttgacatggtgatattattttttggtcttttaatgtaagtaaattgtcatcaactttatttaacattttgcaaatgttaaaagctaatcttaaatttatgggctatattccacttggtcattatgtgttattccttttatgtattattggatttgatttgccaaaatgttgttaaggatttctatatctgtgttcatgaaggatattgttctccaggtttcttggactaccattgtctggttgtgataagagggcaatgctgtcctcatagaacatatcaggaagtgtttttctttctactttctgggagagtttgtgtagaactggtgtgttttcttccaaatatatagacatggaaccttctttctgggaaagtttttaactacaagtttggttttcctaatacatataagtgccatatgggctatctgtttttaattgagctttggtaatatctgtctttcaaagaattagtccaattcacctgttttgtcaaatttgttgacataacatcaatttgttttataacatttccttactgtgctttaatagtctattagatctgtgatggtttccttctcttgtttctactattggtaatttgtctcttctttctttgaaagggaagaatgtctgttttgcagatttttttcatacagtgttctataaatgttattaattcaaattggatagggtagtgttattcaggtcttttatgtcctctatgactttctgtctacttattattcatagaatataataaggaggctttctgtttacttcttattcaagagtattgaaatcttttactatcattgtatattcatctgtttctctagtaattctatttgttttggcttaatgaattttgaaactctaatcttaagtgcataaacatttagggttcttatgtccttttgaggaattgacccatttatcattttgaaatggcctttttaaactcttgtcatatttctctctctgaagtctactttgcctgatactgatatagctactctagctttattttattattagagtgtgtttttaaaaatccttttacttctggcctgtttctctaaaatgggtttcttgtagagagcgtatattgggtcttgtctttttttctccagtatgatggtgtctatcttttaattcaggtatttagactgtttaaatttaatgtagtttttatatggctggcttcaaaatatgccatcttgctgtttgctttatctttgttcttctgtgtattgtttccattttcctttttctgacatcttttgaaataattgagtatttttatgattacattttatctacatacataaatttcaaaactttatctatattcactaccatattggttacatattttttagtagttattgtagggtttactggtgtgcagctttaacttattataatgtaccttcaatcgtaccacttcatatagaggagaagagctgtacactcacatttacattcctcctggcctctcttctattgttgttataaaatgtatatagcattaaacccatcaaatatgtaattaaaaggtcaatttttttcaagatatttgaaaaataatttaaaagtctttaagttacctacgtattttcaagtcttattggtctttattcttttatgtagatttgtgtttccatcaggtatcattttccttctgtctgaaaaaatcccttgaatattttgtgtagtctttgtctcctggtgatgaagtctttctgtttttgtgtgtcagaaaggtttttattttgctttagttttcaaagatattttcacagggaggagaattctaaattgactttctctttcagcactttaaagctatcctattatcttcttccttgcattatttctgaggagaaatctgctgttattccacaattcttttatctgttccttcatatataattctctctcctgtggctgcttttaagatatcctctttccactgatttttaccaacttgattataatgcctgttggtgtggtggtcttttttttttttttttttttttagactgagtctcgctctgtcgcccaggctggagtgcagtggcgtgaacttggctcaatgcaagctccgcctcctgggtttgcgccattcttctgcctcagcctgccaagtagctgggactacaggtgcccgccaccacgcccggctgattttttgtatttttagtagagatggggtttcaccgtgttagccagggtggtcttgatctcctgatcttgttatccgtccacctggacctcccaaagtgctgggattacagcgtgagccaccatgcctagcctggtgtagtgttctttatgtttcttctgtttggagtttactgatatttttggttatgggtccaagtctggaaaatattttaaccattatttggtaaatatttagccattatttttttcaaaagtttttgcgtctcccttttataatgttcattatgcatttattaggctgcaaggttgatttatttatttttcagtgttttttccacttagtgttttattttggattattttactatttgaagttattttgttatactttcaagtttactaattgtttcctctatagtttctcgtttactgttcatctcatttagtatatttttccctcagatattatgtttttatatgtggaagtttaattcaagtctttcttttaacgttacctccctctttgtcatgttcatgcttttatttccttcttgaatatattgagtatcgttgttttctagttgataataattgaccatattttattgtccttacctacttttctaccatatgagaaattcctgagtatgtttctgttcatattttttctactcattttgaatgtatttcctgcttctttgcctgcctggtaactttttactggatgctagacatccatcttacattgttccatgctgaatttttttaaaaatcttttttaaaaaaacatttttgagctttgttcaaatattcaattatttggacatagttcaatccttcagagtcttacttcaaggtttgttaattggttccaaaatagtctttagttgagggctaatatggctcactactacaacaggggtccctcaggactctacttactgccacgtggtttttttcattcttattgaataaacatgagctgttctcagcccagtgaaacctctagggattattctgcctacttctttctggtggttcttcccccagccttaatagttttattacatgtatgtactgaccgatccttagttacttgtatagtgatttaacttcaaatgagattatcacaatgcattaataatggcaccttaatttacaggatatataacttcagattatcatatagaggccaccatgctaggtgccggcagtgataagataaataagttttagtttctattttcatgaagctcatagtctgttagggaagaaagacatatagacacagttcaagaccagcctggccacatggtgaaacctggtctgtactaaaaatacaaaaattagccaggcgtggtggcaggcacctgtaattccagctactcgggaggctgatgcacgagaatctcttgaacccgggaggcggaggttgcagtgagccaagattgtgccactgcactccagccggggcaacagagtgagacttcaccaaaaaaaaaaaaaaaaaaagacatatggacaaataattttaatgatgtattacaaatagtgaaaaagtatccaaggcacaattgatggagaactttagggagtgagatcaagctgtttgtattcctttctcacattaagtaacatgtcattcaaaatattgagtatttttaaatttttctgtatgaagtgggaccaacaatatttacaatgagtttaaaataattcagtaaattatggatggtgagtttcctgaaagatgctatggaaattatagtagctgtgtactgtggttaagaaaaactgaaacaatttttacaattgctttaatagagattgcagatgagtcttataagggaattagttcaagagatgaataaagtatattaaatgtttcgctaataaaataaaggtcacattaccctttagtttagtatttagttttcattttacatgtatgacctgtctttgcaattatatttttaagttccttaagagccatagctacagcttatttttcttgttctgatttgtatggcatataattgtagagtgccgtggcatgactgtagcttaaaatttgttggttccttactatgagtataaagtgttccaagatcaagctatgtgatatttcaatttttatttaaactgtgtttcataaggctactgaaatctaaaagagagagaaaaagagagagagaggagatggaaatgaaagggagagagaagattatataattaatatgcctggaaaaacatgaatatgtgtattttgtcctcccccacacactccacaatcacatataccttttgaaaatattgattgatattctgtgtattggggcacatatttgatttccaaatgtgtgagggaccaatgcaagttttatgactattaattttacaaaactgttggtggtgacatagatgaagtttattccaattaataaacaggttaaaaacttcaagcaagtattattgaattcattttcataaatgagggagttggcaaaattgaatgaaaaagtcagaattatgctgcccccagcatgacttagttcaggtcattcacttttatgaatctaagattaaactgatgctactcttcagaggtctaattcttttgtaatggcttagatccttgccagaatggaggttcctgtatggatggagtgaatactttctcctgcctctgccttccgggtttcactggggataagtgccagacagacatgaatgagtgtctgagtgaaccctgtaagaatggagggacctgctctgactacgtcaacagttacacttgcaagtgccaggcaggatttgatggagtccattgtgagaacaacatcaatgagtgcactgagaggtgagcaaccagtccctcgggcatcagcatcttacgagagaaagggagaaagggaaggaaatttcagagcacaatgctagaattgtggagcagggatgaggtgtccacatggatcccagtccacagtcatctcctattgcccggttactggttcctgataaacatatataatagaaagttaaaggtcagaggttgcaaagtttatagggatggtttattatttcttgcttagggaacagaaagagctataattttgttctgttcttcccttggcctctcattcatttcaaaaatccaaaaattccagtttttctgcagagattggaattgttactagtctttaagtaatttctgggagttttgtttcatttctttttataatattatgagcatgtttgttctatttgtcttttataaataatctatagtgaataggacttttcaaataaattaaattgacttagagataaaatgaaatgtgatacatgggaaaactacagaaggactgggtaaaacctagaaaacttcattttctgtaggtatctatagtaagctcagtcttttaaatacttctaagcctttgattatagaaaggaagtgggcacctcagagcttctggcacccaatctagcttgtaagaaagtttaagggatatgggttgctcagaggtatttgcctctgtatgatcttagattccagaaatcttactttgtagtagtaagtcacaggcaccagatacatagctgatgttgctctgtttgcttcttagctcctgtttcaatggtggcacatgtgttgatgggattaactccttctcttgcttgtgccctgtgggtttcactggatccttctgcctccatgagatcaatgaatgcagctctcatccatgcctgaatgagggaacgtgtgttgatggcctgggtacctaccgctgcagctgccccctgggctacactgggaaaaactgtcaggtaattgacttccttcccattcactcagctcccctagagcagctgaatattatacctgttcactttctagtctgaataagttccttctaaaagagaaaattgtggatctcagggattccctgagataatctaggtaacttcttaataacttgcagattccaaaatggtgctggcaatatgatatagattcacaaacataagatcttgaaaagaggctaaccaagaactgcagaaaaatacatgtgggaaataaacacagatttttgttttttgttttttgtttttttgttttttacattggaagcacaaaaagaactgaagaaaatagagaataatcaaaataaccttttaaaggtcagatgcaacatctaagtgtttaataatgataattgttgtatcttgcaatgtggtaacattctcaaaaccaaacaatttacatatcttttaagttcaaatatgtaaatacacacttccattaaattttagaatggaaagagcccttataggcaactaatctaaccttcgatttcatggaaggcgaaacagccccagaggggcaaagtaacttttccaaagttgcataccaaacagccctgtgtttaatttaatgttattctacctctttaatagtgatagaataataactccaaagaacataggaataaatggccaaacttaacttgacttacagggcgcacttaagtaaggcttaggctatattagacatgtgagcacccacctccacaaaattaagcagcttgttgctgattccatggatcacgagagcatttttggttgggctcctatttgttctcacagtgtagattggcagtgctggtgaagcactcctcccttcctccagcaaactgtctatattgtgaatttggccttcctctcctcaaggggccaagccactccgtgtgtgaattaagatgaaaggtgccagtccttgtgcactctttgcttcagatttttcataagtctgattgccttccttagaaataagctcttaaacggttttaggcttattaacaggcattaagtccttttacccatattcaccaaagatagatttggtcaagacccatgtggatttctcacacaggattagcacagaaagtatgaacctgtctgacagctttaactctttctttctagcattacagggtggctatacatgtaggcaaatcatgtctagagcattgggaatagcttaacctgttgggtttccctgaatttttaaacttttttggtaagaactttttaaatagaagacctaagaggaattagactacagcaggccctcaagtaactttgtttccttcggtgtcattttgttacaacattgagtgggggaaaaatctgtttttggccagagctgctatctgtgtggggtttgcatgttctccccatgtctgtgtgggttttctccagggactccagtttctttccacaactcaaagtcatgtatgttgggtgaagtggacatgtctacattgtcccattgtgccagaataggctctggccactcatgaacctgaactggagtaagtgagttagaaaataaatgaatggggccgggcacggtggctcacacctgtaatcccagcactttgggaggccgaggcaggtggatcacgaggtcaggagatcgagaccgtcctggctaacacggtgaaaccccgcctctactaaaaatacaaaaaattagccgggtgtggtagcgggtgcctgtagtcccagctactcgggaggctgaggcaggagaatggcgtgaacctgggaagcagagcttgcagttagcggagatagcgccactgcactccagcctgggcaacagagtgagactccgtctcaaaaaaaaaaaagaatgaatataccaattattataaaatgaaaacccataaagcctatgataatcatatgagtgcacaacaataaacaatgtggtacaaaaccacccagcaagcctgccatattggttagtgttgctttctgagctgtgtggtggtaagaggtgctgacggttttgctttgcaaacatttattccttgacttgacctgccaccactaggactgctattactcatggattcaccaaaaattggataaataattatcttgtttttattaatatttcttaaatgaatgtatagcttgtttattttaatgtttaatatcagaagtgggtgttaatttgaagtttagtgattttttttttaaccagaaataagtagaggaacttaactcttttttatatcaattagactgtggtaaaattggtttcgttatatatcgttgtccttaaagtcacagtttccaagaacctaatgatgacattaagtgaagacttaacggtacgtaaaaatggttaggtgtacctcggaacactgaatctagtttaagggctcttttactagatgaaggctctttttctttttaatcagtttgcttttctttgatttcttgtaacacaatataacttcttttcttcatttctagtatttgaattttgatgtatgtacacccctgcctctgtttcagaccctggtgaatctctgcagtcggtctccatgtaaaaacaaaggtacttgcgttcagaaaaaagcagagtcccagtgcctatgtccatctggatgggctggtgcctattgtgacgtgcccaatgtctcttgtgacatagcagcctccaggagaggtaagctttgctcccttgggctcaggtcattcactgcagtattgactgtccagtggcatttagagaaggtatcatttttttgtggaataagagggaagggcagatagtagtgggtgggcagggctctggggaaagggctcaagacagaaggtgtaagggtagaggaataggttcagagattcggagaagaatgtatttgaaatgaaactaaggtatattccactttcagggtttatttttaatatgcatctggtactgagggttgtgaagaactggctaagaataagattggatgtgtcagccagtttagatttacttgctcattggttgatttgaaccccatgtgtcacatgttcttctatgacatttctccctcactgggtttcccaggtgtgcttgttgaacacttgtgccagcactcaggtgtctgcatcaatgctggcaacacgcattactgtcagtgccccctgggctatactgggagctactgtgaggagcaactcgatgagtgtgcgtccaacccctgccagcacggggcaacatgcagtgacttcattggtggatacagatgcgaggtgaggacactgagcaagcagaggtcatggacctcccaggagattgtacaactgtttgagcactgatatattgaatttagctgcttgtatcttatattgatagtttatagatttttaccaatccccgtcataatgtttaccactgtttctttcagcctctctgtgcaataagttaacaggtccctagtatgatatttttataggcgtctggaaccatcccagctccttaggcttgcttttaggattcagagccaagagtcactaccagagtgcattttacaggcccacccaacatagcagtccacacttccctgaatagcagctgagataggaaaaactgctttgtggagtgtccctttggcccctgtcacctttgtatttagtcattaatcagtcaactagcaattttctctcaaaaaaaaaaaatgttgctcatcctgttccccatagcatcatgctaaacgttacatgtatatatttgaggaagtatgtgtcttcaaggtacatgtcactattttaatgtctgggctgataatggttagagcaagcaagatattagcaaaatgcctgagacattaatttgataccaattcaaaccatcaagcaacactgagaaaagctttgcttctctattgttatataaatgagccaacgatagcttctgtattttggcccctaggttgtttatttcttcacagcaggctggggctgggtctcattagctcaaaagcctaccagtgccaaacccaaattttgacacatctagttgttttaaacatagcccccaaataagcagatttttagctatttagagcttgcctgctttgcatagcctgtaaaactgcagccaaaatctgctagccatagtataagtaggacaagatataccctggggccatacagaccccaggctgatgctggcctttagagctctctgacccagagattcctgccactggctgttgagtgacatcacctagactccttctctgatccttctttcccagagtttacttgctgtcttccccttactcacctacccctctggaaggtctcatgctatttgggacttctcccaggtgcaaacctgtcaaagtgtccaaataaagccacctcacagccatatcttttttcttggtcagccctgaaatccttccaacccctacagccaccacagccagccagccagccccacctccctctacccctaagcctatctcaaccagtttctcacacatagatccaatttcctaaacctggagatcaataattgcatgtctgtactaccatccacatatgccttttgaccgagtcatagggtcctcatcacatacggaggaatgtgtattttaaagatgaggaaacctgtacccaggaggaacaaaaggtttgcacacaagtcaaatctgaaacatccttcattgtattcagtccttgccttccctttaaagtgcctttttcaactgaaagaggagtaggtaaatgccagtaagctgcaggttttctgtccctgcatagaatgattagcagagagtgtgatggttccagagaaaacatacaaaagctaaattaggaggtgggagacagtcacagaaatgttccagatctcactagctctcatggtctgtttccagtaatggtccaatatttcttgtcctttttgtttcctcatggcagtgtgtcccaggctatcagggtgtcaactgtgagtatgaagtggatgagtgccagaatcagccctgccagaatggaggcacctgtattgaccttgtgaaccatttcaagtgctcttgcccaccaggcactcggggtatgaaatcatccttatccattttccatccagggcattgtcttaagttataaatccattcttagtgttcaggggattttataaaattaaagataggaagactagcttcattccaagcatttagttctacatcctagtaattcaagccattttattctcccatctcttgctagctctgatgttgtggtttatgttgtcagttttatctggttgtttggcatcttgatattccatgaaacacagaatatggaagggatacaacattagcataacattaaaaaattagcctggtcagtaagatttcttgttgcttcacagaaaagcaactaatggcctctaaaataaacaatttacatttacctgcttttgtatttttttaattagccaagtcttacctctataaggtgggggaatgaatgaaatgaaaatatctgcctattttcatagcttcaggggaggatactgatggtgtaaggttctaccttaggatgcagtaaggcctgatgagagatgcaaccctggactgttgagtgatggggccatcattcttctgtgggcattgccactgcttttgaactgagtggtatggccatcattctgctgtggacattgacactgcttttgaaccatcccttggaaagagctttgagtattgtttgttagaagggatgtagttatgctggggcctactctgagaaggctgtgaaaaaaatgtaaaaggccatccacactggtgagaaattcatccttgttagtgtcaaagagaatagaatgccagagaagtatttgtattgccttagagctttgtgtcagtaaagtttgataatttgccaccccaggtatcagagtttgactaaattaaaataggagagtaccctagtagtaggctgattattaaggaatgaggttaatgggaagaagaaaactaaggagaaacagtctttaagtatttgaagaattgtcctgtaaaaaggagaattgttctttattctagaaatcagtggttgagtagaaattaattccagttcattgttagtttgttcctttgagaaactgtactggttaggacaagctacgattctggtgatagctgttatcgtagttcatttatgttgctataaaagaatacctgagactaagtaatttatttttcgaaagaggtatatttggctcgcagttctataggctgtacaggaagcacggtgccagcatctgcctctggtgtgagcgtcaggctgtttccactcatggtgaaaagtgaagaagatccagggacccaaacacctcccattaggccccacctccaacattggagatcacattttaacatgtggtttggtgcagtcaaacttcaagctatagcagctaattaaacctcaaatctcagagcttaatataacactgattttgtcactcaaataaagtcgaatatgtgttgggctacctttctctgtgttttagatatgtggtttgaaacacctggcctccaaggcctccacagtacagggaagagggacagacttgaagggtcatacagcagcttccactagctcagtctggaagcaacacatatcactatccctcgcagtatattgaccggaattattaaatggtctctgtgacttcaagggaggctgggaaatataggagagtacatgagtattcatcaaactctaaaatcactactataccatgaacttttaattaataagacatgtccagtcctagaatggatcatttcagaaggaaacaaaatttttgtctcttctaaccatatggtaaagatggcttagaatactagttttgatattatcattgacagtctttttctttcagcactttgaatatatcatcccactgctttctgatatccatggtttttaatgagaaatatgctgttaatcttattgaggttcccttatatgtgatacattgcttctttcttgatgccttcaagattctttatcttttgatagtttgattatgatgtatgtaggtatgaatttctttgagtttatcctacttacagttccttggattattttactgtgtgtgcttgtttaggtatgtggaacagtatttttttatcctgacttaacttctctggccattggtaaacattccaggaaactctcaagtcccataaccccattgttagagtcatcacaatgtgtgttttggtcaccatatttctaagccttagatctttgcctgcatgtcccaccagtttcccaggtcaagggctaaatgtaacagttctgcctcttcctactactttgcttcaggctgaggttgagttctctctcagttgatagatctgtttatttctggtggttccattctgatggggaaatatgcatttatgctcattttactagcaatccctaaatatcaaatattctttgagacagggagcatatactagattttgtttgtttttacttttttgttttcaatttactctatgtataaggtgatatatgcatatatgcatatagaccaaaagtgtattcacacatagaaaagtatacagtgaaaatacttctcattcccatctctcttctggtgggcaaattttccctctcctgcaaacatgtaatcgccacttttggttgtttgtgtttctttctagaatttttcaaaaactgtatccttcagaatttctttatgtagagagaagccaaatgcaaagatattttatttcctttactttttactctgaagatagaatattagacacattttttgcacttgatttttttttacttaattggagatattttcatatcaaaatatagaacttcctctctctctctctgagatagatagatagatagatagatagatagatagatagatagatatttgtttgtttgtttgttttgagatggagttttgctcttgttgcccaggctggagtgcaatggtatgatctcagctcaccgcaacctccgcctcctgggttcgagtgattttcctgcctcagcgccctgagtagctgggattacaggcatgtgccaccatgcctggctgattttgtatttttaatagagacagggtttctccatgttggtcaggctggtcttgaactcctggcctcaagcaatccacccgcctcagcctcccaaagtgctgggatgacaggcgtgagccaccacacccagcctgaacttcctcaatattttttatagctacagaattcccttttacaggcataccatattttaaccagtgccatcatgatagatatttcagttatcttgcaggcatataagtatattggtgaaataaattttcagaaatagaatcgctggattggataatatgtgcatttatcattttaatcagtgattgcagtatatcagcatattggctttttcttaaggtggagaatatattttcatttaagaatcattttcttttgattttttttgcagtttctttgtttatatatatatatatgtatatatacatatatatatatatacacacatatatcttttttcccacattaagttgttgatgtaatgttttgtggaaattggctatgatataaaaaaacaaatatattcccagtatttttttatcttttttctttatagtgatttttagtgcacacttttactttttaatcaagttgaatttatcacttttaaaatggcttctggattttgagttgtagttagcaacatcatatacacttgaagatcgtaaagaaattctcccatattttcttcaagtacttttgccatatgactactcatttgttaatacacaatttattgaatattttgttttttatatatataatgatttgagatggcacctttgccagattctaaattcccatgtgtatttttggtgttcttctggattttctttattttctgtgttattctattaattcaccaataacgttctatttcaattattaaggatcttcttcttcatagttttcctggttattttttttcacataattttataatcagcttgtctgttaataaattcctgttgataatttttagtggggtctcattaaatttatgaattatagtatagagaaaactgataactttatgatgaggagtcttgctaaccaaatcctggtatgtctttccatttattcaagtctcttttgtgtctgttaggacagtattaaagttgtcttcatttaggtgtaacacatttcttgtgaagattgttcctaggtattttatctttggtaatttggtaattgcttttgaaaattgggccttactttacatttaaaaaaaaaaatcttgcttttgttagtgtatgctacaatgcaagtggcaggaatacatacagaactgttttcttttgtaccttttttttttttttttttaagacagagtcttgctctgttgcccaggctggagtgcagtagcgcaatctcggcttactgcaagctccacctcccaggttcatgccattctcctgcctcagcctcccgagtagctgggactacaagcgcccgccaccatgcctggctaattttttttttgtattttttagtggagacggggtttcaccatgttagccaggatggtctcgatctcctgacctcgtgatccacctgccttggcctcccagagtgctgggattacaggcttgagccactgcacccagcctgtacctttttttttaattatgattattattattttttgagacagagtcttgctctgttgcccaggctggagtgcagtggcataacctcggctcactgcaagcttcatctcccggtttcaagtgattctcctgcctcagcctcccaagtagctgagattacagacatgtaccaccacacgcagctaatttttgtatatttagtagagatggggtttcactgtgttgggcagattggtcttgaactcctgacctcaagtgatccgcctgccttagcctcccaaagtgctgggattacaggtgtgagccactgtgcccagcctcttttgtgcctttttgaattaatcatagaataatttctagaatagtcattgatttcttttgtttctttgtaagacactatatgcagatattcatactagatggctcatttacatctatagtacaataaattagtcctgttttaaatggaataacttttgaactctcagcaggaaatttatcctctatttttgttaaataaaaagcattggtgcacttatacactattacagaaaaaagccagtgattataaaacttaaaaatgtatttcattcaaaattcagttccagttgttaccaatatttttacataacctatgtatagtcatgggtcacttaacagtggggatatgttctgagaaatatgcccttaggcaatttcattgttgtacaatcatcatggagtgtaattacacaaacagagagagtatggcttactacacacctaggctccaaacctgcaccttgttttactgaactgaatattgtaggcaattataacacaatggtaagtatttatgtgtctgaacatatttaagaagaggtacagtagaagatttaaaaggtacagtaaaaatacaatataaaagataaaaaatagtacacctgtatagggcatttactatgaaaggagcttgcaagactggaacttgctgggtgagtcagtgaatggtaagtgaatgtgaaggcctaggacattactgtacactactgcagactttgtaaatactgtacacctaagctacactgaatttatttaaaaagtaattgtgctatgacattacaaggctatgatgttactaggcaacaggaatttgtcagcttcagcttcattatgggaccactgtcgtatgtgctgcctgcattgactaaaatgtcattatgcggttgcataactgtatttattgttatgttcttttttggactgtttttatattttggattatactttggaaaaattactgagcctctcttgagctccagtttccttctctttgagatgggacaaataataggacctacaccatagggtgctacttcactggattgtgtgggagagtacatgcggagccctagtatgttctttcctttacttgtacaactcaccaggatgacagctgtcatgaataaagagtgtgctttgccctgcccaactacaaggtcccactaccagttgaggagtatactctgattcattgtgaaaatgccaacaagggtggttatcccttgctggaaagcaggttaaaaggattttcataattctgtacctcttctctttcctccaggcctactctgtgaagagaacattgatgactgtgcccggggtccccattgccttaatggtggtcagtgcatggataggattggaggctacagttgtcgctgcttgcctggctttgctggggagcgttgtgagggagacatcaacgagtgcctctccaacccctgcagctctgagggcagcctggactgtatacagctcaccaatgactacctgtgtgtttgccgtagtgcctttactggtgagaagctctctagcctcctggccactctgccttggaggatggttttagtgaacaaattgggccctgtgagttgtcccagacccaagtgaagcttttcttatataagaaaaacaacagcagcttaattttttaaagccagttttttcttagtctgtactttatggatgttatgatcagtgtctacctttaatttttcctaagagattcttttgttgtgcattgattatatcatgctatggcaggaatattttttacaatgtatattgaaagtagttgaacaaaataacattttttaatagtggcaaaatatccaaaacctaaaatttactatctgaatcattttaaagtgtacagtgtacagttcagtggcattgtttagtacattgacattgttgttctactatcacccccattcattcacagaactcttttcatctgaacaaaataacattttgttttaaggaaccttacgtctaactccggtttcttcaccctgcctcctttgacatgtgatggcttgaattactgggtagttcctgccaccatgatctttccacccctaggactcagagaagcttaggaccctgcttagatacttaaagggggcaggtccaaattcaagtgtgtttaaccaaagatcagttgatgtgggtatgaaatgaaaagttgcagtggtcacaagtaatgccttgcccagtaaaggcttggccatgttcctactctgttcccaagcttctttctttcatcacagggcctgaagatgaccaagctctgcctcagaataggggtcttatcacaagaacttactagttacttctggagagggcagcatggtgacttttgggaactaaaatagttgtttgtatccagatgtggcaggatactcagatttgaaatttatcatgcatttcctggggtctgtagttgtatttcttatatggagggtgtgatcagattccttaggagtcagaaaataggctttaatagtctgtttacaaacaattcatcaagctgaactctgctacatcctgagcccatctccagccatctttccttccaactttaaagatatgctaccttacaatcttggaattttgtaaagaatgggagggattctgcaatatggaagctccctggatacactaacatcaaaaaagaaatagtccattgtgaacataatagctattgaggcaatgtggaacagaataagtatgaataatctgtctacagcctcgtattggccaaactgcactttgaaagccaaagcatcaaaactggctcctgtttctttcattgcccctgaggcttttttctctggaaacacaagcatttgcaaagtaagggggaagcaggagaaggaaggaatggagaccactttgaagtagtatcccacagccaggaagtagtgtttgtgcattttccacatttgtgaatgaaaaaaaaaaaatcccttgaatacattgagcacagttattgtgggatgctgttgcagagataaacagcaagggactggtggtccaccaagctgtgtccatgtggtggctgctcttggttagcggctgctgctgtgatttgaagagacaaacatttatacctcttgctgcttgatctcgttggccaaaaacattactatttttatttcatttttaaagctacctcttctttcccaagtgaccaccggccaaatggggcctatctcctgctgttggccaggagtcccagctgtgccctgcgtctcctctgtggttccagttgcctcgttctttatcttggagcagtggcccccaggggccttccgttttagaactattcctagaggaaactaactctaggaaaattaagctgtgttttaaggaatacgagttaggttacagaaaggccaaaaagtgattgaaatgaggtatcacagatgaaaattctctataccgcaaatcaccatttaaatgtattggcctggtgtagcctctcctatgaagtagataacattaacaaaatgcagtgaatagtttttcaactgtttttttgcaacttatgaaagctaaaatgacttcaaaaagctgaagtgtgatttctctttttttgatgggatttaagtcacttttctcacccaccctgttgtgtgagttggtacttactgtgtaggcaagacagagtaagggactggcctgagctggtgacaggccaatcatgttgagatgcagtgagtgttttctttctatgctcagattaccacaggcctagaactgaactctctcacacttgcttcacctatggctagttcccatcccagcaaagtttgttgagtaacttgcagtgactacttctgccttttaaaaccttttttattttttattttctgaggccggcactgtgaaaccttcgtcgatgtgtgtccccagatgccctgcctgaatggagggacttgtgctgtggccagtaacatgcctgatggtttcatttgccgttgtcccccggtaagtgccctgttgctcatctaagaaggggcattgtctcttccatacaatctgtgaacagaactgagattaaacctggttttaccaatcttcgaaatgggaagatttctcaattttagctctttcagaggaaagtgcttgggtttttccatttaaagctgtttacaagtgacaaaacaaaacaaaaacaaataaaagctgtttagacactcccagaaacagtgagcttcccaatccatgactgtcagtcttaagaaaaactaagggaaaaccacaaaaagtggtggagtgcccccttgaaaatagggctgatgtgtggggcttcttggccccaggctggagggtaatctcactgcccagattgggccagagttggtggaaatgctgctcttacattctttagcaggaaccatttggaagcaagttgtctcttaaagtagagggaagctcaattttagctatttgctatacccattcttttatcactgccagaggttgaaaccttcccaaaacaagagatccctatgcctcgggagataccaaaaggaaacctgtctagtaaccttggcttgggctgaacgggaagtacagaagggagtgagatgtggaggttttctagaaacctcaaacttccaatatgactgttttcctaccaatgccatttttatttcctcactgatttccacagggattttccggggcaaggtgccagagcagctgtggacaagtgaaatgtaggaagggggagcagtgtgtgcacaccgcctctggaccccgctgcttctgccccagtccccgggactgcgagtcaggctgtgccagtagcccctgccagcacgggggcagctgccaccctcagcgccagcctccttattactcctgccagtgtgccccaccattctcgggtagccgctgtgaactctacacggcaccccccagcacccctcctgccacctgtctgagccagtattgtgccgacaaagctcgggatggcgtctgtgatgaggcctgcaacagccatgcctgccagtgggatgggggtgactgttctctcaccatggagaacccctgggccaactgctcctccccacttccctgctgggattatatcaacaaccagtgtgatgagctgtgcaacacggtcgagtgcctgtttgacaactttgaatgccaggggaacagcaagacatgcaagtaagggcttcacgttttccagaactgaaggggactgtcactaacacacttcagtttctcgttcttgctcctaaccactccagtgcttcagcctaagctgcctctgcttttcagatggtgccagccttgtcccatcgcacactgccctgtgctctcccttgctgacaatgggccaggaatgctcattgcataatgaccctctctctctgcctatctttgccttctcacctgctgccttcgtgacctcctgggggatataacttttggaagtggtgtttcccacttcaagcagcctctcacataacctgcctgcaggatggatggaaaggtctgtatctctctcttcaccccattcctttccctaagactgtttttccccatctagatttatcaggaagcatgaattagattaagtctgtgaaccctcccggtcttaattgtataggatgctctatggtttacttaggaactttcctattaactagccattccatataatttaatcccaattaattctcagcaactcacgaggcataggtattactaacattctttcataggtgagagaacttaggcccaagggttaaagaacttgccaaagcaagttgtttctttgactcctaatcttctgattgagctagcatgaaaagttgtaccaaatagtataaatatcaaggggtaggggatgaggagaggagagtatagatggaatagtttgtacatcatttaggtttgggggttctaattccttttccaaatattgaaagagcctgtacaagtaatatctggtgtgctataaggacatacccatctgcctttaccttctgtagttgggattcactcttgaaagggaaagaaatgggtcatgttaatattgttctctgtatggcaactctcaatggttcagggccttgtatcaagtttctagaggcatctttcacttcctaaagcccgttgttaccttactgggggcaatggatttctctaaagtagaggaagcatgaaccaggttgacttttgtttcagatatgcagctttgtttgaaaacctgacttaacaggcttgtaaactgcctgggagcccctaagctcctgcagcatagtgagtacaatgttcttcatttaggaagtactcaacaaggatgagtggcataggcatcagggataggagatgcaaagaaggttttgtggggcatttgggtcctgggtgtgggccacagaaaatgggtaggaaaatccacagtagttccaaatgaaaaagcaaatagagctccagtctaatctgagctcttttgcctgttctctgcttccccttacctaggtatgacaaatactgtgcagaccacttcaaagacaaccactgtgaccaggggtgcaacagtgaggagtgtggttgggatgggctggactgtgctgctgaccaacctgagaacctggcagaaggtaccctggttattgtggtattgatgccacctgaacaactgctccaggatgctcgcagcttcttgcgggcactgggtaccctgctccacaccaacctgcgcattaagcgggactcccagggggaactcatggtgtacccctattatggtgagaagtcagctgctatgaagaaacagaggatgacacgcagatcccttcctggtgaacaagaacaggaggtggctgggtaggtgtttggtttctgaacttcaaggctaattttatgaagcctagcactttgacatatagcacaagtcataactcaacctatagaaatgcaaagatcagaaaaatagaatggcccaccacttaagataaacccgttaccccagtacttacgtttaaatgtaaagatatgccttcgtcttggctgaaaagtgggcaggggtacacaggaggcagaatgtctggaaggcctcctcagatgccagaatccataaccttttgccagagttgcctcaaatccctttattagtgacattcctttttacattaatcattgccttattgttaaggcccttctttagaatatatgtctctttgacacaggtaatgatcttcagttgctctgtcattcacacctcactctttgttggtatattgtcaaactttcaaccctccagctagctgagataccaatgcagagacaaaggaaaataaggactaaaaacacacagggatgtatgtttagagtagcaccaagtaccactgagtaggtatgaaagagtggaagaagaccactgaaaccttcgggaggaaagcacagcttctcactttgtcatgcattgcctcagccagaaggctttggtcagccagactaaggaaggtgggacatcctctatgtgggaattagtgtttaagatctcaagggacagtattctaattcctgagatatgtcttaaggcagcatcccagaggtggggacctgatgctgaaggggaggaagggaggctcctgtcaccctttatgacatgttctgcctgacctgcactcttctgttttacccccatctctcctcctccgcttctcccttggcatcccacagctctaaagtctttctggaaattgacaaccgccagtgtgttcaagactcagaccactgcttcaagaacacggatgcagcagcagctctcctggcctctcacgccatacaggggaccctgtcataccctcttgtgtctgtcgtcagtgagtggcactggtgtaaaggggaaaggtggcgggggaagagtgtcaattgggggaacaattttcattatgtagccctctattttttcaggactgttgaggtcaaagttgccctctaaatgaatctaatgctgacattgagaggttaatgttttattcagcactttccctgtctctgtggactttcaggtgaatccctgactccagaacgcactcagctcctctatctccttgctgttgctgttgtcatcattctgtttattattctgctgggggtaatcatggcaaaacgaaagcgtaagcatggctctctctggctgcctgaaggtttcactcttcgccgagatgcaagcaatcacaagcgtcgtgagccagtgggacaggatgctgtggggctgaagtaagaggcattgccaataagccactattcacagtataagtagggatgactagaaaagcatatcttgttgaatcatgattttaaattctaagctctgggcatttactcacattgaccatgattctaaatgccttgtgagtgactgaaaccacagtcagagcgtgggtgcatctattctgtgtatattatcacaatgccgcccagcttctgtgttgaggtgcccttttgttgtaagctcacgatcaatgggaagggaaaggatgaatgccaagggaatgtcaccaaaaatactgcccatcttctctgggccaagaccctgcaaaattgtaatatagccacgggagtgtgtcatggtggaaagtgttgaaaaccactgtattagtttatgactgccattcttattttttctttttataatcatattagaaatctctcagtgcaagtctcagaagctaacctaattggtactggaacaagtgaacactgggtcgatgatgaagggccccagccaaagaaagtaaaggtgaggtggccatggtagaaccaccattgtcagccattatctggttacatttcaaatttctgtcctttagctccagagtgtcctgaatgggagtaaagagtgatagtcttgatgtcttacccagacaataactatcctaggttctcctaaacagcaggaatccttaggacgtaaatgaccttgggttacctatatatgctcttcaagaataactctttaaaaatactaagcacatctgtttcctttagtgttatgctggcagtagtcaagtcaggaatggtgtgtgtagatggcaattccttcatggccacatctcttccttatcctttatgggcacttcttctataagagaagatgaaattaagcagtggaagaaagccagttatcattgacattggcaaaatgaaagccagtgatcatggcaaaacgttgacattggaggctccataccttgagcccaggaaactaaagccatataaaagcaagtggtagattgggctgagactgacatgaaaaagcagaccatccatccacagctgtgcatggctgggagctggtggagcatgatggttaagaagattgctttagttaatatcagaactggattcaaatccaaaactctgccgcttagtagctggcaagttacttaacctctttgtaattatccttccttgtaaaatgcggataataataaccctttcctctgtgagttgttgtgttggcatatgtgccttatctactttcctgtcatgtactaggtattcagtaaagggtagctgcttttaaattatcatgattattattattgaatagtctttcctgagtgcttactacatgttgggtgctatgttgagtacaaggaaacaaccttgaacaaggcttattttcccttcttgagaagcttacagccttgtggtggagatggccatgtagtgctgtgtaagcatttaacacatgatcagttaagcagaggtaagcagagtatgctttttagggccaacaagaagaattaaagtttactttctagtggagggtaggaaaagtgagaattgggtgctctggagatctaaaggctaaagcctagagtttgggattgggggagggagttgtctagatgatgcaggaaaggtaagcaggagccacatcacgagagcagtgggaagccaaggctgaggtaatcctctggaggggtggaggagtaggaagagagaaaacaatgggagataagcagcggtggtggaggctctggacaccttctgattagcagtttctggtggtggccagattgatagggagcattgttttcacctttcaggctgaagatgaggccttactctcagaagaagatgaccccattgatcgacggccatggacacagcagcaccttgaagctgcagacatccgtaggacaccatcgctggctctcacccctcctcaggcagagcaggaggtggatgtgttagatgtgaatgtccgtggcccaggtcagcaccgggcctcttcccctcttcactgggctgtgtctgcccatggttccacattgttgtccctgtgtttgccctgtccagtcataaaccccttcccaatttcacagactctaaatcagctcgaggaaatgaatgcttaaggaatgtagccacaaagaaattatcagctcagaaagggaaggatttcacattgcttcatgtacagtgtgttgagactaaacaggctgataaaatgggttagattgtagtgggatagaaaaataatatcccaaagcagaggatagttagtatcttatgcatgtattaattgttctattaatattagatatttttaaatgtccaattaaaataacagcacttaaacttgaacagaaaaacgtgtttagagaagagcccccagcttaaaaagacttgtactttcccattattctttacagaaatgatactatcagaggaaaaggtggcaaatgtgaaactaataaccagttactacaagaagtacagggaattctttgcccctaagaataactttagtcaaatagagctgtttcaaccatagggttaactaccttctccttcttgagtactttatggaattttccattcctacagatggctgcaccccattgatgttggcttctctccgaggaggcagctcagatttgagtgatgaagatgaagatgcagaggactcttctgctaacatcatcacagacttggtctaccagggtgccagcctccaggcccagacagaccggactggtgagatggccctgcaccttgcagcccgctactcacgggctgatgctgccaagcgtctcctggatgcaggtgcagatgccaatgcccaggacaacatgggccgctgtccactccatgctgcagtggcagctgatgcccaaggtgtcttccaggtaaagaacatgagaagagtcacttgaataataattcctatggctctataatgttttatcgtttttaaagaattaatatctcattgaatttattcaatcatttgagagattagggcaggtattagagtcatgcttggtagatgggaaagtgggaaaagggaggcaggcaaattgacctagctatatataggaagatagtgatagagccaactgcagacaacaccagtctttctgattattagtgataatttgttgtgccaagatgcctcccaaatgcgtagattagatgagagtgatagctggtccggagcctttgactagtatccgctacttcttatggattggcctgactgtatgggcttctagctgctcattggcagttttcttgacctgagttcatattatattccctgatgcctgtttatccttatgaggaaattggaagcattctgaatcaccttaggctgtatcctgactgaagttgtgatatttccccacatccctcttctttcctgagaaagtagaaggctcatcactttacatcagccattatttattgagcatgtatttatttattcatgcaacaaatatttattgattgatcatgatagaatcttgggcacaaagataacaagactttccctctcctccaggaatctacactgtagcctcagctctgatgcccttgtttcttcactgagtttcattattttccttcatccaggtgactctcttttctgtactttctcctcttctagattctgattcgcaaccgagtaactgatctagatgccaggatgaatgatggtactacacccctgatcctggctgcccgcctggctgtggagggaatggtggcagaactgatcaactgccaagcggatgtgaatgcagtggatgaccatggtagggatgaaaggcagggatttggattcttacctgcaaagcatgcctctgacgaggggaatagagttacgtgacccttagagaactgcctgagatccaaatacgtgagagattcactcattctttcattcatgcatttatttaacatgctgagctcagagatccaagccagtaggagatccctaattaaaatgagggatttttaatgagggttcttggacctcagagcagatacatgaaaggtgttcccatgtagtttcctaggcataacacttgatttgaggatttatgagagctgacttctctaggagagttgtagaatattctcaatataaattctgacatgtaatatataggtttttatcttcagtatgtaacagggtagattctgccatgtaggggacatgaatgcctcatacaagtgatcctttcacctagaggtcttctgcaggctaggcagcctgtactgaagtatttctagaacagaggaaaaagaagagattactttggtttttcctatagacccctacagagaactgcttcctgctgatctctggggagtttaagatttcattgtgcaagtcatagtgtcttatactaagagtaggatatgtctgtttataaaaatgtttctcccctgcaggaaaatctgctcttcactgggcagctgctgtcaataatgtggaggcaactcttttgttgttgaaaaatggggccaaccgagacatgcaggacaacaaggtacagtttgtgggctctgagcttgaggaggctcatagcagtgcctcagttataagcccattcccgtttctccttacagcttctcttgttatatgtatagacaaagatggaagcagtttgggcagacactgggtttcgagagcttgatgaggatattgggcttgtccaggcatgtgtcaggatggagttctctgccctctcagtactcagcttgcagtaggctggctatagatagcttggtgtaactctttctcttccctgacatttccttttctccagttgtctcttctgttcataatcacttctttttctcgttttctttcttttttccctccggctgcttttttttctgtccttggataccctgatgttttcctgtttgatttttctccatctgagttttttgtcctgcttccttctctcattcatcttctctcagttttctttgttttccttatcttagtcctttcttgcacatgttttgtctcttctttcactgtttcccttacttttcccagttctgtgcatgccttagttttttctcttcctacctttcttatttctaactccttttcctattatttcttatttctaactcctattcctacctttcttatttctaactccttttccttaaccttattttgataagctcagtactcttttaagtgagtctctggctcaggggcatgctttattttatgaattcccaactaaagagatgttccccctaaggggcaaaattgtttttttgaggagaggtgaaataatcttactttttttatgtgtaaatcacagatacacattcagtacattggtagaggcacagcctatctgtggtgttaatatttcatcggggagataattaggaaaacatgtctaagggggtgataataaaaaagattgagaaaaactgctatattctcaagagtgttattaacatgtgttctgtgatgggccttttctgtaggaagagacacctctgtttcttgctgcccgggaggggagctatgaagcagccaagatcctgttagaccattttgccaatcgagacatcacagaccatatggatcgtcttccccgggatgtggctcgggatcgcatgcaccatgacattgtgcgccttctggatgaatacaatgtgaccccaagccctccaggcaccgtgttgacttctgctctctcacctgtcatctgtgggcccaacagatctttcctcagcctgaagcacaccccaatgggcaagaagtctagacggcccagtgccaagagtaccatgcctactagcctccctaaccttgccaaggaggcaaaggatgccaagggtagtaggaggaagaagtctctgagtgagaaggtccaactgtctgagagttcagtaactttatcccctgttgattccctagaatctcctcacacgtatgtttccgacaccacatcctctccaatgattacatcccctgggatcttacaggcctcacccaaccctatgttggccactgccgcccctcctgccccagtccatgcccagcatgcactatctttttctaaccttcatgaaatgcagcctttggcacatggggccagcactgtgcttccctcagtgagccagttgctatcccaccaccacattgtgtctccaggcagtggcagtgctggaagcttgagtaggctccatccagtcccagtcccagcagattggatgaaccgcatggaggtgaatgagacccagtacaatgagatgtttggtatggtcctggctccagctgagggcacccatcctggcatagctccccagagcaggccacctgaagggaagcacataaccacccctcgggagcccttgccccccattgtgactttccagctcatccctaaaggcagtattgcccaaccagcgggggctccccagcctcagtccacctgccctccagctgttgcgggccccctgcccaccatgtaccagattccagaaatggcccgtttgcccagtgtggctttccccactgccatgatgccccagcaggacgggcaggtagctcagaccattctcccagcctatcatcctttcccagcctctgtgggcaagtaccccacacccccttcacagcacagttatgcttcctcaaatgctgctgagcgaacacccagtcacagtggtcacctccagggtgagcatccctacctgacaccatccccagagtctcctgaccagtggtcaagttcatcaccccactctgcttctgactggtcagatgtgaccaccagccctacccctgggggtgctggaggaggtcagcggggacctgggacacacatgtctgagccaccacacaacaacatgcaggtttatgcgtgagagagtccacctccagtgtagagacataactgacttttgtaaatgctgctgaggaacaaatgaaggtcatccgggagagaaatgaagaaatctctggagccagcttctagaggtaggaaagagaagatgttcttattcagataatgcaagagaagcaattcgtcagtttcactgggtatctgcaaggcttattgattattctaatctaataagacaagtttgtggaaatgcaagatgaatacaagccttgggtccatgtttactctcttctatttggagaataagatggatgcttattgaagcccagacattcttgcagcttggactgcattttaagccctgcaggcttctgccatatccatgagaagattctacactagcgtcctgttgggaattatgccctggaattctgcctgaattgacctacgcatctcctcctccttggacattcttttgtcttcatttggtgcttttggttttgcacctctccgtgattgtagccctaccagcatgttatagggcaagacctttgtgcttttgatcattctggcccatgaaagcaactttggtctcctttcccctcctgtcttcccggtatcccttggagtctcacaaggtttactttggtatggttctcagcacaaacctttcaagtatgttgtttctttggaaaatggacatactgtattgtgttctcctgcatatatcattcctggagagagaaggggagaagaatacttttcttcaacaaattttgggggcaggagatcccttcaagaggctgcaccttaatttttcttgtctgtgtgcaggtcttcatataaactttaccaggaagaagggtgtgagtttgttgtttttctgtgtatgggcctggtcagtgtaaagttttatccttgatagtctagttactatgaccctccccacttttttaaaaccagaaaaaggtttggaatgttggaatgaccaagagacaagttaactcgtgcaagagccagttacccacccacaggtccccctacttcctgccaagcattccattgactgcctgtatggaacacatttgtcccagatctgagcattctaggcctgtttcactcactcacccagcatatgaaactagtcttaactgttgagcctttcctttcatatccacagaagacactgtctcaaatgttgtacccttgccatttaggactgaactttccttagcccaagggacccagtgacagttgtcttccgtttgtcagatgatcagtctctactgattatcttgctgcttaaaggcctgctcaccaatctttctttcacaccgtgtggtccgtgttactggtatacccagtatgttctcactgaagacatggactttatatgttcaagtgcaggaattggaaagttggacttgttttctatgatccaaaacagccctataagaaggttggaaaaggaggaactatatagcagcctttgctattttctgctaccatttcttttcctctgaagcggccatgacattccctttggcaactaacgtagaaactcaacagaacattttcctttcctagagtcaccttttagatgataatggacaactatagacttgctcattgttcagactgattgcccctcacctgaatccactctctgtattcatgctcttggcaatttctttgactttcttttaagggcagaagcattttagttaattgtagataaagaatagttttcttcctcttctccttgggccagttaataattggtccatggctacactgcaacttccgtccagtgctgtgatgcccatgacacctgcaaaataagttctgcctgggcattttgtagatattaacaggtgaattcccgactcttttggtttgaatgacagttctcattccttctatggctgcaagtatgcatcagtgcttcccacttacctgatttgtctgtcggtggccccatatggaaaccctgcgtgtctgttggcataatagtttacaaatggttttttcagtcctatccaaatttattgaaccaacaaaaataattacttctgccctgagataagcagattaagtttgttcattctctgctttattctctccatgtggcaacattctgtcagcctctttcatagtgtgcaaacattttatcattctaaatggtgactctctgcccttggacccatttattattcacagatggggagaacctatctgcatggacctctgtggaccacagcgtacctgcccctttctgccctcctgctccagccccacttctgaaagtatcagctactgatccagccactggatattttatatcctcccttttccttaagcacaatgtcagaccaaattgcttgtttctttttcttggactactttaatttggatcctttgggtttggagaaagggaatgtgaaagctgtcattacagacaacaggtttcagtgatgaggaggacaacactgcctttcaaactttttactgatctcttagattttaagaactcttgaattgtgtggtatctaataaaagggaaggtaagatggataatcactttctcatttgggttctgaattggagactcagtttttatgagacacatcttttatgccatgtatagatcctcccctgctatttttggtttatttttattgttataaatgctttctttctttgactcctcttctgcctgcctttggggataggtttttttgtttgtttatttgcttcctctgttttgttttaagcatcattttcttatgtgaggtggggaagggaaaggtatgagggaaagagagtctgagaattaaaatattttagtataagcaattggctgtgatgctcaaatccattgcatcctcttattgaatttgccaatttgtaatttttgcataataaagaaccaaaggtgtaatgttttgttgagaggtggtttagggattttggccctaaccaatacattgaatgtatgatgactatttgggaggacacatttatgtacccagaggcccccactaataagtggtactatggttacttccttgtgtacatttctcttaaaagtgatattatatctgtttgtatgagaaacccagtaaccaataaaatgaccgcatattcctgactaaacgtagtaaggaaaatgcacactttgtttttacttttccgtttcattctaaaggtagttaagatgaaatttatatgaaagcatttttatcacaaaataaaaaaggtttgccaagctcagtggtgttgtattttttattttccaatactgcatccatggcctggcagtgttacctcatgatgtcataatttgctgagagagcaaattttcttttctttctgaatcccacaaagcctagcaccaaacttctttttttcttcctttaattagatcataaataaatgatcctggggaaaaagcatctgtcaaataggaaacatcacaaaactgagcactcttctgtgcactagccatagctggtgacaaacagatggttgctcagggacaaggtgccttccaatggaaatgcgaagtagttgctatagcaagaattgggaactgggatataagtcataatattaattatgctgttatgtaaatgattggtttgtaacattccttaagtgaaatttgtgtagaacttaatatacaggattataaaataatattttgtgtataaatttgttataagttcacattcatacatttatttataaagtcagtgagatatttgaacatgaatacttgatgttgttaatatgtatggcattagcagtctcatattgaccctagcatcttatttccattttccattattttgcagaaacttatcagtcacacatgcatagttataaatggtaacagttttttttttaatgacttgaaatcttaggatatgcttctgctaagcagataaaggagccttgttcaagatctgcacagttaaggttttaacgtaatttagcattaattggttatgttcagaatgttttaattttatataaaacattttagtcttcatattgggttttataatggtggaagaataaaatgaatttgagagctaaggaatgatcacctgtgcaggccggccactcagagctcaagttgccacttgcaaactatttgggaactaaattgctccatgaaaacctgcttcagtggatccaggttgcaggccattgggtggctgaccacatcctgcccctgcaattttaagtcttgaatgctagcctttgattatgcagggaggactcggggcactcccagacgtgaaagccaaaaaaggagagtagggccgaagccaagtgccaccccatcacaaagctggagcagtaggcttggacactagaccatgagcatgccaggagaggggctagtgcctcagtcatgcaactaaaaagcttcacgacccaaggagaggggctgcttcctttatcagaacaacctccttaatgcccacatgtttttcaggatgtgggcagtgggggaactgcgagcctgttaattgcctctgccactacgtctttcagccacagggtatatgaagttgtgtgtgtgttgtgtgtgtgaaagaatacaacttttccttcttttgccttagcacttagcaaaacacatttaaaatatttgtaccagtcaagtcccatccacaaagtgtgggcacactcaaattaggatacatcaaagagggctatttacaaaggattaattacaaaagtgtgtgtgcaatgtcagggaccgacacacagttgtgcagaaacccgtgttagcatcagcagagctatttccacttgtgggcccaaaaggccaggagaagtcactaggacctggaagcagagtcctgtagagcaggtccccaaccccccgggccacagactggataccttagcctgtggggaaccgttaggaaccgggctgcacagcaggaggtgagcagcaggcaagcaagcattactgcctgagctccacctcctgtcagatcagcagtggcgttagattctcataggagcatgaaccctattgtgaactgcatatgtgaaggatctaggttgcgcactccttatgataatctaatgtctgatgatctgagatggaagagtttcatccccaaaccattccctggccccagtccatggaaatattgtcttccacgaaactggttcctggtgccaaaaaggttggagactgctgctgtagagcaggctacctcaagaagagctgtgacttccttcaaagc

SEQ ID NO: 5 - DNA, 25 bp, Artificial Sequence
  PCR Primer
tggcaactaacgtagaaactcaaca

SEQ ID NO: 6 - DNA, 23 bp, Artificial Sequence
  PCR Primer
tgccaagagcatgaatacagaga

SEQ ID NO: 7 - DNA, 37 bp, Artificial Sequence
  PCR Probe
acaactatagacttgctcattgttcagactgattgcc

SEQ ID NO: 8 - DNA, 19 bp, Artificial Sequence
  PCR Primer
gaaggtgaaggtcggagtc

SEQ ID NO: 9 - DNA, 20 bp, Artificial Sequence
  PCR Primer
gaagatggtgatgggatttc

SEQ ID NO: 10 - DNA, 20 bp, Artificial Sequence
  PCR Probe
caagcttcccgttctcagcc

SEQ ID NO: 11 - DNA, 11189 bp, Homo sapiens
  CDS (13)...(7428)
gcgaccgagaagatgcccgccctgcgccccgctctgctgtgggcgctgctggcgctctggctgtgctgcgcgacccccgcgcatgcattgcagtgtcgagatggctatgaaccctgtgtaaatgaaggaatgtgtgttacctaccacaatggcacaggatactgcaaatgtccagaaggcttcttgggggaatattgtcaacatcgagacccctgtgagaagaaccgctgccagaatggtgggacttgtgtggcccaggccatgctggggaaagccacgtgccgatgtgcctcagggtttacaggagaggactgccagtactcgacatctcatccatgctttgtgtctcgaccctgcctgaatggcggcacatgccatatgctcagccgggatacctatgagtgcacctgtcaagtcgggtttacaggtaaggagtgccaatggaccgatgcctgcctgtctcatccctgtgcaaatggaagtacctgtaccactgtggccaaccagttctcctgcaaatgcctcacaggcttcacagggcagaaatgtgagactgatgtcaatgagtgtgacattccaggacactgccagcatggtggcacctgcctcaacctgcctggttcctaccagtgccagtgccttcagggcttcacaggccagtactgtgacagcctgtatgtgccctgtgcaccctcgccttgtgtcaatggaggcacctgtcggcagactggtgacttcacttttgagtgcaactgccttccaggttttgaagggagcacctgtgagaggaatattgatgactgccctaaccacaggtgtcagaatggaggggtttgtgtggatggggtcaacacttacaactgccgctgtcccccacaatggacaggacagttctgcacagaggatgtggatgaatgcctgctgcagcccaatgcctgtcaaaatgggggcacctgtgccaaccgcaatggaggctatggctgtgtatgtgtcaacggctggagtggagatgactgcagtgagaacattgatgattgtgccttcgcctcctgtactccaggctccacctgcatcgaccgtgtggcctccttctcttgcatgtgcccagaggggaaggcaggtctcctgtgtcatctggatgatgcatgcatcagcaatccttgccacaagggggcactgtgtgacaccaaccccctaaatgggcaatatatttgcacctgcccacaaggctacaaaggggctgactgcacagaagatgtggatgaatgtgccatggccaatagcaatccttgtgagcatgcaggaaaatgtgtgaacacggatggcgccttccactgtgagtgtctgaagggttatgcaggacctcgttgtgagatggacatcaatgagtgccattcagacccctgccagaatgatgctacctgtctggataagattggaggcttcacatgtctgtgcatgccaggtttcaaaggtgtgcattgtgaattagaaataaatgaatgtcagagcaacccttgtgtgaacaatgggcagtgtgtggataaagtcaatcgtttccagtgcctgtgtcctcctggtttcactgggccagtttgccagattgatattgatgactgttccagtactccgtgtctgaatggggcaaagtgtatcgatcacccgaatggctatgaatgccagtgtgccacaggtttcactggtgtgttgtgtgaggagaacattgacaactgtgaccccgatccttgccaccatggtcagtgtcaggatggtattgattcctacacctgcatctgcaatcccgggtacatgggcgccatctgcagtgaccagattgatgaatgttacagcagcccttgcctgaacgatggtcgctgcattgacctggtcaatggctaccagtgcaactgccagccaggcacgtcaggggttaattgtgaaattaattttgatgactgtgcaagtaacccttgtatccatggaatctgtatggatggcattaatcgctacagttgtgtctgctcaccaggattcacagggcagagatgtaacattgacattgatgagtgtgcctccaatccctgtcgcaagggtgcaacatgtatcaacggtgtgaatggtttccgctgtatatgccccgagggaccccatcaccccagctgctactcacaggtgaacgaatgcctgagcaatccctgcatccatggaaactgtactggaggtctcagtggatataagtgtctctgtgatgcaggctgggttggcatcaactgtgaagtggacaaaaatgaatgcctttcgaatccatgccagaatggaggaacttgtgacaatctggtgaatggatacaggtgtacttgcaagaagggctttaaaggctataactgccaggtgaatattgatgaatgtgcctcaaatccatgcctgaaccaaggaacctgctttgatgacataagtggctacacttgccactgtgtgctgccatacacaggcaagaattgtcagacagtattggctccctgttccccaaacccttgtgagaatgctgctgtttgcaaagagtcaccaaattttgagagttatacttgcttgtgtgctcctggctggcaaggtcagcggtgtaccattgacattgacgagtgtatctccaagccctgcatgaaccatggtctctgccataacacccagggcagctacatgtgtgaatgtccaccaggcttcagtggtatggactgtgaggaggacattgatgactgccttgccaatccttgccagaatggaggttcctgtatggatggagtgaatactttctcctgcctctgccttccgggtttcactggggataagtgccagacagacatgaatgagtgtctgagtgaaccctgtaagaatggagggacctgctctgactacgtcaacagttacacttgcaagtgccaggcaggatttgatggagtccattgtgagaacaacatcaatgagtgcactgagagctcctgtttcaatggtggcacatgtgttgatgggattaactccttctcttgcttgtgccctgtgggtttcactggatccttctgcctccatgagatcaatgaatgcagctctcatccatgcctgaatgagggaacgtgtgttgatggcctgggtacctaccgctgcagctgccccctgggctacactgggaaaaactgtcagaccctggtgaatctctgcagtcggtctccatgtaaaaacaaaggtacttgcgttcagaaaaaagcagagtcccagtgcctatgtccatctggatgggctggtgcctattgtgacgtgcccaatgtctcttgtgacatagcagcctccaggagaggtgtgcttgttgaacacttgtgccagcactcaggtgtctgcatcaatgctggcaacacgcattactgtcagtgccccctgggctatactgggagctactgtgaggagcaactcgatgagtgtgcgtccaacccctgccagcacggggcaacatgcagtgacttcattggtggatacagatgcgagtgtgtcccaggctatcagggtgtcaactgtgagtatgaagtggatgagtgccagaatcagccctgccagaatggaggcacctgtattgaccttgtgaaccatttcaagtgctcttgcccaccaggcactcggggcctactctgtgaagagaacattgatgactgtgcccggggtccccattgccttaatggtggtcagtgcatggataggattggaggctacagttgtcgctgcttgcctggctttgctggggagcgttgtgagggagacatcaacgagtgcctctccaacccctgcagctctgagggcagcctggactgtatacagctcaccaatgactacctgtgtgtttgccgtagtgcctttactggccggcactgtgaaaccttcgtcgatgtgtgtccccagatgccctgcctgaatggagggacttgtgctgtggccagtaacatgcctgatggtttcatttgccgttgtcccccgggattttccggggcaaggtgccagagcagctgtggacaagtgaaatgtaggaagggggagcagtgtgtgcacaccgcctctggaccccgctgcttctgccccagtccccgggactgcgagtcaggctgtgccagtagcccctgccagcacgggggcagctgccaccctcagcgccagcctccttattactcctgccagtgtgccccaccattctcgggtagccgctgtgaactctacacggcaccccccagcacccctcctgccacctgtctgagccagtattgtgccgacaaagctcgggatggcgtctgtgatgaggcctgcaacagccatgcctgccagtgggatgggggtgactgttctctcaccatggagaacccctgggccaactgctcctccccacttccctgctgggattatatcaacaaccagtgtgatgagctgtgcaacacggtcgagtgcctgtttgacaactttgaatgccaggggaacagcaagacatgcaagtatgacaaatactgtgcagaccacttcaaagacaaccactgtgaccaggggtgcaacagtgaggagtgtggttgggatgggctggactgtgctgctgaccaacctgagaacctggcagaaggtaccctggttattgtggtattgatgccacctgaacaactgctccaggatgctcgcagcttcttgcgggcactgggtaccctgctccacaccaacctgcgcattaagcgggactcccagggggaactcatggtgtacccctattatggtgagaagtcagctgctatgaagaaacagaggatgacacgcagatcccttcctggtgaacaagaacaggaggtggctggctctaaagtctttctggaaattgacaaccgccagtgtgttcaagactcagaccactgcttcaagaacacggatgcagcagcagctctcctggcctctcacgccatacaggggaccctgtcataccctcttgtgtctgtcgtcagtgaatccctgactccagaacgcactcagctcctctatctccttgctgttgctgttgtcatcattctgtttattattctgctgggggtaatcatggcaaaacgaaagcgtaagcatggctctctctggctgcctgaaggtttcactcttcgccgagatgcaagcaatcacaagcgtcgtgagccagtgggacaggatgctgtggggctgaaaaatctctcagtgcaagtctcagaagctaacctaattggtactggaacaagtgaacactgggtcgatgatgaagggccccagccaaagaaagtaaaggctgaagatgaggccttactctcagaagaagatgaccccattgatcgacggccatggacacagcagcaccttgaagctgcagacatccgtaggacaccatcgctggctctcacccctcctcaggcagagcaggaggtggatgtgttagatgtgaatgtccgtggcccagatggctgcaccccattgatgttggcttctctccgaggaggcagctcagatttgagtgatgaagatgaagatgcagaggactcttctgctaacatcatcacagacttggtctaccagggtgccagcctccaggcccagacagaccggactggtgagatggccctgcaccttgcagcccgctactcacgggctgatgctgccaagcgtctcctggatgcaggtgcagatgccaatgcccaggacaacatgggccgctgtccactccatgctgcagtggcagctgatgcccaaggtgtcttccagattctgattcgcaaccgagtaactgatctagatgccaggatgaatgatggtactacacccctgatcctggctgcccgcctggctgtggagggaatggtggcagaactgatcaactgccaagcggatgtgaatgcagtggatgaccatggaaaatctgctcttcactgggcagctgctgtcaataatgtggaggcaactcttttgttgttgaaaaatggggccaaccgagacatgcaggacaacaaggaagagacacctctgtttcttgctgcccgggaggggagctatgaagcagccaagatcctgttagaccattttgccaatcgagacatcacagaccatatggatcgtcttccccgggatgtggctcgggatcacatgcaccatgacattgtgcgccttctggatgaatacaatgtgaccccaagccctccaggcaccgtgttgacttctgctctctcacctgtcatctgtgggcccaacagatctttcctcagcctgaagcacaccccaatgggcaagaagtctagacggcccagtgccaagagtaccatgcctactagcctccctaaccttgccaaggaggcaaaggatgccaagggtagtaggaggaagaagtctctgagtgagaaggtccaactgtctgagagttcagtaactttatcccctgttgattccctagaatctcctcacacgtatgtttccgacaccacatcctctccaatgattacatcccctgggatcttacaggcctcacccaaccctatgttggccactgccgcccctcctgccccagtccatgcccagcatgcactatctttttctaaccttcatgaaatgcagcctttggcacatggggccagcactgtgcttccctcagtgagccagttgctatcccaccaccacattgtgtctccaggcagtggcagtgctggaagcttgagtaggctccatccagtcccagtcccagcagattggatgaaccgcatggaggtgaatgagacccagtacaatgagatgtttggtatggtcctggctccagctgagggcacccatcctggcatagctccccagagcaggccacctgaagggaagcacataaccacccctcgggagcccttgccccccattgtgactttccagctcatccctaaaggcagtattgcccaaccagcgggggctccccagcctcagtccacctgccctccagctgttgcgggccccctgcccaccatgtaccagattccagaaatggcccgtttgcccagtgtggctttccccactgccatgatgccccagcaggacgggcaggtagctcagaccattctcccagcctatcatcctttcccagcctctgtgggcaagtaccccacacccccttcacagcacagttatgcttcctcaaatgctgctgagcgaacacccagtcacagtggtcacctccagggtgagcatccctacctgacaccatccccagagtctcctgaccagtggtcaagttcatcaccccactctgcttctgactggtcagatgtgaccaccagccctacccctgggggtgctggaggaggtcagcggggacctgggacacacatgtctgagccaccacacaacaacatgcaggtttatgcgtgagagagtccacctccagtgtagagacataactgacttttgtaaatgctgctgaggaacaaatgaaggtcatccgggagagaaatgaagaaatctctggagccagcttctagaggtaggaaagagaagatgttcttattcagataatgcaagagaagcaattcgtcagtttcactgggtatctgcaaggcttattgattattctaatctaataagacaagtttgtggaaatgcaagatgaatacaagccttgggtccatgtttactctcttctatttggagaataagatggatgcttattgaagcccagacattcttgcagcttggactgcattttaagccctgcaggcttctgccatatccatgagaagattctacactagcgtcctgttgggaattatgccctggaattctgcctgaattgacctacgcatctcctcctccttggacattcttttgtcttcatttggtgcttttggttttgcacctctccgtgattgtagccctaccagcatgttatagggcaagacctttgtgcttttgatcattctggcccatgaaagcaactttggtctcctttcccctcctgtcttcccggtatcccttggagtctcacaaggtttactttggtatggttctcagcacaaacctttcaagtatgttgtttctttggaaaatggacatactgtattgtgttctcctgcatatatcattcctggagagagaaggggagaagaatacttttcttcaacaaattttgggggcaggagatcccttcaagaggctgcaccttaatttttcttgtctgtgtgcaggtcttcatataaactttaccaggaagaagggtgtgagtttgttgtttttctgtgtatgggcctggtcagtgtaaagttttatccttgatagtctagttactatgaccctccccacttttttaaaaccagaaaaaggtttggaatgttggaatgaccaagagacaagttaactcgtgcaagagccagttacccacccacaggtccccctacttcctgccaagcattccattgactgcctgtatggaacacatttgtcccagatctgagcattctaggcctgtttcactcactcacccagcatatgaaactagtcttaactgttgagcctttcctttcatatccacagaagacactgtctcaaatgttgtacccttgccatttaggactgaactttccttagcccaagggacccagtgacagttgtcttccgtttgtcagatgatcagtctctactgattatcttgctgcttaaaggcctgctcaccaatctttctttcacaccgtgtggtccgtgttactggtatacccagtatgttctcactgaagacatggactttatatgttcaagtgcaggaattggaaagttggacttgttttctatgatccaaaacagccctataagaaggttggaaaaggaggaactatatagcagcctttgctattttctgctaccatttcttttcctctgaagcggccatgacattccctttggcaactaacgtagaaactcaacagaacattttcctttcctagagtcaccttttagatgataatggacaactatagacttgctcattgttcagactgattgcccctcacctgaatccactctctgtattcatgctcttggcaatttctttgactttcttttaagggcagaagcattttagttaattgtagataaagaatagttttcttcctcttctccttgggccagttaataattggtccatggctacactgcaacttccgtccagtgctgtgatgcccatgacacctgcaaaataagttctgcctgggcattttgtagatattaacaggtgaattcccgactcttttggtttgaatgacagttctcattccttctatggctgcaagtatgcatcagtgcttcccacttacctgatttgtctgtcggtggccccatatggaaaccctgcgtgtctgttggcataatagtttacaaatggttttttcagtcctatccaaatttattgaaccaacaaaaataattacttctgccctgagataagcagattaagtttgttcattctctgctttattctctccatgtggcaacattctgtcagcctctttcatagtgtgcaaacattttatcattctaaatggtgactctctgcccttggacccatttattattcacagatggggagaacctatctgcatggacctctgtggaccacagcgtacctgcccctttctgccctcctgctccagccccacttctgaaagtatcagctactgatccagccactggatattttatatcctcccttttccttaagcacaatgtcagaccaaattgcttgtttctttttcttggactactttaatttggatcctttgggtttggagaaagggaatgtgaaagctgtcattacagacaacaggtttcagtgatgaggaggacaacactgcctttcaaactttttactgatctcttagattttaagaactcttgaattgtgtggtatctaataaaagggaaggtaagatggataatcactttctcatttgggttctgaattggagactcagtttttatgagacacatcttttatgccatgtatagatcctcccctgctatttttggtttatttttattgttataaatgctttctttctttgactcctcttctgcctgcctttggggataggtttttttgtttgtttatttgcttcctctgttttgttttaagcatcattttcttatgtgaggtggggaagggaaaggtatgagggaaagagagtctgagaattaaaatattttagtataagcaattggctgtgatgctcaaatccattgcatcctcttattgaatttgccaatttgtaatttttgcataataaagaaccaaaggtgtaatgttttgttgagaggtggtttagggattttggccctaaccaatacattgaatgtatgatgactatttgggaggacacatttatgtacccagaggcccccactaataagtggtactatggttacttccttgtgtacatttctcttaaaagtgatattatatctgtttgtatgagaaacccagtaaccaataaaatgaccgcatattcctgactaaacgtagtaaggaaaatgcacactttgtttttacttttccgtttcattctaaaggtagttaagatgaaatttatatgaaagcatttttatcacaaaataaaaaaggtttgccaagctcagtggtgttgtattttttattttccaatactgcatccatggcctggcagtgttacctcatgatgtcataatttgctgagagagcaaattttcttttctttctgaatcccacaaagcctagcaccaaacttctttttttcttcctttaattagatcataaataaatgatcctggggaaaaagcatctgtcaaataggaaacatcacaaaactgagcactcttctgtgcactagccatagctggtgacaaacagatggttgctcagggacaaggtgccttccaatggaaatgcgaagtagttgctatagcaagaattgggaactgggatataagtcataatattaattatgctgttatgtaaatgattggtttgtaacattccttaagtgaaatttgtgtagaacttaatatacaggattataaaataatattttgtgtataaatttgttataagttcacattcatacatttatttataaagtcagtgagatatttgaacatgaaaaaaaaaa

SEQ ID NO: 12 - DNA, 1161 bp, H. sapiens
ggcacgagggctgcacacccgagaaagtttcagccaaacttcgggcggcggctgaggcggcggccgaggagcggcggactcggggcgcggggagtcgaggcatttgcgctgggcttcggagcgtagcgccagggcctgagcctttgaagcaggaggaggggaggagagagtggggctcctctatcgggaccccctccccatgtggatctgcccaggcggcggcggcggcggcggcggaggaggcgaccgagaagatacccgccctgcgccccgctctgctgtgggcgctgctggcgctctggctgtgctgcgcgacccccgcgcatgcattgcagtgtcgagatggctatgaaccctgtgtaaatgaaggaatgtgtgttacctaccacaatggcacaggatactgcaaatgtccagaaggcttcttgggggaatattgtcaacatcgagacccctgtgagaagaaccgctgccagaatggtgggacttgtgtggcccaggccatgctggggaaagccacgtgccgatgtgcctcagggtttacaggagaggactgccagtactcgacatctcatccatgctttgtgtctcgaccttgcctgaatggcggcacatgccatatgctcagccgggatacctatgagtgcacctgtcaagtcgggtttacaggtaaggagtgccaatggaccgatgcctgcctgtctcatccctgtgcaaatggaagtacctgtaccactgtggccaaccagttctcctgcaaatgcctcacaggcttcacagggcagaagtgtgagactgatgtcaatgagtgtgacattccaggacactgccagcatggtggcacctgcctcaacctgcctggttcctaccagtgccagtgccttcagggcttcacaggccagtactgtgacagcctgtatgtgccctgtgcaccctcgccttgtgtcaatggaggcacctgtcggcagactggtgacttcacttttgagtgcaactgccttccagaaacagtgagaagaggaacagagctctgggaaagagacagggaagtctggaatggaaaagaacacgatgagaattagacactggaaaatatgtatgtgtggttaataaagtgctttaaactgaattgaaaaaaaaaaaaaaaaaaaaaaaaaaaaa

SEQ ID NO: 13 - DNA, 598 bp, H. sapiens
agcggggcgcttcttcctgcgggaaacccctgggtgcccaaggcggcggggccgaggccgcggcgacagtggggcggggcttgcggtgggaggaggcggctgaggcggaaggacacacgaggctgcttcgctgcacacccgagaaagtttcagccaaacttcgggcggcggctgaggcggcggccgaggagcggcggactcggggcgcggggagtcgaggcatttgcgctgggcttcggagcgtagcgccaggcctgagcctttgaagcaggaggaggggaggagagagtggggctcctctatcgggaccccctccccatgtggatctggcccaggcggcggcggcggcggcggcggaggaggcgaccgagaagatacccgccctgcgccccgctctgctgtgggcgctgctggcgctctggctgtgctgcgcgaccccgcgcatgcattgcagtgtcgagatggctatgaaccctgtgtacatgaaggactgtgtgttacctaccacaatggcacaggatactgcaaatgtccagaaggcttctggggggaatatgtcaacatcgagacccctgttgagaagaaccgtgccagaatg

SEQ ID NO: 14 - DNA, 20 bp, Artificial Sequence
  Antisense Oligonucleotide
tctcgatgttgacaatattc

SEQ ID NO: 15 - DNA, 20 bp, Artificial Sequence
  Antisense Oligonucleotide
cactccttacctgtaaaccc

SEQ ID NO: 16 - DNA, 20 bp, Artificial Sequence
  Antisense Oligonucleotide
ggcatttgcaggagaactgg

SEQ ID NO: 17 - DNA, 20 bp, Artificial Sequence
  Antisense Oligonucleotide
gtgctcccttcaaaacctgg

SEQ ID NO: 18 - DNA, 20 bp, Artificial Sequence
  Antisense Oligonucleotide
tttcctgcatgctcacaagg

SEQ ID NO: 19 - DNA, 20 bp, Artificial Sequence
  Antisense Oligonucleotide
ccttcagacactcacagtgg

SEQ ID NO: 20 - DNA, 20 bp, Artificial Sequence
  Antisense Oligonucleotide
ctttgaaacctggcatgcac

SEQ ID NO: 21 - DNA, 20 bp, Artificial Sequence
  Antisense Oligonucleotide
caatgcacacctttgaaacc

SEQ ID NO: 22 - DNA, 20 bp, Artificial Sequence
  Antisense Oligonucleotide
tagccattcgggtgatcgat

SEQ ID NO: 23 - DNA, 20 bp, Artificial Sequence
  Antisense Oligonucleotide
attcatagccattcgggtga

SEQ ID NO: 24 - DNA, 20 bp, Artificial Sequence
  Antisense Oligonucleotide
ctggcattcatagccattcg

SEQ ID NO: 25 - DNA, 20 bp, Artificial Sequence
  Antisense Oligonucleotide
ggcacactggcattcatagc

SEQ ID NO: 26 - DNA, 20 bp, Artificial Sequence
  Antisense Oligonucleotide
cctgtggcacactggcattc

SEQ ID NO: 27 - DNA, 20 bp, Artificial Sequence
  Antisense Oligonucleotide
ttgcagatgcaggtgtagga

SEQ ID NO: 28 - DNA, 20 bp, Artificial Sequence
  Antisense Oligonucleotide
cattcatcaatctggtcact

SEQ ID NO: 29 - DNA, 20 bp, Artificial Sequence
  Antisense Oligonucleotide
gtcatcaaaattaatttcac

SEQ ID NO: 30 - DNA, 20 bp, Artificial Sequence
  Antisense Oligonucleotide
gcacagtcatcaaaattaat

SEQ ID NO: 31 - DNA, 20 bp, Artificial Sequence
  Antisense Oligonucleotide
tggaggcacactcatcaatg

SEQ ID NO: 32 - DNA, 20 bp, Artificial Sequence
  Antisense Oligonucleotide
ttcacctgtgagtagcagct

SEQ ID NO: 33 - DNA, 20 bp, Artificial Sequence
  Antisense Oligonucleotide
agacctccagtacagtttcc

SEQ ID NO: 34 - DNA, 20 bp, Artificial Sequence
  Antisense Oligonucleotide
cactgagacctccagtacag

SEQ ID NO: 35 - DNA, 20 bp, Artificial Sequence
  Antisense Oligonucleotide
aagcaggttccttggttcag

SEQ ID NO: 36 - DNA, 20 bp, Artificial Sequence
  Antisense Oligonucleotide
tgccagccaggagcacacaa

SEQ ID NO: 37 - DNA, 20 bp, Artificial Sequence
  Antisense Oligonucleotide
tggcaaggattggcaaggca

SEQ ID NO: 38 - DNA, 20 bp, Artificial Sequence
  Antisense Oligonucleotide
ttcatgtctgtctggcactt

SEQ ID NO: 39 - DNA, 20 bp, Artificial Sequence
  Antisense Oligonucleotide
agggtctgacagtttttccc

SEQ ID NO: 40 - DNA, 20 bp, Artificial Sequence
  Antisense Oligonucleotide
tcaccagggtctgacagttt

SEQ ID NO: 41 - DNA, 20 bp, Artificial Sequence
  Antisense Oligonucleotide
gctggcacaagtgttcaaca

SEQ ID NO: 42 - DNA, 20 bp, Artificial Sequence
  Antisense Oligonucleotide
tcacagttgacaccctgata

SEQ ID NO: 43 - DNA, 20 bp, Artificial Sequence
  Antisense Oligonucleotide
catactcacagttgacaccc

SEQ ID NO: 44 - DNA, 20 bp, Artificial Sequence
  Antisense Oligonucleotide
cacttcatactcacagttga

SEQ ID NO: 45 - DNA, 20 bp, Artificial Sequence
  Antisense Oligonucleotide
tccccagcaaagccaggcaa

SEQ ID NO: 46 - DNA, 20 bp, Artificial Sequence
  Antisense Oligonucleotide
acttgtccacagctgctctg

SEQ ID NO: 47 - DNA, 20 bp, Artificial Sequence
  Antisense Oligonucleotide
ccatcccactggcaggcatg

SEQ ID NO: 48 - DNA, 20 bp, Artificial Sequence
  Antisense Oligonucleotide
tcatcacactggttgttgat

SEQ ID NO: 49 - DNA, 20 bp, Artificial Sequence
  Antisense Oligonucleotide
aagttgtcaaacaggcactc

SEQ ID NO: 50 - DNA, 20 bp, Artificial Sequence
  Antisense Oligonucleotide
cagtatttgtcatacttgca

SEQ ID NO: 51 - DNA, 20 bp, Artificial Sequence
  Antisense Oligonucleotide
ctgcacagtatttgtcatac

SEQ ID NO: 52 - DNA, 20 bp, Artificial Sequence
  Antisense Oligonucleotide
gtggtctgcacagtatttgt

SEQ ID NO: 53 - DNA, 20 bp, Artificial Sequence
  Antisense Oligonucleotide
ttgaagtggtctgcacagta

SEQ ID NO: 54 - DNA, 20 bp, Artificial Sequence
  Antisense Oligonucleotide
tgtctttgaagtggtctgca

SEQ ID NO: 55 - DNA, 20 bp, Artificial Sequence
  Antisense Oligonucleotide
tcacagtggttgtctttgaa

SEQ ID NO: 56 - DNA, 20 bp, Artificial Sequence
  Antisense Oligonucleotide
gactttagagccagccacct

SEQ ID NO: 57 - DNA, 20 bp, Artificial Sequence
  Antisense Oligonucleotide
agcttcaaggtgctgctgtg

SEQ ID NO: 58 - DNA, 20 bp, Artificial Sequence
  Antisense Oligonucleotide
tctgcagcttcaaggtgctg

SEQ ID NO: 59 - DNA, 20 bp, Artificial Sequence
  Antisense Oligonucleotide
gcctcctcggagagaagcca

SEQ ID NO: 60 - DNA, 20 bp, Artificial Sequence
  Antisense Oligonucleotide
gagctgcctcctcggagaga

SEQ ID NO: 61 - DNA, 20 bp, Artificial Sequence
  Antisense Oligonucleotide
accaagtctgtgatgatgtt

SEQ ID NO: 62 - DNA, 20 bp, Artificial Sequence
  Antisense Oligonucleotide
ggtagaccaagtctgtgatg

SEQ ID NO: 63 - DNA, 20 bp, Artificial Sequence
  Antisense Oligonucleotide
gcaaggtgcagggccatctc

SEQ ID NO: 64 - DNA, 20 bp, Artificial Sequence
  Antisense Oligonucleotide
tttccatggtcatccactgc

SEQ ID NO: 65 - DNA, 20 bp, Artificial Sequence
  Antisense Oligonucleotide
cagattttccatggtcatcc

SEQ ID NO: 66 - DNA, 20 bp, Artificial Sequence
  Antisense Oligonucleotide
acattattgacagcagctgc

SEQ ID NO: 67 - DNA, 20 bp, Artificial Sequence
  Antisense Oligonucleotide
gcctccttggcaaggttagg

SEQ ID NO: 68 - DNA, 20 bp, Artificial Sequence
  Antisense Oligonucleotide
aagccacactgggcaaacgg

SEQ ID NO: 69 - DNA, 20 bp, Artificial Sequence
  Antisense Oligonucleotide
atggtctgagctacctgccc

SEQ ID NO: 70 - DNA, 20 bp, Artificial Sequence
  Antisense Oligonucleotide
cccacagaggctgggaaagg

SEQ ID NO: 71 - DNA, 20 bp, Artificial Sequence
  Antisense Oligonucleotide
acttgcccacagaggctggg

SEQ ID NO: 72 - DNA, 20 bp, Artificial Sequence
  Antisense Oligonucleotide
gtcacatctgaccagtcaga

SEQ ID NO: 73 - DNA, 20 bp, Artificial Sequence
  Antisense Oligonucleotide
tggtggtcacatctgaccag

SEQ ID NO: 74 - DNA, 20 bp, Artificial Sequence
  Antisense Oligonucleotide
ggactctctcacgcataaac

SEQ ID NO: 75 - DNA, 20 bp, Artificial Sequence
  Antisense Oligonucleotide
aaaggtcttgccctataaca

SEQ ID NO: 76 - DNA, 20 bp, Artificial Sequence
  Antisense Oligonucleotide
cattccaaacctttttctgg

SEQ ID NO: 77 - DNA, 20 bp, Artificial Sequence
  Antisense Oligonucleotide
cagacaaatcaggtaagtgg

SEQ ID NO: 78 - DNA, 20 bp, Artificial Sequence
  Antisense Oligonucleotide
caaaacattacacctttggt

SEQ ID NO: 79 - DNA, 20 bp, Artificial Sequence
  Antisense Oligonucleotide
atttcacttaaggaatgtta

SEQ ID NO: 80 - DNA, 20 bp, Artificial Sequence
  Antisense Oligonucleotide
tcaggagatcgagaccatcc

SEQ ID NO: 81 - DNA, 20 bp, Artificial Sequence
  Antisense Oligonucleotide
agctccttacctggaaggca

SEQ ID NO: 82 - DNA, 20 bp, Artificial Sequence
  Antisense Oligonucleotide
ccaaccactacgggtcttgg

SEQ ID NO: 83 - DNA, 20 bp, Artificial Sequence
  Antisense Oligonucleotide
ccagtgaaacctttggaaag

SEQ ID NO: 84 - DNA, 20 bp, Artificial Sequence
  Antisense Oligonucleotide
atgttcttaccttgccagcc

SEQ ID NO: 85 - DNA, 20 bp, Artificial Sequence
  Antisense Oligonucleotide
ggcatactcactggcaaggc

SEQ ID NO: 86 - DNA, 20 bp, Artificial Sequence
  Antisense Oligonucleotide
aagcccttacttgcatgtct

SEQ ID NO: 87 - DNA, 20 bp, Artificial Sequence
  Antisense Oligonucleotide
gaatgacagagcaactgaag

SEQ ID NO: 88 - DNA, 20 bp, Artificial Sequence
  Antisense Oligonucleotide
ctttctcgggtgtgcagccc

SEQ ID NO: 89 - DNA, 20 bp, Artificial Sequence
  Antisense Oligonucleotide
gtatcttctcggtcgcctcc

SEQ ID NO: 90 - DNA, 20 bp, Artificial Sequence
  Antisense Oligonucleotide
cctgtctctttcccagagct

SEQ ID NO: 91 - DNA, 20 bp, Artificial Sequence
  Antisense Oligonucleotide
cgccgccttgggcacccagg

SEQ ID NO: 92 - DNA, 20 bp, H. sapiens
gaatattgtcaacatcgaga

SEQ ID NO: 93 - DNA, 20 bp, H. sapiens
ccagttctcctgcaaatgcc

SEQ ID NO: 94 - DNA, 20 bp, H. sapiens
ccaggttttgaagggagcac

SEQ ID NO: 95 - DNA, 20 bp, H. sapiens
ccttgtgagcatgcaggaaa

SEQ ID NO: 96 - DNA, 20 bp, H. sapiens
ccactgtgagtgtctgaagg

SEQ ID NO: 97 - DNA, 20 bp, H. sapiens
gtgcatgccaggtttcaaag

SEQ ID NO: 98 - DNA, 20 bp, H. sapiens
atcgatcacccgaatggcta

SEQ ID NO: 99 - DNA, 20 bp, H. sapiens
cgaatggctatgaatgccag

SEQ ID NO: 100 - DNA, 20 bp, H. sapiens
gaatgccagtgtgccacagg

SEQ ID NO: 101 - DNA, 20 bp, H. sapiens
tcctacacctgcatctgcaa

SEQ ID NO: 102 - DNA, 20 bp, H. sapiens
gtgaaattaattttgatgac

SEQ ID NO: 103 - DNA, 20 bp, H. sapiens
attaattttgatgactgtgc

SEQ ID NO: 104 - DNA, 20 bp, H. sapiens
cattgatgagtgtgcctcca

SEQ ID NO: 105 - DNA, 20 bp, H. sapiens
agctgctactcacaggtgaa

SEQ ID NO: 106 - DNA, 20 bp, H. sapiens
ggaaactgtactggaggtct

SEQ ID NO: 107 - DNA, 20 bp, H. sapiens
ctgtactggaggtctcagtg

SEQ ID NO: 108 - DNA, 20 bp, H. sapiens
ctgaaccaaggaacctgctt

SEQ ID NO: 109 - DNA, 20 bp, H. sapiens
ttgtgtgctcctggctggca

SEQ ID NO: 110 - DNA, 20 bp, H. sapiens
tgccttgccaatccttgcca

SEQ ID NO: 111 - DNA, 20 bp, H. sapiens
aagtgccagacagacatgaa

SEQ ID NO: 112 - DNA, 20 bp, H. sapiens
gggaaaaactgtcagaccct

SEQ ID NO: 113 - DNA, 20 bp, H. sapiens
aaactgtcagaccctggtga

SEQ ID NO: 114 - DNA, 20 bp, H. sapiens
tgttgaacacttgtgccagc

SEQ ID NO: 115 - DNA, 20 bp, H. sapiens
tatcagggtgtcaactgtga

SEQ ID NO: 116 - DNA, 20 bp, H. sapiens
tcaactgtgagtatgaagtg

SEQ ID NO: 117 - DNA, 20 bp, H. sapiens
ttgcctggctttgctgggga

SEQ ID NO: 118 - DNA, 20 bp, H. sapiens
cagagcagctgtggacaagt

SEQ ID NO: 119 - DNA, 20 bp, H. sapiens
catgcctgccagtgggatgg

SEQ ID NO: 120 - DNA, 20 bp, H. sapiens
atcaacaaccagtgtgatga

SEQ ID NO: 121 - DNA, 20 bp, H. sapiens
gagtgcctgtttgacaactt

SEQ ID NO: 122 - DNA, 20 bp, H. sapiens
tgcaagtatgacaaatactg

SEQ ID NO: 123 - DNA, 20 bp, H. sapiens
gtatgacaaatactgtgcag

SEQ ID NO: 124 - DNA, 20 bp, H. sapiens
acaaatactgtgcagaccac

SEQ ID NO: 125 - DNA, 20 bp, H. sapiens
tactgtgcagaccacttcaa

SEQ ID NO: 126 - DNA, 20 bp, H. sapiens
tgcagaccacttcaaagaca

SEQ ID NO: 127 - DNA, 20 bp, H. sapiens
ttcaaagacaaccactgtga

SEQ ID NO: 128 - DNA, 20 bp, H. sapiens
cacagcagcaccttgaagct

SEQ ID NO: 129 - DNA, 20 bp, H. sapiens
cagcaccttgaagctgcaga

SEQ ID NO: 130 - DNA, 20 bp, H. sapiens
tggcttctctccgaggaggc

SEQ ID NO: 131 - DNA, 20 bp, H. sapiens
aacatcatcacagacttggt

SEQ ID NO: 132 - DNA, 20 bp, H. sapiens
gcagtggatgaccatggaaa

SEQ ID NO: 133 - DNA, 20 bp, H. sapiens
ggatgaccatggaaaatctg

SEQ ID NO: 134 - DNA, 20 bp, H. sapiens
gcagctgctgtcaataatgt

SEQ ID NO: 135 - DNA, 20 bp, H. sapiens
cctaaccttgccaaggaggc

SEQ ID NO: 136 - DNA, 20 bp, H. sapiens
ccgtttgcccagtgtggctt

SEQ ID NO: 137 - DNA, 20 bp, H. sapiens
gggcaggtagctcagaccat

SEQ ID NO: 138 - DNA, 20 bp, H. sapiens
cctttcccagcctctgtggg

SEQ ID NO: 139 - DNA, 20 bp, H. sapiens
cccagcctctgtgggcaagt

SEQ ID NO: 140 - DNA, 20 bp, H. sapiens
tctgactggtcagatgtgac

SEQ ID NO: 141 - DNA, 20 bp, H. sapiens
ctggtcagatgtgaccacca

SEQ ID NO: 142 - DNA, 20 bp, H. sapiens
gtttatgcgtgagagagtcc

SEQ ID NO: 143 - DNA, 20 bp, H. sapiens
ccagaaaaaggtttggaatg

SEQ ID NO: 144 - DNA, 20 bp, H. sapiens
ccacttacctgatttgtctg

SEQ ID NO: 145 - DNA, 20 bp, H. sapiens
accaaaggtgtaatgttttg

SEQ ID NO: 146 - DNA, 20 bp, H. sapiens
tgccttccaggtaaggagct

SEQ ID NO: 147 - DNA, 20 bp, H. sapiens
ccaagacccgtagtggttgg

SEQ ID NO: 148 - DNA, 20 bp, H. sapiens
ctttccaaaggtttcactgg

SEQ ID NO: 149 - DNA, 20 bp, H. sapiens
gccttgccagtgagtatgcc

SEQ ID NO: 150 - DNA, 20 bp, H. sapiens
agacatgcaagtaagggctt

SEQ ID NO: 151 - DNA, 20 bp, H. sapiens
cttcagttgctctgtcattc

SEQ ID NO: 152 - DNA, 20 bp, H. sapiens
ggaggcgaccgagaagatac

(19) United States
(12) Patent Application Publication
(10) Pub. No.: US 2005/0208532 A1
(43) Pub. Date: Sep. 22, 2005

(54) Compositions and their uses directed to signal transducers

(76) Inventors: Susan M. Freier, San Diego, CA (US); Kenneth W. Dobie, Del Mar, CA (US)

Correspondence Address: FENWICK & WEST LLP, 801 CALIFORNIA STREET, MOUNTAIN VIEW, CA, 94014, US

(21) Appl. No.: 11/004,765

(22) Filed: Dec. 3, 2004

Related U.S. Application Data
(63) Continuation of application No. 10/983,197, filed on Nov. 4, 2004.
(63) Continuation-in-part of application No. 10/292,337, filed on Nov. 13, 2002.
(63) Continuation-in-part of application No. 10/303,165, filed on Nov. 22, 2002.
(63) Continuation-in-part of application No. 10/174,771, filed on Jun. 17, 2002, now abandoned.
(63) Continuation-in-part of application No. 10/210,290, filed on Jul. 31, 2002, now abandoned.
(63) Continuation-in-part of application No. 10/210,479, filed on Jul. 31, 2002.
(63) Continuation-in-part of application No. 10/211,908, filed on Jul. 31, 2002.
(63) Continuation-in-part of application No. 10/316,516, filed on Dec. 10, 2002, now abandoned.
(63) Continuation-in-part of application No. 10/317,270, filed on Dec. 10, 2002.
(63) Continuation-in-part of application No. 10/173,902, filed on Jun. 17, 2002.
(63) Continuation-in-part of application No. 10/174,456, filed on Jun. 17, 2002, now abandoned.
(63) Continuation-in-part of application No. 10/178,258, filed on Jun. 20, 2002, now abandoned.
(63) Continuation-in-part of application No. 10/187,659, filed on Jul. 1, 2002.
(63) Continuation-in-part of application No. 10/211,859, filed on Jul. 31, 2002.
(63) Continuation-in-part of application No. 10/210,429, filed on Jul. 31, 2002.
(63) Continuation-in-part of application No. 10/293,998, filed on Nov. 11, 2002.
(63) Continuation-in-part of application No. 10/316,242, filed on Dec. 9, 2002.
(63) Continuation-in-part of application No. 10/315,474, filed on Dec. 10, 2002.
(63) Continuation-in-part of application No. 10/317,803, filed on Dec. 11, 2002.
(63) Continuation-in-part of application No. 10/346,268, filed on Jan. 15, 2003.
(63) Continuation-in-part of application No. 10/299,089, filed on Nov. 16, 2002.
(63) Continuation-in-part of application No. 10/304,082, filed on Nov. 22, 2002.
(63) Continuation-in-part of application No. 10/304,098, filed on Nov. 22, 2002.
(63) Continuation-in-part of application No. 10/303,420, filed on Nov. 23, 2002.
(63) Continuation-in-part of application No. 10/300,236, filed on Nov. 19, 2002.
(63) Continuation-in-part of application No. 10/300,263, filed on Nov. 19, 2002, now abandoned.
(63) Continuation-in-part of application No. 10/302,571, filed on Nov. 22, 2002.
(63) Continuation-in-part of application No. 10/317,478, filed on Dec. 11, 2002, now abandoned.
(63) Continuation-in-part of application No. 10/301,832, filed on Nov. 21, 2002.
(63) Continuation-in-part of application No. 10/174,128, filed on Jun. 17, 2002, now abandoned.

(51) Int. Cl. C12Q 1/68; C07H 21/04

(52) U.S. Cl. 435/6; 536/23.1

(57) ABSTRACT

Compounds, compositions and methods are provided for modulating the expression of Notch2. The compositions comprise oligonucleotides, targeted to nucleic acid encoding Notch2. Methods of using these compounds for modulation of Notch2 expression and for diagnosis and treatment of diseases and conditions associated with expression of Notch2 are provided.

### RELATED APPLICATIONS

This application is continuation of co-pending U.S. patent application Ser. No. 10/983,197, filed Nov. 4, 2004 (Attorney Docket No. BNDL-0008US.P1 (23546-09781US), which is a continuation-in-part of the following U.S. patent applications: Ser. No. 10/292,337, filed Nov. 13, 2002; Ser. No. 10/303,165, filed Nov. 22, 2002; Ser. No. 10/174,771, filed Jun. 17, 2002; Ser. No. 10/210,290, filed Jul. 31, 2002; Ser. No. 10/210,479, filed Jul. 31, 2002; Ser. No. 10/211,908, filed Jul. 31, 2002; Ser. No. 10/174,128, filed Jun. 17, 2002; Ser. No. 10/316,516, filed Dec. 10, 2002; Ser. No. 10/317,270, filed Dec. 10, 2002; Ser. No. 10/173,902, filed Jun. 17, 2002; Ser. No. 10/174,456, filed Jun. 17, 2002; Ser. No. 10/178,258, filed Jun. 20, 2002; Ser. No. 10/187,659, filed Jul. 1, 2002; Ser. No. 10/211,859, filed Jul. 31, 2002; Ser. No. 10/210,429, filed Jul. 31, 2002; Ser. No. 10/293,998, filed Nov. 11, 2002; Ser. No. 10/316,242, filed Dec. 9, 2002; Ser. No. 10/315,474, filed Dec. 10, 2002; Ser. No. 10/317,803, filed Dec. 11, 2002; Ser. No. 10/346,268, filed Jan. 15, 2003; Ser. No. 10/299,089, filed Nov. 16, 2002; Ser. No. 10/304,082, filed Nov. 22, 2002; Ser. No. 10/304,098, filed Nov. 22, 2002; Ser. No. 10/303,420, filed Nov. 23, 2002; Ser. No. 10/300,236, filed Nov. 19, 2002; Ser. No. 10/300,263, filed Nov. 19, 2002; Ser. No. 10/302,571, filed Nov. 22, 2002; Ser. No. 10/317,478, filed Dec. 11, 2002; Ser. No. 10/301,832, filed Nov. 21, 2002; and each of the above applications are herein incorporated by reference in their entirety.

### FIELD OF THE INVENTION

Disclosed herein are compounds, compositions and methods for modulating the expression of a signal transducer in a cell, tissue or animal.

### BACKGROUND OF THE INVENTION

Targeting disease-causing gene sequences was first suggested more than thirty years ago (Belikova et al., Tet. Lett., 1967, 37, 3557-3562), and antisense activity was demonstrated in cell culture more than a decade later (Zamecnik et al., Proc. Natl. Acad. Sci. U.S.A., 1978, 75, 280-284). One advantage of antisense technology in the treatment of a disease or condition that stems from a disease-causing gene is that it is a direct genetic approach that has the ability to modulate (increase or decrease) the expression of specific disease-causing genes. Another advantage is that validation of a target using antisense compounds results in direct and immediate discovery of the drug candidate; in that the antisense compound is the potential therapeutic agent.

Generally, the principle behind antisense technology is that an antisense compound hybridizes to a target nucleic acid and effects the modulation of gene expression activity, or function, such as transcription or translation. The modulation of gene expression can be achieved by, for example, target degradation or occupancy-based inhibition. An example of modulation of RNA target function by degradation is RNase H-based degradation of the target RNA upon hybridization with a DNA-like antisense compound. Another example of modulation of gene expression by target degradation is RNA interference (RNAi). RNAi generally refers to antisense-mediated gene silencing involving the introduction of dsRNA leading to the sequence-specific reduction of targeted endogenous mRNA levels. Regardless of the specific mechanism, this sequence-specificity makes antisense compounds extremely attractive as tools for target validation and gene functionalization, as well as therapeutics to selectively modulate the expression of genes involved in the pathogenesis of malignancies and other diseases.

Antisense compounds have been employed as therapeutic agents in the treatment of disease states in animals, including humans. Antisense oligonucleotide drugs are being safely and effectively administered to humans in numerous clinical trials. In 1998, the antisense compound, Vitravene® (fomivirsen; developed by Isis Pharmaceuticals Inc., Carlsbad, Calif.) was the first antisense drug to achieve marketing clearance from the U.S. Food and Drug Administration (FDA), and is currently used in the treatment of cytomegalovirus (CMV)-induced retinitis in AIDS patients. A New Drug Application (NDA) for Genasense™ (oblimersen sodium; developed by Genta, Inc., Berkeley Heights, N.J.), an antisense compound which targets the Bcl-2 mRNA overexpressed in many cancers, was accepted by the FDA. Many other antisense compounds are in clinical trials, including those targeting c-myc (NeuGene® AVI-4126, AVI BioPharma, Ridgefield Park, N.J.), TNF-alpha (ISIS 104838, developed by Isis Pharmaceuticals, Inc.), VLA4 (ATL1102, Antisense Therapeutics Ltd., Toorak, Victoria, Australia) and DNA methyltransferase (MG98, developed by MGI Pharma, Bloomington, Minn.).

Chemical modifications have improved the potency and efficacy of antisense compounds, uncovering the potential for oral delivery as well as enhancing subcutaneous administration, decreasing potential for side effects, and leading to improvements in patient convenience. Chemical modifications which increase the potency of antisense compounds allow administration of lower doses, which reduces the potential for toxicity, as well as decreasing overall cost of therapy. Modifications which increase the resistance to degradation result in slower clearance from the body, allowing for less frequent dosing. Various chemical modifications can be combined in one compound to further optimize the compound's efficacy.

Intrinsic, cell-autonomous factors as well as non-autonomous, short-range and long-range signals guide cells through distinct developmental paths. An organism frequently uses the same signaling pathway within different cellular contexts to achieve unique developmental goals.

Notch signaling is an evolutionarily conserved mechanism used to control cell fates through local cell interactions. The gene encoding the original Notch receptor was discovered in Drosophila melanogaster due to the fact that partial loss of function of the gene results in notches at the wing margin (Artavanis-Tsakonas et al., Science, 1999, 284, 770-776). Signals transmitted through the Notch receptor, in combination with other cellular factors, influence differentiation, proliferation and apoptotic events at all stages of development (Artavanis-Tsakonas et al., Science, 1999, 284, 770-776).

Mature Notch proteins are heterodimeric receptors derived from the cleavage of Notch pre-proteins into an extracellular subunit containing multiple EGF-like repeats and a transmembrane subunit including the intracellular region (Blaumueller et al., Cell, 1997, 90, 281-291). Notch activation results from the binding of ligands expressed by neighboring cells or soluble ligands and signaling from activated Notch involves networks of transcription regulators (Artavanis-Tsakonas et al., Science, 1995, 268, 225-232).

In context of experimental cancer immunotherapy, the Notch signaling network is acquiring increasing importance for its possible roles in neoplastic cells and the immune system (Jang et al., Curr. Opin. Mol. Ther., 2000, 2, 55-65).

Four mammalian Notch homologs have been identified and are designated Notch1, Notch2, Notch3 and Notch4. Human Notch2 (also known as Notch (Drosophila homolog 2) was identified and mapped to chromosome 1p13-p11, a region associated with neoplasia-associated translocations (Larsson et al., Genomics, 1994, 24, 253-258). Larsson et al. predicted that the human Notch genes are proto-oncogenes and candidates for sites of chromosome breakage in neoplasia-associated translocations (Larsson et al., Genomics, 1994, 24, 253-258).

Disclosed and claimed in U.S. Pat. No. 5,789,195 are nucleic acid sequences encoding Notch genes. Antibodies to human Notch proteins are additionally provided (Artavanis-Tsakonas et al., 1998). Amino acid sequences of Notch genes and antibodies against Notch proteins are also disclosed and claimed in U.S. Pat. No. 6,090,922 (Artavanis-Tsakonas et al., 2000).

Modulation of expression of Notch genes may prove to be a useful point for therapeutic intervention in developmental, hyperproliferative or autoimmune disorders or disorders arising from aberrant apoptosis.

Methods for producing allergen- or antigen-tolerant T-cells employing compositions capable of upregulating expression of an endogenous Notch protein are disclosed and claimed in PCT publication WO 00/36089 (Lamb et al., 2000).

Disclosed and claimed in U.S. Pat. No. 6,149,902 is a method for cell transplantation which includes contacting a precursor cell with an agonist of Notch function effective to inhibit differentiation of the cell wherein said agonist is a Delta protein, a Serrate protein or an antibody to a Notch protein (Artavanis-Tsakonas et al., 2000).

Disclosed in U.S. Pat. No. 6,083,904 and PCT publication WO 94/07474 are therapeutic and diagnostic methods and compositions based on Notch proteins and nucleic acids, wherein antisense methods are generally disclosed (Artavanis-Tsakonas, 2000; Artavanis-Tsakonas et al., 1994).

Disclosed and claimed in U.S. Pat. No. 5,786,158 are methods and compositions for the detection of malignancy or nervous system disorders based on the level of Notch proteins or nucleic acids (Artavanis-Tsakonas et al., 1998).

Disclosed and claimed in PCT publication WO 00/20576 are methods for inducing differentiation and apoptosis in human cells that over express Notch proteins wherein Notch function is disrupted using antisense oligonucleotides that target the EGF repeat region, the lin/notch region and the ankyrin region (Miele et al., 2000).

Disclosed and claimed in PCT publication WO 01/25422 is an antisense oligonucleotide directed to exon 22 of human Notch2 (Bartelmez and Iversen, 2001).

Currently, there are no known therapeutic agents that effectively inhibit the synthesis of Notch2. To date, investigative strategies aimed at modulating Notch2 expression have involved the use of antibodies and Notch-regulating proteins as well as antisense RNA and oligonucleotides. Consequently, there remains a long felt need for additional agents capable of effectively inhibiting Notch2 function.

Antisense technology is an effective means for reducing the expression of one or more specific gene products and is uniquely useful in a number of therapeutic, diagnostic, and research applications.

Disclosed herein are antisense compounds useful for modulating gene expression and associated pathways via antisense mechanisms of action such as RNaseH, RNAi and dsRNA enzymes, as well as other antisense mechanisms based on target degradation or target occupancy. One having skill in the art, once armed with this disclosure will be able, without undue experimentation, to identify, prepare and exploit antisense compounds for these uses.

### SUMMARY OF THE INVENTION

Provided herein are oligomeric compounds, especially nucleic acid and nucleic acid-like oligomers, which are targeted to a nucleic acid encoding a signal transducer. Signal transducers disclosed herein include endothelial differentiation gene 2, Notch2, junctional adhesion molecule 3, tumor endothelial marker 5 precursor, G protein-coupled receptor 6, G protein-coupled receptor 12, vascular endothelial growth factor B, ephrin-B2, zinedin, G protein-coupled receptor 39, G protein-coupled receptor 49, heme oxygenase 1, P2×4, Ran GTPase activating protein 1, hepatoma-derived growth factor, G protein-coupled receptor RE2, RP105-associated MD-1, G protein-coupled receptor 3, angiopoietin-2, thyroid hormone receptor interactor 3, interleukin 22, jagged 1, coagulation factor III, B7H, CD24, estrogen-related receptor beta like 1, heat shock 90 kD protein 1 alpha, interleukin 18 and Notch 3.

The present invention is directed to antisense compounds, especially nucleic acid and nucleic acid-like oligomers, which are targeted to a nucleic acid encoding Notch2, and which modulate the expression of Notch2. Pharmaceutical and other compositions comprising the compounds of the invention are also provided. Further provided are methods of screening for modulators of Notch2 and methods of modulating the expression of Notch2 in cells, tissues or animals comprising contacting said cells, tissues or animals with one or more of the compounds or compositions of the invention. Methods of treating an animal, particularly a human, suspected of having or being prone to a disease or condition associated with expression of Notch2 are also set forth herein. Such methods comprise administering a therapeutically or prophylactically effective amount of one or more of the compounds or compositions of the invention to the person in need of treatment.

### DETAILED DESCRIPTION OF THE INVENTION

A. Overview of the Invention

The present invention employs antisense compounds, preferably oligonucleotides and similar species for use in modulating the function or effect of nucleic acid molecules encoding Notch2. This is accomplished by providing oligonucleotides which specifically hybridize with one or more nucleic acid molecules encoding Notch2. As used herein, the terms “target nucleic acid” and “nucleic acid molecule encoding Notch2” have been used for convenience to encompass DNA encoding Notch2, RNA (including pre-mRNA and mRNA or portions thereof) transcribed from such DNA, and also cDNA derived from such RNA. The hybridization of a compound of this invention with its target nucleic acid is generally referred to as “antisense”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. One preferred result of such interference with target nucleic acid function is modulation of the expression of Notch2. In the context of the present invention, “modulation” and “modulation of expression” mean either an increase (stimulation) or a decrease (inhibition) in the amount or levels of a nucleic acid molecule encoding the gene, e.g., DNA or RNA. Inhibition is often the preferred form of modulation of expression and mRNA is often a preferred target nucleic acid.

In the context of this invention, “hybridization” means the pairing of complementary strands of oligomeric compounds. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

In the present invention the phrase “stringent hybridization conditions” or “stringent conditions” refers to conditions under which a compound of the invention will hybridize to its target sequence, but to a minimal number of other sequences. Stringent conditions are sequence-dependent and will be different in different circumstances and in the context of this invention, “stringent conditions” under which oligomeric compounds hybridize to a target sequence are determined by the nature and composition of the oligomeric compounds and the assays in which they are being investigated.

“Complementary,” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

It is understood in the art that the sequence of an antisense compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure or hairpin structure). It is preferred that the antisense compounds of the present invention comprise at least 70%, or at least 75%, or at least 80%, or at least 85% sequence complementarity to a target region within the target nucleic acid, more preferably that they comprise at least 90% sequence complementarity and even more preferably comprise at least 95% or at least 99% sequence complementarity to the target region within the target nucleic acid sequence to which they are targeted. For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some embodiments, homology, sequence identity or complementarity, between the oligomeric and target is between about 50% to about 60%. In some embodiments, homology, sequence identity or complementarity, is between about 60% to about 70%. In further embodiments, homology, sequence identity or complementarity, is between about 70% and about 80%. In further embodiments, homology, sequence identity or complementarity, is between about 80% and about 90%. In some preferred embodiments, homology, sequence identity or complementarity, is about 90%, about 92%, about 94%, about 95%, about 96%, about 97%, about 98%, about 99% or about 100%.

B. Compounds of the Invention

According to the present invention, antisense compounds include antisense oligomeric compounds, antisense oligonucleotides, siRNAs, external guide sequence (EGS) oligonucleotides, alternate splicers, and other oligomeric compounds which hybridize to at least a portion of the target nucleic acid. As such, these compounds may be introduced in the form of single-stranded, double-stranded, circular or hairpin oligomeric compounds and may contain structural elements such as internal or terminal bulges or loops. Once introduced to a system, the compounds of the invention may elicit the action of one or more enzymes or structural proteins to effect modification of the target nucleic acid.

One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While the one form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing.

The first evidence that dsRNA could lead to gene silencing in animals came in 1995 from work in the nematode, Caenorhabditis elegans (Guo and Kempheus, Cell, 1995, 81, 611-620). Montgomery et al. have shown that the primary interference effects of dsRNA are posttranscriptional (Montgomery et al., Proc. Natl. Acad. Sci. USA, 1998, 95, 15502-15507). The posttranscriptional antisense mechanism defined in Caenorhabditis elegans resulting from exposure to double-stranded RNA (dsRNA) has since been designated RNA interference (RNAi). This term has been generalized to mean antisense-mediated gene silencing involving the introduction of dsRNA leading to the sequence-specific reduction of endogenous targeted mRNA levels (Fire et al., Nature, 1998, 391, 806-811). Recently, it has been shown that it is, in fact, the single-stranded RNA oligomers of antisense polarity of the dsRNAs which are the potent inducers of RNAi (Tijsterman et al., Science, 2002, 295, 694-697).

The antisense compounds of the present invention also include modified compounds in which a different base is present at one or more of the nucleotide positions in the compound. For example, if the first nucleotide is an adenosine, modified compounds may be produced which contain thymidine, guanosine or cytidine at this position. This may be done at any of the positions of the antisense compound. These compounds are then tested using the methods described herein to determine their ability to inhibit expression of Notch2 mRNA.

In the context of this invention, the term “oligomeric compound” refers to a polymer or oligomer comprising a plurality of monomeric units. In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

While oligonucleotides are a preferred form of the antisense compounds of this invention, the present invention comprehends other families of antisense compounds as well, including but not limited to oligonucleotide analogs and mimetics such as those described herein.

The antisense compounds in accordance with this invention preferably comprise from about 8 to about 80 nucleobases (i.e. from about 8 to about 80 linked nucleosides). One of ordinary skill in the art will appreciate that the invention embodies compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

In one embodiment, the antisense compounds of the invention are 12 to 50 nucleobases in length. One having ordinary skill in the art will appreciate that this embodies compounds of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length.

In one embodiment, the antisense compounds of the invention are 13 to 40 nucleobases in length. One having ordinary skill in the art will appreciate that this embodies compounds of 13, 14, 15, 16, 17, 18, 19, 20, 21, 22, 23, 24, 25, 26, 27, 28, 29, 30, 31, 32, 33, 34, 35, 36, 37, 38, 39 or 40 nucleobases in length.

In another embodiment, the antisense compounds of the invention are 15 to 30 nucleobases in length. One having ordinary skill in the art will appreciate that this embodies compounds of 15, 16, 17, 18, 19, 20, 21, 22, 23, 24, 25, 26, 27, 28, 29, or 30 nucleobases in length.

Particular compounds are oligonucleotides from about 12 to about 50 nucleobases, from about 13 to about 40 nucleobases, even more preferably those comprising from about 15 to about 30 nucleobases.

Antisense compounds 8-80 nucleobases in length comprising a stretch of at least eight (8) consecutive nucleobases selected from within the illustrative antisense compounds are considered to be suitable antisense compounds as well.

Exemplary antisense compounds include oligonucleotide sequences that comprise at least the 8 consecutive nucleobases from the 5′-terminus of one of the illustrative preferred antisense compounds (the remaining nucleobases being a consecutive stretch of the same oligonucleotide beginning immediately upstream of the 5′-terminus of the antisense compound which is specifically hybridizable to the target nucleic acid and continuing until the oligonucleotide contains about 8 to about 80 nucleobases). Similarly preferred antisense compounds are represented by oligonucleotide sequences that comprise at least the 8 consecutive nucleobases from the 3′-terminus of one of the illustrative preferred antisense compounds (the remaining nucleobases being a consecutive stretch of the same oligonucleotide beginning immediately downstream of the 3′-terminus of the antisense compound which is specifically hybridizable to the target nucleic acid and continuing until the oligonucleotide contains about 8 to about 80 nucleobases). It is also understood that preferred antisense compounds may be represented by oligonucleotide sequences that comprise at least 8 consecutive nucleobases from an internal portion of the sequence of an illustrative preferred antisense compound, and may extend in either or both directions until the oligonucleotide contains about 8 to about 80 nucleobases.

One having skill in the art armed with the preferred antisense compounds illustrated herein will be able, without undue experimentation, to identify further preferred antisense compounds.

C. Targets of the Invention

“Targeting” an antisense compound to a particular nucleic acid molecule, in the context of this invention, can be a multistep process. The process usually begins with the identification of a target nucleic acid whose function is to be modulated. This target nucleic acid may be, for example, a cellular gene (or mRNA transcribed from the gene) whose expression is associated with a particular disorder or disease state, or a nucleic acid molecule from an infectious agent. In the present invention, the target nucleic acid encodes Notch2.

The targeting process usually also includes determination of at least one target region, segment, or site within the target nucleic acid for the antisense interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid.

Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA transcribed from a gene encoding Notch2, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the antisense compounds of the present invention.

The open reading frame (ORF) or “coding region,” which is known in the art to refer to the region between the translation initiation codon and the translation termination codon, is also a region which may be targeted effectively. Within the context of the present invention, a preferred region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence, resulting in exon-exon junctions at the sites where exons are joined. Targeting exon-exon junctions can be useful in situations where the overproduction of a normal splice product is implicated in disease, or where the overproduction of an aberrant splice product is implicated in disease. Targeting splice sites, i.e., intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also preferred target sites. mRNA transcripts produced via the process of splicing of two (or more) mRNAs from different gene sources known as “fusion transcripts” are also suitable target sites. It is also known that introns can be effectively targeted using antisense compounds targeted to, for example, DNA or pre-mRNA.

It is also known in the art that alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and exonic sequence.

Upon excision of one or more exon or intron regions, or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Within the context of the invention, the types of variants described herein are also preferred target nucleic acids.

The locations on the target nucleic acid to which the preferred antisense compounds hybridize are hereinbelow referred to as “preferred target segments.” As used herein the term “preferred target segment” is defined as at least an 8-nucleobase portion of a target region to which an active antisense compound is targeted. While not wishing to be bound by theory, it is presently believed that these target segments represent portions of the target nucleic acid which are accessible for hybridization.

While the specific sequences of certain preferred target segments are set forth herein, one of skill in the art will recognize that these serve to illustrate and describe particular embodiments within the scope of the present invention. Additional preferred target segments may be identified by one having ordinary skill.

Target segments 8-80 nucleobases in length comprising a stretch of at least eight (8) consecutive nucleobases selected from within the illustrative preferred target segments are considered to be suitable for targeting as well.

Target segments can include DNA or RNA sequences that comprise at least the 8 consecutive nucleobases from the 5′-terminus of one of the illustrative preferred target segments (the remaining nucleobases being a consecutive stretch of the same DNA or RNA beginning immediately upstream of the 5′-terminus of the target segment and continuing until the DNA or RNA contains about 8 to about 80 nucleobases). Similarly preferred target segments are represented by DNA or RNA sequences that comprise at least the 8 consecutive nucleobases from the 3′-terminus of one of the illustrative preferred target segments (the remaining nucleobases being a consecutive stretch of the same DNA or RNA beginning immediately downstream of the 3′-terminus of the target segment and continuing until the DNA or RNA contains about 8 to about 80 nucleobases). It is also understood that preferred antisense target segments may be represented by DNA or RNA sequences that comprise at least 8 consecutive nucleobases from an internal portion of the sequence of an illustrative preferred target segment, and may extend in either or both directions until the oligonucleotide contains about 8 to about 80 nucleobases. One having skill in the art armed with the preferred target segments illustrated herein will be able, without undue experimentation, to identify further preferred target segments.

Once one or more target regions, segments or sites have been identified, antisense compounds are chosen which are sufficiently complementary to the target, i.e., hybridize sufficiently well and with sufficient specificity, to give the desired effect.

The oligomeric antisense compounds can also be targeted to regions of a target nucleobase sequence, such as those disclosed herein (e.g. in Example 13). All regions of a nucleobase sequence to which an oligomeric antisense compound can be targeted, wherein the regions are greater than or equal to 8 and less than or equal to 80 nucleobases, are described as follows:

Let R(n, n+m−1) be a region from a target nucleobase sequence, where “n” is the 5′-most nucleobase position of the region, where “n+m−1” is the 3′-most nucleobase position of the region and where “m” is the length of the region. A set “S(m)”, of regions of length “m” is defined as the regions where n ranges from 1 to L−m+1, where L is the length of the target nucleobase sequence and L>m. A set, “A”, of all regions can be constructed as a union of the sets of regions for each length from where m is greater than or equal to 8 and is less than or equal to 80.

This set of regions can be represented using the following mathematical notation:

$$A = {Y}_{m} S ( m ) where m ∈ N \vert 8 \leq m \leq 80 and$$

$$S ( m ) = \{ {R}_{n , n + m - 1} \vert n ∈ \{ 1 , 2 , 3 , … , L - m + 1 \} \}$$

where the mathematical operator | indicates “such that”,
where the mathematical operator ε indicates “a member of a set” (e.g. yεZ indicates that element y is a member of set Z),
where x is a variable,
where N indicates all natural numbers, defined as positive integers,
and where the mathematical operator Y indicates “the union of sets”.

For example, the set of regions for m equal to 8, 20 and 80 can be constructed in the following manner. The set of regions, each 8 nucleobases in length, S(m=8), in a target nucleobase sequence 100 nucleobases in length (L=100), beginning at position 1 (n=1) of the target nucleobase sequence, can be created using the following expression:
S(8)={R_{1,8}|nε{1, 2, 3, . . . , 93}}
and describes the set of regions comprising nucleobases 1-8, 2-9, 3-10, 4-11, 5-12, 6-13, 7-14, 8-15, 9-16, 10-17, 11-18, 12-19, 13-20, 14-21, 15-22, 16-23, 17-24, 18-25, 19-26, 20-27, 21-28, 22-29, 23-30, 24-31, 25-32, 26-33, 27-34, 28-35, 29-36, 30-37, 31-38, 32-39, 33-40, 34-41, 35-42, 36-43, 37-44, 38-45, 39-46, 40-47, 41-48, 42-49, 43-50, 44-51, 45-52, 46-53, 47-54, 48-55, 49-56, 50-57, 51-58, 52-59, 53-60, 54-61, 55-62, 56-63, 57-64, 58-65, 59-66, 60-67, 61-68, 62-69, 63-70, 64-71, 65-72, 66-73, 67-74, 68-75, 69-76, 70-77, 71-78, 72-79, 73-80, 74-81, 75-82, 76-83, 77-84, 78-85, 79-86, 80-87, 81-88, 82-89, 83-90, 84-91, 85-92, 86-93, 87-94, 88-95, 89-96, 90-97, 91-98, 92-99, 93-100.

An additional set for regions 20 nucleobases in length, in a target sequence 100 nucleobases in length, beginning at position 1 of the target nucleobase sequence, can be described using the following expression:
S(20)={R_{1,20}|nε{1, 2, 3, . . . , 81}}
and describes the set of regions comprising nucleobases 1-20, 2-21, 3-22, 4-23, 5-24, 6-25, 7-26, 8-27, 9-28, 10-29, 11-30, 12-31, 13-32, 14-33, 15-34, 16-35, 17-36, 18-37, 19-38, 20-39, 21-40, 22-41, 23-42, 24-43, 25-44, 26-45, 27-46, 28-47, 29-48, 30-49, 31-50, 32-51, 33-52, 34-53, 35-54, 36-55, 37-56, 38-57, 39-58, 40-59, 41-60, 42-61, 43-62, 44-63, 45-64, 46-65, 47-66, 48-67, 49-68, 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 75-94, 76-95, 77-96, 78-97, 79-98, 80-99, 81-100.

An additional set for regions 80 nucleobases in length, in a target sequence 100 nucleobases in length, beginning at position 1 of the target nucleobase sequence, can be described using the following expression:
S(80)={R_{1,80}|nε{1, 2, 3, . . . , 21}}
and describes the set of regions comprising nucleobases 1-80, 2-81, 3-82, 4-83, 5-84, 6-85, 7-86, 8-87, 9-88, 10-89, 11-90, 12-91, 13-92, 14-93, 15-94, 16-95, 17-96, 18-97, 19-98, 20-99, 21-100.

Thus, in this example, A would include regions 1-8, 2-9, 3-10 . . . 93-100, 1-20, 2-21, 3-22 . . . 81-100, 1-80, 2-81, 3-82 . . . 21-100.

The union of these aforementioned example sets and other sets for lengths from 10 to 19 and 21 to 79 can be described using the mathematical expression

$$A = {Y}_{m} S ( m )$$

where Y represents the union of the sets obtained by combining all members of all sets.

The mathematical expressions described herein defines all possible target regions in a target nucleobase sequence of any length L, where the region is of length m, and where m is greater than or equal to 8 and less than or equal to 80 nucleobases and, and where m is less than L, and where n is less than L−m+1.

D. Screening and Target Validation

In a further embodiment, the “preferred target segments” identified herein may be employed in a screen for additional compounds that modulate the expression of Notch2. “Modulators” are those compounds that decrease or increase the expression of a nucleic acid molecule encoding Notch2 and which comprise at least an 8-nucleobase portion which is complementary to a preferred target segment. The screening method comprises the steps of contacting a preferred target segment of a nucleic acid molecule encoding Notch2 with one or more candidate modulators, and selecting for one or more candidate modulators which decrease or increase the expression of a nucleic acid molecule encoding Notch2. Once it is shown that the candidate modulator or modulators are capable of modulating (e.g. either decreasing or increasing) the expression of a nucleic acid molecule encoding Notch2, the modulator may then be employed in further investigative studies of the function of Notch2, or for use as a research, diagnostic, or therapeutic agent in accordance with the present invention.

The preferred target segments of the present invention may be also be combined with their respective complementary antisense compounds of the present invention to form stabilized double-stranded (duplexed) oligonucleotides.

Such double stranded oligonucleotide moieties have been shown in the art to modulate target expression and regulate translation as well as RNA processsing via an antisense mechanism. Moreover, the double-stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200). For example, such double-stranded moieties have been shown to inhibit the target by the classical hybridization of antisense strand of the duplex to the target, thereby triggering enzymatic degradation of the target (Tijsterman et al., Science, 2002, 295, 694-697).

The antisense compounds of the present invention can also be applied in the areas of drug discovery and target validation. The present invention comprehends the use of the compounds and preferred target segments identified herein in drug discovery efforts to elucidate relationships that exist between Notch2 and a disease state, phenotype, or condition. These methods include detecting or modulating Notch2 comprising contacting a sample, tissue, cell, or organism with the compounds of the present invention, measuring the nucleic acid or protein level of Notch2 and/or a related phenotypic or chemical endpoint at some time after treatment, and optionally comparing the measured value to a non-treated sample or sample treated with a further compound of the invention. These methods can also be performed in parallel or in combination with other experiments to determine the function of unknown genes for the process of target validation or to determine the validity of a particular gene product as a target for treatment or prevention of a particular disease, condition, or phenotype.

E. Kits, Research Reagents, Diagnostics, and Therapeutics

The antisense compounds of the present invention can be utilized for diagnostics, therapeutics, prophylaxis and as research reagents and kits. Furthermore, antisense oligonucleotides, which are able to inhibit gene expression with exquisite specificity, are often used by those of ordinary skill to elucidate the function of particular genes or to distinguish between functions of various members of a biological pathway.

For use in kits and diagnostics, the compounds of the present invention, either alone or in combination with other compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more antisense compounds are compared to control cells or tissues not treated with antisense compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (To, Comb. Chem. High Throughput Screen, 2000, 3, 235-41).

The antisense compounds of the invention are useful for research and diagnostics, because these compounds hybridize to nucleic acids encoding Notch2. The primers and probes disclosed herein are useful in methods requiring the specific detection of nucleic acid molecules encoding Notch2 and in the amplification of said nucleic acid molecules for detection or for use in further studies of Notch2. Hybridization of the primers and probes with a nucleic acid encoding Notch2 can be detected by means known in the art. Such means may include conjugation of an enzyme to the primer or probe, radiolabelling of the primer or probe or any other suitable detection means. Kits using such detection means for detecting the level of Notch2 in a sample may also be prepared.

The specificity and sensitivity of antisense is also harnessed by those of skill in the art for therapeutic uses. Antisense compounds have been employed as therapeutic moieties in the treatment of disease states in animals, including humans. Antisense oligonucleotide drugs, including ribozymes, have been safely and effectively administered to humans and numerous clinical trials are presently underway. It is thus established that antisense compounds can be useful therapeutic modalities that can be configured to be useful in treatment regimes for the treatment of cells, tissues and animals, especially humans.

For therapeutics, an animal, preferably a human, suspected of having a disease or disorder which can be treated by modulating the expression of Notch2 is treated by administering antisense compounds in accordance with this invention. For example, in one non-limiting embodiment, the methods comprise the step of administering to the animal in need of treatment, a therapeutically effective amount of a Notch2 inhibitor. The Notch2 inhibitors of the present invention effectively inhibit the activity of the Notch2 protein or inhibit the expression of the Notch2 protein. In one embodiment, the activity or expression of Notch2 in an animal is inhibited by about 10%. Preferably, the activity or expression of Notch2 in an animal is inhibited by about 30%. More preferably, the activity or expression of Notch2 in an animal is inhibited by 50% or more. Thus, the oligomeric antisense compounds modulate expression of Notch2 mRNA by at least 10%, by at least 20%, by at least 25%, by at least 30%, by at least 40%, by at least 50%, by at least 60%, by at least 70%, by at least 75%, by at least 80%, by at least 85%, by at least 90%, by at least 95%, by at least 98%, by at least 99%, or by 100%.

For example, the reduction of the expression of Notch2 may be measured in serum, adipose tissue, liver or any other body fluid, tissue or organ of the animal. Preferably, the cells contained within said fluids, tissues or organs being analyzed contain a nucleic acid molecule encoding Notch2 protein and/or the Notch2 protein itself.

The antisense compounds of the invention can be utilized in pharmaceutical compositions by adding an effective amount of a compound to a suitable pharmaceutically acceptable diluent or carrier. Use of the compounds and methods of the invention may also be useful prophylactically.

F. Modifications

As is known in the art, a nucleoside is a base-sugar combination. The base portion of the nucleoside is normally a heterocyclic base sometimes referred to as a “nucleobase” or simply a “base”. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Modified Internucleoside Linkages (Backbones)

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphorodithioates, phosphotriesters, aminoalkylphosphotriaminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

Modified Sugar and Internucleoside Linkages-Mimetics

In other preferred antisense compounds, e.g., oligonucleotide mimetics, both the sugar and the internucleoside linkage (i.e. the backbone), of the nucleotide units are replaced with novel groups. The nucleobase units are maintained for hybridization with an appropriate target nucleic acid. One such compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Further embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified Sugars

Modified antisense compounds may also contain one or more substituted sugar moieties. Preferred are antisense compounds, preferably antisense oligonucleotides, comprising one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-O-methoxyethyl (2′-O—CH₂CH₂OCH₃, also known as 2′-O-(2-methoxyethyl) or 2′-methoxyethoxy or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—(CH₂)₂—O—(CH₂)₂—N(CH₃)₂, also described in examples hereinbelow.

Other modifications include 2′-methoxy (2′-O—CH₃), 2′-aminopropoxy (2′-OCH₂CH₂CH₂NH₂), 2′-allyl (2′-CH₂—CH═CH₂), 2′®-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Antisense compounds may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is herein incorporated by reference in its entirety.

A further modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is preferably a methylene (—CH₂—)ₙ group bridging the 2′ oxygen atom and the 4′ carbon atom wherein n is 1 or 2. LNAs and preparation thereof are described in WO 98/39352 and WO 99/14226.

Natural and Modified Nucleobases

Antisense compounds may also include nucleobase (often referred to in the art as heterocyclic base or simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, each of which is herein incorporated by reference, and U.S. Pat. No. 5,750,692, also herein incorporated by reference.

Conjugates

Another modification of the antisense compounds of the invention involves chemically linking to the antisense compound one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application PCT/US92/09196, filed Oct. 23, 1992, and U.S. Pat. No. 6,287,860, the entire disclosures of which are incorporated herein by reference. Conjugate moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. Antisense compounds of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

Oligomeric compounds used in the compositions of the present invention can also be modified to have one or more stabilizing groups that are generally attached to one or both termini of oligomeric compounds to enhance properties such as for example nuclease stability. Included in stabilizing groups are cap structures. By “cap structure or terminal cap moiety” is meant chemical modifications, which have been incorporated at either terminus of oligonucleotides (see for example Wincott et al., WO 97/26270, incorporated by reference herein). These terminal modifications protect the oligomeric compounds having terminal nucleic acid molecules from exonuclease degradation, and can help in delivery and/or localization within a cell. The cap can be present at the 5′-terminus (5′-cap) or at the 3′-terminus (3′-cap) or can be present on both termini. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl riucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

Particularly preferred 3′-cap structures of the present invention include, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Tyer, 1993, Tetrahedron 49, 1925; incorporated by reference herein).

Further 3′ and 5′-stabilizing groups that can be used to cap one or both ends of an oligomeric compound to impart nuclease stability include those disclosed in WO 03/004602 published on Jan. 16, 2003.

Chimeric Compounds

It is not necessary for all positions in a given compound to be uniformly modified, and in fact more than one of the aforementioned modifications may be incorporated in a single compound or even at a single nucleoside within an oligonucleotide.

The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. Chimeric antisense oligonucleotides are thus a form of antisense compound. These oligonucleotides typically contain at least one region wherein the oligonucleotide is modified so as to confer upon the oligonucleotide increased resistance to nuclease degradation, increased cellular uptake, increased stability and/or increased binding affinity for the target nucleic acid. An additional region of the oligonucleotide may serve as a substrate for enzymes capable of cleaving RNA:DNA or RNA:RNA hybrids. By way of example, RNAse H is a cellular endonuclease which cleaves the RNA strand of an RNA:DNA duplex. Activation of RNase H, therefore, results in cleavage of the RNA target, thereby greatly enhancing the efficiency of oligonucleotide-mediated inhibition of gene expression. The cleavage of RNA:RNA hybrids can, in like fashion, be accomplished through the actions of endoribonucleases, such as RNAseL which cleaves both cellular and viral RNA. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Chimeric antisense compounds can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”. Such compounds have also been referred to in the art as hybrids. In a gapmer that is 20 nucleotides in length, a gap or wing can be 1, 2, 3, 4, 5, 6, 7, 8, 9, 10, 11, 12, 13, 14, 15, 16, 17 or 18 nucleotides in length. In one embodiment, a 20-nucleotide gapmer is comprised of a gap 8 nucleotides in length, flanked on both the 5′ and 3′ sides by wings 6 nucleotides in length. In another embodiment, a 20-nucleotide gapmer is comprised of a gap 10 nucleotides in length, flanked on both the 5′ and 3′ sides by wings 5 nucleotides in length. In another embodiment, a 20-nucleotide gapmer is comprised of a gap 12 nucleotides in length flanked on both the 5′ and 3′ sides by wings 4 nucleotides in length. In a further embodiment, a 20-nucleotide gapmer is comprised of a gap 14 nucleotides in length flanked on both the 5′ and 3′ sides by wings 3 nucleotides in length. In another embodiment, a 20-nucleotide gapmer is comprised of a gap 16 nucleotides in length flanked on both the 5′ and 3′ sides by wings 2 nucleotides in length. In a further embodiment, a 20-nucleotide gapmer is comprised of a gap 18 nucleotides in length flanked on both the 5′ and 3′ ends by wings 1 nucleotide in length. Alternatively, the wings are of different lengths, for example, a 20-nucleotide gapmer may be comprised of a gap 10 nucleotides in length, flanked by a 6-nucleotide wing on one side (5′ or 3′) and a 4-nucleotide wing on the other side (5′ or 3′).

In a hemimer, an “open end” chimeric antisense compound having two chemically distinct regions, a first chemically distinct region, or the gap segment, in a compound 20 nucleotides in length can be located at the 5′ terminus of the oligomeric compound and can be 1, 2, 3, 4, 5, 6, 7, 8, 9, 10, 11, 12, 13, 14, 15, 16, 17, 18 or 19 nucleotides in length. Furthermore, a second chemically distinct region in a compound 20 nucleotides in length can be located at the 3′ terminus of the oligomeric compound and can be 1, 2, 3, 4, 5, 6, 7, 8, 9, 10, 11, 12, 13, 14, 15, 16, 17, 18 or 19 nucleotides in length. For example, a 20-nucleotide hemimer can have a first chemically distinct region, or a gap segment, of 10 nucleotides at the 5′ end and a second chemically distinct region of 10 nucleotides at the 3′ end.

Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

G. Formulations

The compounds of the invention may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal, including a human, is capable of providing (directly or indirectly) the biologically active metabolite or residue thereof.

The term “pharmaceutically acceptable salts” refers to physiologically and pharmaceutically acceptable salts of the compounds of the invention: i.e., salts that retain the desired biological activity of the parent compound and do not impart undesired toxicological effects thereto. For oligonucleotides, preferred examples of pharmaceutically acceptable salts and their uses are further described in U.S. Pat. No. 6,287,860, which is incorporated herein in its entirety.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the present invention include, but are not limited to, solutions, emulsions, foams and liposome-containing formulations. The pharmaceutical compositions and formulations of the present invention may comprise one or more penetration enhancers, carriers, excipients or other active or inactive ingredients.

Emulsions are typically heterogenous systems of one liquid dispersed in another in the form of droplets usually exceeding 0.1 μm in diameter. Emulsions may contain additional components in addition to the dispersed phases, and the active drug which may be present as a solution in either the aqueous phase, oily phase or itself as a separate phase. Microemulsions are included as an embodiment of the present invention. Emulsions and their uses are well known in the art and are further described in U.S. Pat. No. 6,287,860, which is incorporated herein in its entirety.

Formulations of the present invention include liposomal formulations. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety.

The pharmaceutical formulations and compositions of the present invention may also include surfactants. The use of surfactants in drug products, formulations and in emulsions is well known in the art. Surfactants and their uses are further described in U.S. Pat. No. 6,287,860, which is incorporated herein in its entirety.

In one embodiment, the present invention employs various penetration enhancers to effect the efficient delivery of nucleic acids, particularly oligonucleotides. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that formulations are routinely designed according to their intended use, i.e. route of administration.

Preferred formulations for topical administration include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, oligonucleotides of the invention may be encapsulated within liposomes or may form complexes thereto, in particular to cationic liposomes. Alternatively, oligonucleotides may be complexed to lipids, in particular to cationic lipids. Preferred fatty acids and esters, pharmaceutically acceptable salts thereof, and their uses are further described in U.S. Pat. No. 6,287,860, which is incorporated herein in its entirety. Topical formulations are described in detail in U.S. patent application Ser. No. 09/315,298 filed on May 20, 1999, which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts and fatty acids and their uses are further described in U.S. Pat. No. 6,287,860, which is incorporated herein in its entirety. Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally, in granular form including sprayed dried particles, or complexed to form micro or nanoparticles. Oligonucleotide complexing agents and their uses are further described in U.S. Pat. No. 6,287,860, which is incorporated herein in its entirety. Oral formulations for oligonucleotides and their preparation are described in detail in U.S. application Ser. No. 09/108,673 (filed Jul. 1, 1998), Ser. No. 09/315,298 (filed May 20, 1999) and Ser. No. 10/071,822, filed Feb. 8, 2002, each of which is incorporated herein by reference in their entirety.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Oligonucleotides may be formulated for delivery in vivo in an acceptable dosage form, e.g. as parenteral or non-parenteral formulations. Parenteral formulations include intravenous (IV), subcutaneous (SC), intraperitoneal (IP), intravitreal and intramuscular (IM) formulations, as well as formulations for delivery via pulmonary inhalation, intranasal administration, topical administration, etc. Non-parenteral formulations include formulations for delivery via the alimentary canal, e.g. oral administration, rectal administration, intrajejunal instillation, etc. Rectal administration includes administration as an enema or a suppository. Oral administration includes administration as a capsule, a gel capsule, a pill, an elixir, etc.

In some embodiments, an oligonucleotide may be administered to a subject via an oral route of administration. The subjects of the present invention comprise animals. An animal subject may be a mammal, such as a mouse, a rat, a dog, a guinea pig, a cat, a pig or a non-human primate. Non-human primates include monkeys and chimpanzees. A suitable animal subject may be an experimental animal, such as a mouse, a rat, a cat, a pig or non-human primate.

In some embodiments, the subject may be a human. In certain embodiments, the subject may be a human patient in need of therapeutic treatment as discussed in more detail herein. In certain embodiments, the subject may be in need of modulation of expression of one or more genes as discussed in more detail herein. In some particular embodiments, the subject may be in need of inhibition of expression of one or more genes as discussed in more detail herein. In particular embodiments, the subject may be in need of modulation, i.e. inhibition or enhancement, of Notch2 in order to obtain therapeutic indications discussed in more detail herein.

In some embodiments, non-parenteral (e.g. oral) oligonucleotide formulations according to the present invention result in enhanced bioavailability of the oligonucleotide. In this context, the term “bioavailability” refers to a measurement of that portion of an administered drug which reaches the circulatory system (e.g. blood, especially blood plasma) when a particular mode of administration is used to deliver the drug. Enhanced bioavailability refers to a particular mode of administration's ability to deliver oligonucleotide to the peripheral blood plasma of a subject relative to another mode of administration. For example, when a non-parenteral mode of administration (e.g. an oral mode) is used to introduce the drug into a subject, the bioavailability for that mode of administration may be compared to a different mode of administration, e.g. an IV mode of administration. In some embodiments, the area under a compound's blood plasma concentration curve (AUC₀) after non-parenteral (e.g. oral, rectal, intrajejunal) administration may be divided by the area under the drug's plasma concentration curve after intravenous (i.v.) administration (AUCᵢᵥ) to provide a dimensionless quotient (relative bioavailability, RB) that represents fraction of compound absorbed via the non-parenteral route as compared to the IV route. A composition's bioavailability is said to be enhanced in comparison to another composition's bioavailability when the first composition's relative bioavailability (RB₁) is greater than the second composition's relative bioavailability (RB₂).

In general, bioavailability correlates with therapeutic efficacy when a compound's therapeutic efficacy is related to the blood concentration achieved, even if the drug's ultimate site of action is intracellular (van Berge-Henegouwen et al., Gastroenterol., 1977, 73, 300). Bioavailability studies have been used to determine the degree of intestinal absorption of a drug by measuring the change in peripheral blood levels of the drug after an oral dose (DiSanto, Chapter 76 In: Remington's Pharmaceutical Sciences, 18th Ed., Gennaro, ed., Mack Publishing Co., Easton, Pa., 1990, pages 1451-1458).

In general, an oral composition's bioavailability is said to be “enhanced” when its relative bioavailability is greater than the bioavailability of a composition substantially consisting of pure oligonucleotide, i.e. oligonucleotide in the absence of a penetration enhancer.

Organ bioavailability refers to the concentration of compound in an organ. Organ bioavailability may be measured in test subjects by a number of means, such as by whole-body radiography. Organ bioavailability may be modified, e.g. enhanced, by one or more modifications to the oligonucleotide, by use of one or more carrier compounds or excipients, etc. as discussed in more detail herein. In general, an increase in bioavailability will result in an increase in organ bioavailability.

Oral oligonucleotide compositions according to the present invention may comprise one or more “mucosal penetration enhancers,” also known as “absorption enhancers” or simply as “penetration enhancers.” Accordingly, some embodiments of the invention comprise at least one oligonucleotide in combination with at least one penetration enhancer. In general, a penetration enhancer is a substance that facilitates the transport of a drug across mucous membrane(s) associated with the desired mode of administration, e.g. intestinal epithelial membranes. Accordingly it is desirable to select one or more penetration enhancers that facilitate the uptake of an oligonucleotide, without interfering with the activity of the oligonucleotide, and in a such a manner the oligonucleotide can be introduced into the body of an animal without unacceptable side-effects such as toxicity, irritation or allergic response.

Embodiments of the present invention provide compositions comprising one or more pharmaceutically acceptable penetration enhancers, and methods of using such compositions, which result in the improved bioavailability of oligonucleotides administered via non-parenteral modes of administration. Heretofore, certain penetration enhancers have been used to improve the bioavailability of certain drugs. See Muranishi, Crit. Rev. Ther. Drug Carrier Systems, 1990, 7, 1 and Lee et al., Crit. Rev. Ther. Drug Carrier Systems, 1991, 8, 91. It has been found that the uptake and delivery of oligonucleotides, relatively complex molecules which are known to be difficult to administer to animals and man, can be greatly improved even when administered by non-parenteral means through the use of a number of different classes of penetration enhancers.

In some embodiments, compositions for non-parenteral administration include one or more modifications from naturally-occurring oligonucleotides (i.e. full-phosphodiester deoxyribosyl or full-phosphodiester ribosyl oligonucleotides). Such modifications may increase binding affinity, nuclease stability, cell or tissue permeability, tissue distribution, or other biological or pharmacokinetic property. Modifications may be made to the base, the linker, or the sugar, in general, as discussed in more detail herein with regards to oligonucleotide chemistry. In some embodiments of the invention, compositions for administration to a subject, and in particular oral compositions for administration to an animal or human subject, will comprise modified oligonucleotides having one or more modifications for enhancing affinity, stability, tissue distribution, or other biological property.

Suitable modified linkers include phosphorothioate linkers. In some embodiments according to the invention, the oligonucleotide has at least one phosphorothioate linker. Phosphorothioate linkers provide nuclease stability as well as plasma protein binding characteristics to the oligonucleotide. Nuclease stability is useful for increasing the in vivo lifetime of oligonucleotides, while plasma protein binding decreases the rate of first pass clearance of oligonucleotide via renal excretion. In some embodiments according to the present invention, the oligonucleotide has at least two phosphorothioate linkers. In some embodiments, wherein the oligonucleotide has exactly n nucleosides, the oligonucleotide has from one to n−1 phosphorothioate linkages. In some embodiments, wherein the oligonucleotide has exactly n nucleosides, the oligonucleotide has n−1 phosphorothioate linkages. In other embodiments wherein the oligonucleotide has exactly n nucleoside, and n is even, the oligonucleotide has from 1 to n/2 phosphorothioate linkages, or, when n is odd, from 1 to (n−1)/2 phosphorothioate linkages. In some embodiments, the oligonucleotide has alternating phosphodiester (PO) and phosphorothioate (PS) linkages. In other embodiments, the oligonucleotide has at least one stretch of two or more consecutive PO linkages and at least one stretch of two or more PS linkages. In other embodiments, the oligonucleotide has at least two stretches of PO linkages interrupted by at least on PS linkage.

In some embodiments, at least one of the nucleosides is modified on the ribosyl sugar unit by a modification that imparts nuclease stability, binding affinity or some other beneficial biological property to the sugar. In some cases, the sugar modification includes a 2′-modification, e.g. the 2′-OH of the ribosyl sugar is replaced or substituted. Suitable replacements for 2′-OH include 2′-F and 2′-arabino-F. Suitable substitutions for OH include 2′-O-alkyl, e.g. 2-O-methyl, and 2′-O-substituted alkyl, e.g. 2′-O-methoxyethyl, 2′-O-aminopropyl, etc. In some embodiments, the oligonucleotide contains at least one 2′-modification. In some embodiments, the oligonucleotide contains at least 2 2′-modifications. In some embodiments, the oligonucleotide has at least one 2′-modification at each of the termini (i.e. the 3′- and 5′-terminal nucleosides each have the same or different 2′-modifications). In some embodiments, the oligonucleotide has at least two sequential 2′-modifications at each end of the oligonucleotide. In some embodiments, oligonucleotides further comprise at least one deoxynucleoside. In particular embodiments, oligonucleotides comprise a stretch of deoxynucleosides such that the stretch is capable of activating RNase (e.g. RNase H) cleavage of an RNA to which the oligonucleotide is capable of hybridizing. In some embodiments, a stretch of deoxynucleosides capable of activating RNase-mediated cleavage of RNA comprises about 6 to about 16, e.g. about 8 to about 16 consecutive deoxynucleosides. In further embodiments, oligonucleotides are capable of eliciting cleaveage by dsRNAse enzymes.

Oral compositions for administration of non-parenteral oligonucleotide compositions of the present invention may be formulated in various dosage forms such as, but not limited to, tablets, capsules, liquid syrups, soft gels, suppositories, and enemas. The term “alimentary delivery” encompasses e.g. oral, rectal, endoscopic and sublingual/buccal administration. A common requirement for these modes of administration is absorption over some portion or all of the alimentary tract and a need for efficient mucosal penetration of the nucleic acid(s) so administered.

Delivery of a drug via the oral mucosa, as in the case of buccal and sublingual administration, has several desirable features, including, in many instances, a more rapid rise in plasma concentration of the drug than via oral delivery (Harvey, Chapter 35 In: Remington's Pharmaceutical Sciences, 18th Ed., Gennaro, ed., Mack Publishing Co., Easton, Pa., 1990, page 711).

Endoscopy may be used for drug delivery directly to an interior portion of the alimentary tract. For example, endoscopic retrograde cystopancreatography (ERCP) takes advantage of extended gastroscopy and permits selective access to the biliary tract and the pancreatic duct (Hirahata et al., Gan To Kagaku Ryoho, 1992, 19(10 Suppl.), 1591). Pharmaceutical compositions, including liposomal formulations, can be delivered directly into portions of the alimentary canal, such as, e.g., the duodenum (Somogyi et al., Pharm. Res., 1995, 12, 149) or the gastric submucosa (Akamo et al., Japanese J. Cancer Res., 1994, 85, 652) via endoscopic means. Gastric lavage devices (Inoue et al., Artif. Organs, 1997, 21, 28) and percutaneous endoscopic feeding devices (Pennington et al., Ailment Pharmacol. Ther., 1995, 9, 471) can also be used for direct alimentary delivery of pharmaceutical compositions.

In some embodiments, oligonucleotide formulations may be administered through the anus into the rectum or lower intestine. Rectal suppositories, retention enemas or rectal catheters can be used for this purpose and may be preferred when patient compliance might otherwise be difficult to achieve (e.g., in pediatric and geriatric applications, or when the patient is vomiting or unconscious). Rectal administration can result in more prompt and higher blood levels than the oral route. (Harvey, Chapter 35 In: Remington's Pharmaceutical Sciences, 18th Ed., Gennaro, ed., Mack Publishing Co., Easton, Pa., 1990, page 711). Because about 50% of the drug that is absorbed from the rectum will bypass the liver, administration by this route significantly reduces the potential for first-pass metabolism (Benet et al., Chapter 1 In: Goodman & Gilman's The Pharmacological Basis of Therapeutics, 9th Ed., Hardman et al., eds., McGraw-Hill, New York, N.Y., 1996).

One advantageous method of non-parenteral administration oligonucleotide compositions is oral delivery. Some embodiments employ various penetration enhancers in order to effect transport of oligonucleotides and other nucleic acids across mucosal and epithelial membranes. Penetration enhancers may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Accordingly, some embodiments comprise oral oligonucleotide compositions comprising at least one member of the group consisting of surfactants, fatty acids, bile salts, chelating agents, and non-chelating surfactants. Further embodiments comprise oral oligonucleotide comprising at least one fatty acid, e.g. capric or lauric acid, or combinations or salts thereof. Other embodiments comprise methods of enhancing the oral bioavailability of an oligonucleotide, the method comprising co-administering the oligonucleotide and at least one penetration enhancer.

Other excipients that may be added to oral oligonucleotide compositions include surfactants (or “surface-active agents”), which are chemical entities which, when dissolved in an aqueous solution, reduce the surface tension of the solution or the interfacial tension between the aqueous solution and another liquid, with the result that absorption of oligonucleotides through the alimentary mucosa and other epithelial membranes is enhanced. In addition to bile salts and fatty acids, surfactants include, for example, sodium lauryl sulfate, polyoxyethylene-9-lauryl ether and polyoxyethylene-20-cetyl ether (Lee et al., Critical Reviews in Therapeutic Drug Carrier Systems, 1991, page 92); and perfluorohemical emulsions, such as FC-43 (Takahashi et al., J. Pharm. Phamacol., 1988, 40, 252).

Fatty acids and their derivatives which act as penetration enhancers and may be used in compositions of the present invention include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glyceryl 1-monocaprate, 1-dodecylazacycloheptan-2-one, acylcarnitines, acylcholines and mono- and di-glycerides thereof and/or physiologically acceptable salts thereof (i.e., oleate, laurate, caprate, myristate, palmitate, stearate, linoleate, etc.) (Lee et al., Critical Reviews in Therapeutic Drug Carrier Systems, 1991, page 92; Muranishi, Critical Reviews in Therapeutic Drug Carrier Systems, 1990, 7, 1; El-Hariri et al., J. Pharm. Pharmacol., 1992, 44, 651).

In some embodiments, oligonucleotide compositions for oral delivery comprise at least two discrete phases, which phases may comprise particles, capsules, gel-capsules, microspheres, etc. Each phase may contain one or more oligonucleotides, penetration enhancers, surfactants, bioadhesives, effervescent agents, or other adjuvant, excipient or diluent. In some embodiments, one phase comprises at least one oligonucleotide and at least one penetration enhancer. In some embodiments, a first phase comprises at least one oligonucleotide and at least one penetration enhancer, while a second phase comprises at least one penetration enhancer. In some embodiments, a first phase comprises at least one oligonucleotide and at least one penetration enhancer, while a second phase comprises at least one penetration enhancer and substantially no oligonucleotide. In some embodiments, at least one phase is compounded with at least one degradation retardant, such as a coating or a matrix, which delays release of the contents of that phase. In some embodiments, a first phase comprises at least one oligonucleotide, at least one penetration enhancer, while a second phase comprises at least one penetration enhancer and a release-retardant. In particular embodiments, an oral oligonucleotide comprises a first phase comprising particles containing an oligonucleotide and a penetration enhancer, and a second phase comprising particles coated with a release-retarding agent and containing penetration enhancer.

A variety of bile salts also function as penetration enhancers to facilitate the uptake and bioavailability of drugs. The physiological roles of bile include the facilitation of dispersion and absorption of lipids and fat-soluble vitamins (Brunton, Chapter 38 In: Goodman & Gilman's The Pharmacological Basis of Therapeutics, 9th Ed., Hardman et al., eds., McGraw-Hill, New York, N.Y., 1996, pages 934-935). Various natural bile salts, and their synthetic derivatives, act as penetration enhancers. Thus, the term “bile salt” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (CDCA, sodium chenodeoxycholate), ursodeoxycholic acid (UDCA), sodium tauro-24,25-dihydrofusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1; Yamamoto et al., J. Pharm. Exp. Ther., 1992, 263, 25; Yamashita et al., J. Pharm. Sci., 1990, 79, 579).

In some embodiments, penetration enhancers useful in some embodiments of present invention are mixtures of penetration enhancing compounds. One such penetration enhancer is a mixture of UDCA (and/or CDCA) with capric and/or lauric acids or salts thereof e.g. sodium. Such mixtures are useful for enhancing the delivery of biologically active substances across mucosal membranes, in particular intestinal mucosa. Other penetration enhancer mixtures comprise about 5-95% of bile acid or salt(s) UDCA and/or CDCA with 5-95% capric and/or lauric acid. Particular penetration enhancers are mixtures of the sodium salts of UDCA, capric acid and lauric acid in a ratio of about 1:2:2 respectively. Anther such penetration enhancer is a mixture of capric and lauric acid (or salts thereof) in a 0.01:1 to 1:0.01 ratio (mole basis). In particular embodiments capric acid and lauric acid are present in molar ratios of e.g. about 0.1:1 to about 1:0.1, in particular about 0.5:1 to about 1:0.5.

Other excipients include chelating agents, i.e. compounds that remove metallic ions from solution by forming complexes therewith, with the result that absorption of oligonucelotides through the alimentary and other mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315). Chelating agents of the invention include, but are not limited to, disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems, 1991, page 92; Muranishi, Critical Reviews in Therapeutic Drug Carrier Systems, 1990, 7, 1; Buur et al., J. Control Rel., 1990, 14, 43).

As used herein, non-chelating non-surfactant penetration enhancers may be defined as compounds that demonstrate insignificant activity as chelating agents or as surfactants but that nonetheless enhance absorption of oligonucleotides through the alimentary and other mucosal membranes (Muranishi, Critical Reviews in Therapeutic Drug Carrier Systems, 1990, 7, 1). This class of penetration enhancers includes, but is not limited to, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621).

Agents that enhance uptake of oligonucleotides at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), can be used.

Some oral oligonucleotide compositions also incorporate carrier compounds in the formulation. As used herein, “carrier compound” or “carrier” can refer to a nucleic acid, or analog thereof, which may be inert (i.e., does not possess biological activity per se) or may be necessary for transport, recognition or pathway activation or mediation, or is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5, 115; Takakura et al., Antisense & Nucl. Acid Drug Dev., 1996, 6, 177).

A “pharmaceutical carrier” or “excipient” may be a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinised maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, EXPLOTAB); and wetting agents (e.g., sodium lauryl sulphate, etc.).

Oral oligonucleotide compositions may additionally contain other adjunct components conventionally found in pharmaceutical compositions, at their art-established usage levels. Thus, for example, the compositions may contain additional, compatible, pharmaceutically-active materials such as, for example, antipuritics, astringents, local anesthetics or anti-inflammatory agents, or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions of the present invention.

Certain embodiments of the invention provide pharmaceutical compositions containing one or more oligomeric compounds and one or more other chemotherapeutic agents which function by a non-antisense mechanism. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Combinations of antisense compounds and other non-antisense drugs are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Alternatively, compositions of the invention may contain two or more antisense compounds targeted to different regions of the same nucleic acid target. Numerous examples of antisense compounds are known in the art. Two or more combined compounds may be used together or sequentially.

H. Dosing

The formulation of therapeutic compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 μg to 100 g per kg of body weight, from 0.1 μg to 10 g per kg of body weight, from 1.0 μg to 1 g per kg of body weight, from 10.0 μg to 100 mg per kg of body weight, from 100 μg to 10 mg per kg of body weight, or from 1 mg to 5 mg per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years.

The effects of treatments with therapeutic compositions can be assessed following collection of tissues or fluids from a patient or subject receiving said treatments. It is known in the art that a biopsy sample can be procured from certain tissues without resulting in detrimental effects to a patient or subject. In certain embodiments, a tissue and its constituent cells comprise, but are not limited to, blood (e.g., hematopoietic cells, such as human hematopoietic progenitor cells, human hematopoietic stem cells, CD34⁺ cells CD4⁺ cells), lymphocytes and other blood lineage cells, bone marrow, breast, cervix, colon, esophagus, lymph node, muscle, peripheral blood, oral mucosa and skin. In other embodiments, a fluid and its constituent cells comprise, but are not limited to, blood, urine, semen, synovial fluid, lymphatic fluid and cerebro-spinal fluid. Tissues or fluids procured from patients can be evaluated for expression levels of the target mRNA or protein. Additionally, the mRNA or protein expression levels of other genes known or suspected to be associated with the specific disease state, condition or phenotype can be assessed. mRNA levels can be measured or evaluated by real-time PCR, Northern blot, in situ hybridization or DNA array analysis. Protein levels can be measured or evaluated by ELISA, immunoblotting, quantitative protein assays, protein activity assays (for example, caspase activity assays) immunohistochemistry or immunocytochemistry. Furthermore, the effects of treatment can be assessed by measuring biomarkers associated with the disease or condition in the aforementioned tissues and fluids, collected from a patient or subject receiving treatment, by routine clinical methods known in the art. These biomarkers include but are not limited to: glucose, cholesterol, lipoproteins, triglycerides, free fatty acids and other markers of glucose and lipid metabolism; liver transaminases, bilirubin, albumin, blood urea nitrogen, creatine and other markers of kidney and liver function; interleukins, tumor necrosis factors, intracellular adhesion molecules, C-reactive protein and other markers of inflammation; testosterone, estrogen and other hormones; tumor markers; vitamins, minerals and electrolytes.

While the present invention has been described with specificity in accordance with certain of its preferred embodiments, the following examples serve only to illustrate the invention and are not intended to limit the same. Each of the references, GenBank accession numbers, and the like recited in the present application is incorporated herein by reference in its entirety.

### EXAMPLES

### Example 1

Synthesis of Nucleoside Phosphoramidites

The following compounds, including amidites and their intermediates were prepared as described in U.S. Pat. No. 6,426,220 and published PCT WO 02/36743; 5′-O-Dimethoxytrityl-thymidine intermediate for 5-methyl dC amidite, 5′-O-Dimethoxytrityl-2′-deoxy-5-methylcytidine intermediate for 5-methyl-dC amidite, 5′-O-Dimethoxytrityl-2′-deoxy-N-4-benzoyl-5-methylcytidine penultimate intermediate for 5-methyl dC amidite, [5′-O-(4,4′-Dimethoxytriphenylmethyl)-2′-deoxy-N⁴-benzoyl-5-methylcytidin-3′-O-yl]-2-cyanoethyl-N,N-diisopropylphosphoramidite (5-methyl dC amidite), 2′-Fluorodeoxyadenosine, 2′-Fluorodeoxyguanosine, 2′-Fluorouridine, 2′-Fluorodeoxycytidine, 2′-O-(2-Methoxyethyl) modified amidites, 2′-O-(2-methoxyethyl)-5-methyluridine intermediate, 5′-O-DMT-2′-O-(2-methoxyethyl)-5-methyluridine penultimate intermediate, [5′-O-(4,4′-Dimethoxytriphenylmethyl)-2′-O-(2-methoxyethyl)-5-methyluridin-3′-O-yl]-2-cyanoethyl-N,N-diisopropylphosphoramidite (MOE T amidite), 51-O-Dimethoxytrityl-2′-O-(2-methoxyethyl)-5-methylcytidine intermediate, 5′-O-dimethoxytrityl-2′-O-(2-methoxyethyl)-N⁴-benzoyl-5-methyl-cytidine penultimate intermediate, [5′-O-(4,4′-Dimethoxytriphenylmethyl)-2′-O-(2-methoxyethyl)-N-benzoyl-5-methylcytidin-3′-O-yl]-2-cyanoethyl-N,N-diisopropylphosphoramidite (MOE 5-Me-C amidite), [5′-O-(4,4′-Dimethoxytriphenylmethyl)-21′-O-(2-methoxyethyl)-N⁶-benzoyladenosin-3′-O-yl]-2-cyanoethyl-N,N-diisopropylphosphoramidite (MOE A amdite), [5′-O-(4,4′-Dimethoxytriphenylmethyl)-2′-O-(2-methoxyethyl)-N-isobutyrylguanosin-3′-O-yl]-2-cyanoethyl-N,N-diisopropylphosphoramidite (MOE G amidite), 2′-O-(Aminooxyethyl) nucleoside amidites and 2′-O-(dimethylaminooxyethyl) nucleoside amidites, 2′-(Dimethylaminooxyethoxy) nucleoside amidites, 5′-O-tert-Butyldiphenylsilyl-O²-2′-anhydro-5-methyluridine, 5′-O-tert-Butyldiphenylsilyl-2′-O-(2-hydroxyethyl)-5-methyluridine, 2′-O-([2-phthalimidoxy)ethyl]-5′-t-butyldiphenylsilyl-5-methyluridine 5′-O-tert-butyldiphenylsilyl-2′-O-[(2-formadoximinooxy)ethyl]-5-methyluridine, 5′-O-tert-Butyldiphenylsilyl-2′-O-[N,N dimethylaminooxyethyl]-5-methyluridine, 2′-O-(dimethylaminooxyethyl)-5-methyluridine, 5′-O-DMT-2′-O-(dimethylaminooxyethyl)-5-methyluridine, 5′-O-DMT-2′-O-(2-N,N-dimethylaminooxyethyl)-5-methyluridine-3′-[(2-cyanoethyl)-N,N-diisopropylphosphoramidite], 2′-(Aminooxyethoxy) nucleoside amidites, N2-isobutyryl-6-O-diphenylcarbamoyl-2′-O-(2-ethylacetyl)-5′-O-(4,4′-30 dimethoxytrityl)guanosine-3′-[(2-cyanoethyl)-N,N-diisopropylphosphoramidite], 2′-dimethylaminoethoxyethoxy (2′-DMAEOE) nucleoside amidites, 2-O-[2(2-N,N-dimethylaminoethoxy)ethyl]-5-methyl uridine, 5′-O-dimethoxytrityl-2′-O-[2(2-N,N-dimethylaminoethoxy)-ethyl)]-5-methyl uridine and 5′-O-Dimethoxytrityl-2′-O-[2(2-N,N-dimethylaminoethoxy)-ethyl)]-5-methyl uridine-3′-O-(cyanoethyl-N,N-diisopropyl)phosphoramidite.

### Example 2

Oligonucleotide and Oligonucleoside Synthesis

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Oligonucleotides: Unsubstituted and substituted phosphodiester (P═O) oligonucleotides are synthesized on an automated DNA synthesizer (Applied Biosystems model 394) using standard phosphoramidite chemistry with oxidation by iodine.

Phosphorothioates (P═S) are synthesized similar to phosphodiester oligonucleotides with the following exceptions: thiation was effected by utilizing a 10% w/v solution of 3,H-1,2-benzodithiole-3-one 1,1-dioxide in acetonitrile for the oxidation of the phosphite linkages. The thiation reaction step time was increased to 180 sec and preceded by the normal capping step. After cleavage from the CPG column and deblocking in concentrated ammonium hydroxide at 55° C. (12-16 hr), the oligonucleotides were recovered by precipitating with >3 volumes of ethanol from a 1 M NH₄OAc solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. No. 5,610,289 or 5,625,050, herein incorporated by reference.

Phosphoramidite oligonucleotides are prepared as described in U.S. Pat. No. 5,256,775 or U.S. Pat. No. 5,366,878, herein incorporated by reference.

Alkylphosphonothioate oligonucleotides are prepared as described in published PCT applications PCT/US94/00902 and PCT/US93/06976 (published as WO 94/17093 and WO 94/02499, respectively), herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

Oligonucleosides: Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### Example 3

RNA Synthesis

In general, RNA synthesis chemistry is based on the selective incorporation of various protecting groups at strategic intermediary reactions. Although one of ordinary skill in the art will understand the use of protecting groups in organic synthesis, a useful class of protecting groups includes silyl ethers. In particular bulky silyl ethers are used to protect the 5′-hydroxyl in combination with an acid-labile orthoester protecting group on the 2′-hydroxyl. This set of protecting groups is then used with standard solid-phase synthesis technology. It is important to lastly remove the acid labile orthoester protecting group after all other synthetic steps. Moreover, the early use of the silyl protecting groups during synthesis ensures facile removal when desired, without undesired deprotection of 2′ hydroxyl.

Following this procedure for the sequential protection of the 5′-hydroxyl in combination with protection of the 2′-hydroxyl by protecting groups that are differentially removed and are differentially chemically labile, RNA oligonucleotides were synthesized.

RNA oligonucleotides are synthesized in a stepwise fashion. Each nucleotide is added sequentially (3′- to 5′-direction) to a solid support-bound oligonucleotide. The first nucleoside at the 3′-end of the chain is covalently attached to a solid support. The nucleotide precursor, a ribonucleoside phosphoramidite, and activator are added, coupling the second base onto the 5′-end of the first nucleoside. The support is washed and any unreacted 5′-hydroxyl groups are capped with acetic anhydride to yield 5′-acetyl moieties. The linkage is then oxidized to the more stable and ultimately desired P(V) linkage. At the end of the nucleotide addition cycle, the 5′-silyl group is cleaved with fluoride. The cycle is repeated for each subsequent nucleotide.

Following synthesis, the methyl protecting groups on the phosphates are cleaved in 30 minutes utilizing 1 M disodium-2-carbamoyl-2-cyanoethylene-1,1-dithiolate trihydrate (S₂Na₂) in DMF. The deprotection solution is washed from the solid support-bound oligonucleotide using water. The support is then treated with 40% methylamine in water for 10 minutes at 55° C. This releases the RNA oligonucleotides into solution, deprotects the exocyclic amines, and modifies the 2′-groups. The oligonucleotides can be analyzed by anion exchange HPLC at this stage.

The 2′-orthoester groups are the last protecting groups to be removed. The ethylene glycol monoacetate orthoester protecting group developed by Dharmacon Research, Inc. (Lafayette, Colo.), is one example of a useful orthoester protecting group which, has the following important properties. It is stable to the conditions of nucleoside phosphoramidite synthesis and oligonucleotide synthesis. However, after oligonucleotide synthesis the oligonucleotide is treated with methylamine which not only cleaves the oligonucleotide from the solid support but also removes the acetyl groups from the orthoesters. The resulting 2-ethylhydroxyl substituents on the orthoester are less electron withdrawing than the acetylated precursor. As a result, the modified orthoester becomes more labile to acid-catalyzed hydrolysis. Specifically, the rate of cleavage is approximately 10 times faster after the acetyl groups are removed. Therefore, this orthoester possesses sufficient stability in order to be compatible with oligonucleotide synthesis and yet, when subsequently modified, permits deprotection to be carried out under relatively mild aqueous conditions compatible with the final RNA oligonucleotide product.

Additionally, methods of RNA synthesis are well known in the art (Scaringe, S. A. Ph.D. Thesis, University of Colorado, 1996; Scaringe, S. A., et al., J. Am. Chem. Soc., 1998, 120, 11820-11821; Matteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862; Dahl, B. J., et al., Acta Chem. Scand., 1990, 44, 639-641; Reddy, M. P., et al., Tetrahedrom Lett., 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331).

RNA antisense compounds (RNA oligonucleotides) of the present invention can be synthesized by the methods herein or purchased from Dharmacon Research, Inc (Lafayette, Colo.). Once synthesized, complementary RNA antisense compounds can then be annealed by methods known in the art to form double stranded (duplexed) antisense compounds. For example, duplexes can be formed by combining 30 μl of each of the complementary strands of RNA oligonucleotides (50 uM RNA oligonucleotide solution) and 15 μl of 5× annealing buffer (100 mM potassium acetate, 30 mM HEPES-KOH pH 7.4, 2 mM magnesium acetate) followed by heating for 1 minute at 90° C., then 1 hour at 37° C. The resulting duplexed antisense compounds can be used in kits, assays, screens, or other methods to investigate the role of a target nucleic acid, or for diagnostic or therapeutic purposes.

### Example 4

Synthesis of Chimeric Compounds

Chimeric oligonucleotides, oligonucleosides or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

### [2′-O-Me]--[2′-deoxy]--[2′-O-Me] Chimeric Phosphorothioate Oligonucleotides

Chimeric oligonucleotides having 2′-O-alkyl phosphorothioate and 2′-deoxy phosphorothioate oligonucleotide segments are synthesized using an Applied Biosystems automated DNA synthesizer Model 394, as above. Oligonucleotides are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by incorporating coupling steps with increased reaction times for the 5′-dimethoxytrityl-2′-O-methyl-3′-O-phosphoramidite. The fully protected oligonucleotide is cleaved from the support and deprotected in concentrated ammonia (NH₄OH) for 12-16 hr at 55° C. The deprotected oligo is then recovered by an appropriate method (precipitation, column chromatography, volume reduced in vacuo and analyzed spetrophotometrically for yield and for purity by capillary electrophoresis and by mass spectrometry.

### [2′-O-(2-Methoxyethyl)]--[2′-deoxy]--[2′-O-(Methoxyethyl)]Chimeric Phosphorothioate Oligonucleotides

[2′-O-(2-methoxyethyl)]--[2′-deoxy]--[-2′-O-(methoxyethyl)]chimeric phosphorothioate oligonucleotides were prepared as per the procedure above for the 2′-O-methyl chimeric oligonucleotide, with the substitution of 2′-O-(methoxyethyl) amidites for the 2′-O-methyl amidites.

### [2′-O-(2-Methoxyethyl)Phosphodiester]--[2′-deoxy Phosphorothioate]--[2′-O-(2-Methoxyethyl) Phosphodiester]Chimeric Oligonucleotides

[2′-O-(2-methoxyethyl phosphodiester]--[2′-deoxy phosphorothioate]--[2′-O-(methoxyethyl) phosphodiester]chimeric oligonucleotides are prepared as per the above procedure for the 2′-O-methyl chimeric oligonucleotide with the substitution of 2′-O-(methoxyethyl) amidites for the 2′-O-methyl amidites, oxidation with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides and mixed chimeric oligonucleotides/oligonucleosides are synthesized according to U.S. Pat. No. 5,623,065, herein incorporated by reference.

### Example 5

Design and Screening of Duplexed Antisense Compounds Targeting Notch2

In accordance with the present invention, a series of nucleic acid duplexes comprising the antisense compounds of the present invention and their complements can be designed to target Notch2. The nucleobase sequence of the antisense strand of the duplex comprises at least an 8-nucleobase portion of an oligonucleotide in Table 1. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini. The antisense and sense strands of the duplex comprise from about 17 to 25 nucleotides, or from about 19 to 23 nucleotides. Alternatively, the antisense and sense strands comprise 20, 21 or 22 nucleotides.

For example, a duplex comprising an antisense strand having the sequence CGAGAGGCGGACGGGACCG and having a two-nucleobase overhang of deoxythymidine(dT) would have the following structure:

| | |
|---|---|
| cgagaggcggacgggaccgTT | Antisense Strand |
| \|\|\|\|\|\|\|\|\|\|\|\|\|\|\|\|\|\|\| | |
| TTgctctccgcctgccctggc | Complement |

Overhangs can range from 2 to 6 nucleobases and these nucleobases may or may not be complementary to the target nucleic acid. In another embodiment, the duplexes may have an overhang on only one terminus.

In another embodiment, a duplex comprising an antisense strand having the same sequence CGAGAGGCGGACGGGACCG may be prepared with blunt ends (no single stranded overhang) as shown:

| | |
|---|---|
| cgagaggcggacgggaccg | Antisense Strand |
| \|\|\|\|\|\|\|\|\|\|\|\|\|\|\|\|\|\|\| | |
| gctctccgcctgccctggc | Complement |

The RNA duplex can be unimolecular or bimolecular; i.e, the two strands can be part of a single molecule or may be separate molecules.

RNA strands of the duplex can be synthesized by methods disclosed herein or purchased from Dharmacon Research Inc., (Lafayette, Colo.). Once synthesized, the complementary strands are annealed. The single strands are aliquoted and diluted to a concentration of 50 uM. Once diluted, 30 uL of each strand is combined with 15 uL of a 5× solution of annealing buffer. The final concentration of said buffer is 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 uM. This solution can be stored frozen (−20° C.) and freeze-thawed up to 5 times.

Once prepared, the duplexed antisense compounds are evaluated for their ability to modulate Notch2 expression.

When cells reached 80% confluency, they are treated with duplexed antisense compounds of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN (Gibco BRL) and the desired duplex antisense compound at a final concentration of 200 nM. After 5 hours of treatment, the medium is replaced with fresh medium. Cells are harvested 16 hours after treatment, at which time RNA is isolated and target reduction measured by RT-PCR.

### Example 6

Oligonucleotide Isolation

After cleavage from the controlled pore glass solid support and deblocking in concentrated ammonium hydroxide at 55° C. for 12-16 hours, the oligonucleotides or oligonucleosides are recovered by precipitation out of 1 M NH₄OAc with >3 volumes of ethanol. Synthesized oligonucleotides were analyzed by electrospray mass spectroscopy (molecular weight determination) and by capillary gel electrophoresis and judged to be at least 70% full length material. The relative amounts of phosphorothioate and phosphodiester linkages obtained in the synthesis was determined by the ratio of correct molecular weight relative to the −16 amu product (+/−32+/−48). For some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 7

Oligonucleotide Synthesis—96 Well Plate Format

Oligonucleotides were synthesized via solid phase P(III) phosphoramidite chemistry on an automated synthesizer capable of assembling 96 sequences simultaneously in a 96-well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites were purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites.

Oligonucleotides were cleaved from support and deprotected with concentrated NH₄OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### Example 8

Oligonucleotide Analysis—96-Well Plate Format

The concentration of oligonucleotide in each well was assessed by dilution of samples and UV absorption spectroscopy. The full-length integrity of the individual products was evaluated by capillary electrophoresis (CE) in either the 96-well format (Beckman P/ACE™ MDQ) or, for individually prepared samples, on a commercial CE apparatus (e.g., Beckman P/ACE™ 5000, ABI 270). Base and backbone composition was confirmed by mass analysis of the compounds utilizing electrospray-mass spectroscopy. All assay test plates were diluted from the master plate using single and multi-channel robotic pipettors. Plates were judged to be acceptable if at least 85% of the compounds on the plate were at least 85% full length.

### Example 9

Cell Culture and Oligonucleotide Treatment

The effect of antisense compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, ribonuclease protection assays, or RT-PCR.

T-24 Cells:

The human transitional cell bladder carcinoma cell line T-24 was obtained from the American Type Culture Collection (ATCC) (Manassas, Va.). T-24 cells were routinely cultured in complete McCoy's 5A basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence. Cells were seeded into 96-well plates (Falcon-Primaria #353872) at a density of 7000 cells/well for use in RT-PCR analysis.

For Northern blotting or other analysis, cells may be seeded onto 100 mm or other standard tissue culture plates and treated similarly, using appropriate volumes of medium and oligonucleotide.

A549 Cells:

The human lung carcinoma cell line A549 was obtained from the American Type Culture Collection (ATCC) (Manassas, Va.). A549 cells were routinely cultured in DMEM basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence.

NHDF Cells:

Human neonatal dermal fibroblast (NHDF) were obtained from the Clonetics Corporation (Walkersville, Md.). NHDFs were routinely maintained in Fibroblast Growth Medium (Clonetics Corporation, Walkersville, Md.) supplemented as recommended by the supplier. Cells were maintained for up to 10 passages as recommended by the supplier.

HEK Cells:

Human embryonic keratinocytes (HEK) were obtained from the Clonetics Corporation (Walkersville, Md.). HEKs were routinely maintained in Keratinocyte Growth Medium (Clonetics Corporation, Walkersville, Md.) formulated as recommended by the supplier. Cells were routinely maintained for up to 10 passages as recommended by the supplier.

Treatment with Antisense Compounds:

When cells reached 65-75% confluency, they were treated with oligonucleotide. Oligonucleotides were mixed with LIPOFECTIN™ (Invitrogen Life Technologies, Carlsbad, Calif.) in 1 mL of Opti-MEM™-1 reduced serum medium (Invitrogen Life Technologies, Carlsbad, Calif.) to achieve the desired concentration of oligonucleotide. This transfection mixture was incubated at room temperature for approximately 0.5 hours. LIPOFECTIN™ is used at a concentration of 2.5 or 3 μg/mL LIPOFECTIN™ per 100 nM oligonucleotide. For cells grown in 96-well plates, wells were washed once with 100 μL OPTI-MEM™-1 and then treated with 130 μL of the transfection mixutre. Cells grown in 24-well plates or other standard tissue culture plates are treated similarly, using appropriate volumes of medium and oligonucleotide. Cells are treated and data are obtained in duplicate or triplicate. After approximately 4-7 hours of treatment at 37° C., the medium containing the transfection mixture was replaced with fresh medium. Cells were harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is selected from either ISIS 13920 (TCCGTCATCGCTCCTCAGGG, SEQ ID NO: 1) which is targeted to human H-ras, or ISIS 18078, (GTGCGCGCGAGCCCGAAATC, SEQ ID NO: 2) which is targeted to human Jun-N-terminal kinase-2 (JNK2). Both controls are 2′-O-methoxyethyl gapmers (2′-O-methoxyethyls shown in bold) with a phosphorothioate backbone. For mouse or rat cells the positive control oligonucleotide is ISIS 15770, ATGCATTCTGCCCCCAAGGA, SEQ ID NO: 3, a 2′-O-methoxyethyl gapmer (2′-O-methoxyethyls shown in bold) with a phosphorothioate backbone which is targeted to both mouse and rat c-raf. The concentration of positive control oligonucleotide that results in 80% inhibition of c-H-ras (for ISIS 13920), JNK2 (for ISIS 18078) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of c-H-ras, JNK2 or c-raf mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 50 nM to 300 nM.

### Example 10

Analysis of Oligonucleotide Inhibition of Notch2 Expression

Antisense modulation of Notch2 expression can be assayed in a variety of ways known in the art. For example, Notch2 mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. The preferred method of RNA analysis of the present invention is the use of total cellular RNA as described in other examples herein. Methods of RNA isolation are well known in the art. Northern blot analysis is also routine in the art. Real-time quantitative (PCR) can be conveniently accomplished using the commercially available ABI PRISM™ 7600, 7700, or 7900 Sequence Detection System, available from PE-Applied Biosystems, Foster City, Calif. and used according to manufacturer's instructions.

Protein levels of Notch2 can be quantitated in a variety of ways well known in the art, such as immunoprecipitation, Western blot analysis (immunoblotting), enzyme-linked immunosorbent assay (ELISA) or fluorescence-activated cell sorting (FACS). Antibodies directed to Notch2 can be identified and obtained from a variety of sources, such as the MSRS catalog of antibodies (Aerie Corporation, Birmingham, Mich.), or can be prepared via conventional monoclonal or polyclonal antibody generation methods well known in the art.

### Example 11

Design of Phenotypic Assays for the Use of Notch2 Inhibitors

Phenotypic Assays

Once Notch2 inhibitors have been identified by the methods disclosed herein, the compounds are further investigated in one or more phenotypic assays, each having measurable endpoints predictive of efficacy in the treatment of a particular disease state or condition. Phenotypic assays, kits and reagents for their use are well known to those skilled in the art and are herein used to investigate the role and/or association of Notch2 in health and disease. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.)

In one non-limiting example, cells determined to be appropriate for a particular phenotypic assay (i.e., MCF-7 cells selected for breast cancer studies; adipocytes for obesity studies) are treated with Notch2 inhibitors identified from the in vitro studies as well as control compounds at optimal concentrations which are determined by the methods described above. At the end of the treatment period, treated and untreated cells are analyzed by one or more methods specific for the assay to determine phenotypic outcomes and endpoints.

Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Analysis of the genotype of the cell (measurement of the expression of one or more of the genes of the cell) after treatment is also used as an indicator of the efficacy or potency of the Notch2 inhibitors. Hallmark genes, or those genes suspected to be associated with a specific disease state, condition, or phenotype, are measured in both treated and untreated cells.

### Example 12

RNA Isolation

Poly(A)+ mRNA Isolation

Poly(A)+ mRNA was isolated according to Miura et al., (Clin. Chem., 1996, 42, 1758-1764). Other methods for poly(A)+ mRNA isolation are routine in the art. Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C., was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

Total RNA Isolation

Total RNA was isolated using an RNEASY 96™ kit and buffers purchased from Qiagen Inc. (Valencia, Calif.) following the manufacturer's recommended procedures. Briefly, for cells grown on 96-well plates, growth medium was removed from the cells and each well was washed with 200 μL cold PBS. 150 μL Buffer RLT was added to each well and the plate vigorously agitated for 20 seconds. 150 μL of 70% ethanol was then added to each well and the contents mixed by pipetting three times up and down. The samples were then transferred to the RNEASY 96™ well plate attached to a QIAVAC™ manifold fitted with a waste collection tray and attached to a vacuum source. Vacuum was applied for 1 minute. 500 μL of Buffer RW1 was added to each well of the RNEASY 96™ plate and incubated for 15 minutes and the vacuum was again applied for 1 minute. An additional 500 μL of Buffer RW1 was added to each well of the RNEASY 96™ plate and the vacuum was applied for 2 minutes. 1 mL of Buffer RPE was then added to each well of the RNEASY 96™ plate and the vacuum applied for a period of 90 seconds. The Buffer RPE wash was then repeated and the vacuum was applied for an additional 3 minutes. The plate was then removed from the QIAVAC™ manifold and blotted dry on paper towels. The plate was then re-attached to the QIAVAC™ manifold fitted with a collection tube rack containing 1.2 mL collection tubes. RNA was then eluted by pipetting 140 μL of RNAse free water into each well, incubating 1 minute, and then applying the vacuum for 3 minutes.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 13

Real-Time Quantitative PCR Analysis of Notch2 mRNA Levels

Quantitation of Notch2 mRNA levels was accomplished by real-time quantitative PCR using the ABI PRISM™ 7600, 7700, or 7900 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

Gene target quantities are obtained by real-time PCR. Prior to the real-time PCR, isolated RNA is subjected to a reverse transcriptase (RT) reaction, for the purpose of generating complementary DNA (cDNA). Reverse transcriptase and PCR reagents were obtained from Invitrogen Corporation, (Carlsbad, Calif.). RT, real-time PCR reactions were carried out by adding 20 μL PCR cocktail (2.5×PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension). The method of obtaining gene target quantities by RT, real-time PCR is herein referred to as real-time PCR.

Gene target quantities obtained by real-time PCR were normalized using either the expression level of GAPDH, a gene whose expression is constant, or by quantifying total RNA using RiboGreen™ (Molecular Probes, Inc. Eugene, Oreg.). GAPDH expression is quantified by real-time PCR by being run simultaneously with the target, multiplexing, or separately. Total RNA is quantified using RiboGreen™ RNA quantification reagent (Molecular Probes, Inc. Eugene, Oreg.). Methods of RNA quantification by RiboGreen™ are taught in Jones, L. J., et al, (Analytical Biochemistry, 1998, 265, 368-374).

In this assay, 170 μL of RiboGreen™ working reagent (RiboGreen™ reagent diluted 1:350 in 10 mM Tris-HCl, 1 mM EDTA, pH 7.5) was pipetted into a 96-well plate containing 30 μL purified, cellular RNA. The plate was read in a CytoFluor 4000 (PE Applied Biosystems) with excitation at 485 nm and emission at 530 nm.

Probes and primers to human Notch2 were designed to hybridize to a human Notch2 sequence, using published sequence information (a genomic sequence of human Notch2 is represented by the complement of residues 4894439-5015872 of GenBank accession number NT_{—}004754.7, incorporated herein as SEQ ID NO: 4). For human Notch2 the PCR primers were: forward primer: TGGCAACTAACGTAGAAACTCAACA (SEQ ID NO: 5) reverse primer: TGCCAAGAGCATGAATACAGAGA (SEQ ID NO: 6) and the PCR probe was: FAM-ACAACTATAGACTTGCTCATTGTTCAGACTGATTGCC-TAMRA (SEQ ID NO: 7) where FAM is the fluorescent dye and TAMRA is the quencher dye. For human GAPDH the PCR primers were: forward primer: GAAGGTGAAGGTCGGAGTC (SEQ ID NO:8) reverse primer: GAAGATGGTGATGGGATTTC (SEQ ID NO:9) and the PCR probe was: 5′ JOE-CAAGCTTCCCGTTCTCAGCC-TAMRA 3′ (SEQ ID NO: 10) where JOE is the fluorescent reporter dye and TAMRA is the quencher dye.

### Example 14

Northern Blot Analysis of Notch2 mRNA Levels

Eighteen hours after antisense treatment, cell monolayers were washed twice with cold PBS and lysed in 1 mL RNAZOL™ (TEL-TEST “B” Inc., Friendswood, Tex.). Total RNA was prepared following manufacturer's recommended protocols. Twenty micrograms of total RNA was fractionated by electrophoresis through 1.2% agarose gels containing 1.1% formaldehyde using a MOPS buffer system (AMRESCO, Inc. Solon, Ohio). RNA was transferred from the gel to HYBOND™-N+ nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer was confirmed by UV visualization. Membranes were fixed by UV cross-linking using a STRATALINKER™ UV Crosslinker 2400 (Stratagene, Inc, La Jolla, Calif.) and then probed using QUICKHYB™ hybridization solution (Stratagene, La Jolla, Calif.) using manufacturer's recommendations for stringent conditions.

To detect human Notch2, a human Notch2 specific probe was prepared by PCR using the forward primer TGGCAACTAACGTAGAAACTCAACA (SEQ ID NO: 5) and the reverse primer TGCCAAGAGCATGAATACAGAGA (SEQ ID NO: 6). To normalize for variations in loading and transfer efficiency membranes were stripped and probed for human glyceraldehyde-3-phosphate dehydrogenase (GAPDH) RNA (Clontech, Palo Alto, Calif.).

Hybridized membranes were visualized and quantitated using a PHOSPHORIMAGER™ and IMAGEQUANT™ Software V3.3 (Molecular Dynamics, Sunnyvale, Calif.). Data was normalized to GAPDH levels in untreated controls.

### Example 15

Antisense Inhibition of Human Notch2 Expression by Chimeric Phosphorothioate Oligonucleotides having 2′-MOE Wings and a Deoxy Gap

In accordance with the present invention, a series of antisense compounds was designed to target different regions of the human Notch2 RNA, using published sequences (a genomic sequence of human Notch2 represented by the complement of residues 4894439-5015872 of GenBank accession number NT_{—}004754.7, incorporated herein as SEQ ID NO: 4; GenBank accession number NM_{—}024408.1, incorporated herein as SEQ ID NO: 11; GenBank accession number BC010154.1 incorporated herein as SEQ ID NO: 12; and GenBank accession number BI562298.1, incorporated herein as SEQ ID NO: 13). The compounds are shown in Table 1. “Target site” indicates the first (5′-most) nucleotide number on the particular target sequence to which the compound binds. All compounds in Table 1 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds were analyzed for their effect on human Notch2 mRNA levels by quantitative real-time PCR as described in other examples herein. Data are averages from two experiments in which A549 cells were treated with antisense oligonucleotides of the present invention at 150 nM concentration. The negative control for each datapoint is identified in the table by sequence ID number. If present, “N.D.” indicates “no data”.

TABLE 1

Inhibition of human Notch2 mPNA levels by chimeric
phosphorothioate oligonucleotides having 2′-MOE wings
and a deoxy gap

| | | |
|---|---|---|
| TARGET | SEQ | CONTROL |

| | | | | | |
|---|---|---|---|---|---|
| SEQ ID | TARGET | SEQUENCE | % | ID | SEQ ID |

| | | | | | | | |
|---|---|---|---|---|---|---|---|
| ISIS # | REGION | NO | SITE | (5′ to 3′) | INHIB | NO | NO |

| | | | | | | | |
|---|---|---|---|---|---|---|---|
| 226896 | Coding | 4 | 25923 | tctcgatgttgacaatattc | 58 | 14 | 1 |
| 226897 | Coding | 11 | 418 | cactccttacctgtaaaccc | 24 | 15 | 1 |
| 226898 | Coding | 4 | 34233 | ggcatttgcaggagaactgg | 67 | 16 | 1 |
| 226899 | Coding | 4 | 44403 | gtgctcccttcaaaacctgg | 62 | 17 | 1 |
| 226900 | Coding | 4 | 63879 | tttcctgcatgctcacaagg | 79 | 18 | 1 |
| 226901 | Coding | 4 | 63923 | ccttcagacactcacagtgg | 76 | 19 | 1 |
| 226902 | Coding | 11 | 1455 | ctttgaaacctggcatgcac | 56 | 20 | 1 |
| 226903 | Coding | 4 | 64999 | caatgcacacctttgaaacc | 32 | 21 | 1 |
| 226904 | Coding | 4 | 65997 | tagccattcgggtgatcgat | 73 | 22 | 1 |
| 226905 | Coding | 4 | 66002 | attcatagccattcgggtga | 49 | 23 | 1 |
| 226906 | Coding | 4 | 66007 | ctggcattcatagccattcg | 61 | 24 | 1 |
| 226907 | Coding | 4 | 66013 | ggcacactggcattcatagc | 41 | 25 | 1 |
| 226908 | Coding | 4 | 66018 | cctgtggcacactggcattc | 53 | 26 | 1 |
| 226909 | Coding | 4 | 67768 | ttgcagatgcaggtgtagga | 69 | 27 | 1 |
| 226910 | Coding | 4 | 67813 | cattcatcaatctggtcact | 44 | 28 | 1 |
| 226911 | Coding | 4 | 71996 | gtcatcaaaattaatttcac | 50 | 29 | 1 |
| 226912 | Coding | 4 | 72001 | gcacagtcatcaaaattaat | 66 | 30 | 1 |
| 226913 | Coding | 4 | 76276 | tggaggcacactcatcaatg | 71 | 31 | 1 |
| 226914 | Coding | 4 | 76376 | ttcacctgtgagtagcagct | 76 | 32 | 1 |
| 226915 | Coding | 4 | 76424 | agacctccagtacagtttcc | 53 | 33 | 1 |
| 226916 | Coding | 4 | 76429 | cactgagacctccagtacag | 68 | 34 | 1 |
| 226917 | Coding | 4 | 80699 | aagcaggttccttggttcag | 72 | 35 | 1 |
| 226918 | Coding | 4 | 82461 | tgccagccaggagcacacaa | 68 | 36 | 1 |
| 226919 | Coding | 11 | 2755 | tggcaaggattggcaaggca | 73 | 37 | 1 |
| 226920 | Coding | 4 | 89815 | ttcatgtctgtctggcactt | 74 | 38 | 1 |
| 226921 | Coding | 11 | 3181 | agggtctgacagtttttccc | 62 | 39 | 1 |
| 226922 | Coding | 11 | 3186 | tcaccagggtctgacagttt | 65 | 40 | 1 |
| 226923 | Coding | 4 | 94030 | gctggcacaagtgttcaaca | 69 | 41 | 1 |
| 226924 | Coding | 4 | 95897 | tcacagttgacaccctgata | 80 | 42 | 1 |
| 226925 | Coding | 4 | 95902 | catactcacagttgacaccc | 30 | 43 | 1 |
| 226926 | Coding | 4 | 95907 | cacttcatactcacagttga | 59 | 44 | 1 |
| 226927 | Coding | 4 | 102378 | tccccagcaaagccaggcaa | 75 | 45 | 1 |
| 226928 | Cbding | 4 | 105700 | acttgtccacagctgctctg | 68 | 46 | 1 |
| 226929 | Coding | 4 | 106009 | ccatcccactggcaggcatg | 76 | 47 | 1 |
| 226930 | Coding | 4 | 106099 | tcatcacactggttgttgat | 77 | 48 | 1 |
| 226931 | Coding | 4 | 106135 | aagttgtcaaacaggcactc | 56 | 49 | 1 |
| 226932 | Coding | 11 | 4519 | cagtatttgtcatacttgca | 64 | 50 | 1 |
| 226933 | Coding | 4 | 107505 | ctgcacagtatttgtcatac | 80 | 51 | 1 |
| 226934 | Coding | 4 | 107510 | gtggtctgcacagtatttgt | 66 | 52 | 1 |
| 226935 | Coding | 4 | 107515 | ttgaagtggtctgcacagta | 69 | 53 | 1 |
| 226936 | Coding | 4 | 107520 | tgtctttgaagtggtctgca | 76 | 54 | 1 |
| 226937 | Coding | 4 | 107530 | tcacagtggttgtctttgaa | 61 | 55 | 1 |
| 226938 | Coding | 11 | 4862 | gactttagagccagccacct | 41 | 56 | 1 |
| 226939 | Coding | 4 | 111150 | agcttcaaggtgctgctgtg | 59 | 57 | 1 |
| 226940 | Coding | 4 | 111155 | tctgcagcttcaaggtgctg | 56 | 58 | 1 |
| 226941 | Coding | 4 | 111897 | gcctcctcggagagaagcca | 70 | 59 | 1 |
| 226942 | Coding | 4 | 111902 | gagctgcctcctcggagaga | 45 | 60 | 1 |
| 226943 | Coding | 4 | 111965 | accaagtctgtgatgatgtt | 68 | 61 | 1 |
| 226944 | Coding | 4 | 111970 | ggtagaccaagtctgtgatg | 44 | 62 | 1 |
| 226945 | Coding | 4 | 112028 | gcaaggtgcagggccatctc | 44 | 63 | 1 |
| 226946 | Coding | 11 | 5926 | tttccatggtcatccactgc | 74 | 64 | 1 |
| 226947 | Coding | 11 | 5931 | cagattttccatggtcatcc | 72 | 65 | 1 |
| 226948 | Coding | 4 | 113747 | acattattgacagcagctgc | 66 | 66 | 1 |
| 226949 | Coding | 4 | 115116 | gcctccttggcaaggttagg | 69 | 67 | 1 |
| 226950 | Coding | 4 | 115805 | aagccacactgggcaaacgg | 64 | 68 | 1 |
| 226951 | Coding | 4 | 115854 | atggtctgagctacctgccc | 69 | 69 | 1 |
| 226952 | Coding | 4 | 115890 | cccacagaggctgggaaagg | 78 | 70 | 1 |
| 226953 | Stop | 4 | 115895 | acttgcccacagaggctggg | 60 | 71 | 1 |
| | Codon | | | | | | |
| 226954 | 3′ UTR | 4 | 116067 | gtcacatctgaccagtcaga | 71 | 72 | 1 |
| 226955 | 3′ UTR | 4 | 116072 | tggtggtcacatctgaccag | 59 | 73 | 1 |
| 226956 | 3′ UTR | 4 | 116172 | ggactctctcacgcataaac | 68 | 74 | 1 |
| 226957 | 3′ UTR | 4 | 116695 | aaaggtcttgccctataaca | 44 | 75 | 1 |
| 226958 | 3′ UTR | 4 | 117121 | cattccaaacctttttctgg | 64 | 76 | 1 |
| 226959 | Intron | 4 | 118110 | cagacaaatcaggtaagtgg | 75 | 77 | 1 |
| 226960 | Intron: | 4 | 119090 | caaaacattacacctttggt | 65 | 78 | 1 |
| | exon | | | | | | |
| | junction | | | | | | |
| 226961 | Intron | 4 | 119806 | atttcacttaaggaatgtta | 45 | 79 | 1 |
| 226962 | Intron: | 4 | 19667 | tcaggagatcgagaccatcc | 41 | 80 | 1 |
| | exon | | | | | | |
| | junction | | | | | | |
| 226963 | Intron: | 4 | 34483 | agctccttacctggaaggca | 61 | 81 | 1 |
| | exon | | | | | | |
| | junction | | | | | | |
| 226964 | Intron: | 4 | 47121 | ccaaccactacgggtcttgg | 50 | 82 | 1 |
| | exon | | | | | | |
| | junction | | | | | | |
| 226965 | Intron: | 4 | 67672 | ccagtgaaacctttggaaag | 53 | 83 | 1 |
| | exon | | | | | | |
| | junction | | | | | | |
| 226966 | Intron | 4 | 82473 | atgttcttaccttgccagcc | 29 | 84 | 1 |
| 226967 | 5′ UTR | 4 | 83067 | ggcatactcactggcaaggc | 60 | 85 | 1 |
| 226968 | 5′ UTR | 4 | 106175 | aagcccttacttgcatgtct | 80 | 86 | 1 |
| 226969 | 5′ UTR | 4 | 108223 | gaatgacagagcaactgaag | 57 | 87 | 1 |
| 226970 | 5′ UTR | 12 | 8 | ctttctcgggtgtgcagccc | 16 | 88 | 1 |
| 226971 | 5′ UTR | 12 | 239 | gtatcttctcggtcgcctcc | 55 | 89 | 1 |
| 226972 | 5′ UTR | 12 | 1027 | cctgtctctttcccagagct | 37 | 90 | 1 |
| 226973 | 5′ UTR | 13 | 29 | cgccgccttgggcacccagg | 16 | 91 | 1 |

As shown in Table 1, SEQ ID NOs 14, 16, 17, 18, 19, 20, 22, 24, 26, 27, 29, 30, 31, 32, 33, 34, 35, 36, 37, 38, 39, 40, 41, 42, 44, 45, 46, 47, 48, 49, 50, 51, 52, 53, 54, 55, 57, 58, 59, 61, 64, 65, 66, 67, 68, 69, 70, 71, 72, 73, 74, 76, 77, 78, 81, 82, 83, 85, 86, 87 and 89 demonstrated at least 50% inhibition of human Notch2 expression in this assay and are therefore preferred. More preferred are SEQ ID Nos: 42, 51 and 86. The target regions to which these preferred sequences are complementary are herein referred to as “preferred target segments” and are therefore preferred for targeting by compounds of the present invention. These preferred target segments are shown in Table 2. These sequences are shown to contain thymine (T) but one of skill in the art will appreciate that thymine (T) is generally replaced by uracil (U) in RNA sequences. The sequences represent the reverse complement of the preferred antisense compounds shown in Table 1. “Target site” indicates the first (5′-most) nucleotide number on the particular target nucleic acid to which the oligonucleotide binds. Also shown in Table 2 is the species in which each of the preferred target segments was found.

TABLE 2

Sequence and position of preferred target segments
identified in Notch2.

| | | |
|---|---|---|
| TARGET | REV COMP | SEQ |

| | | | | |
|---|---|---|---|---|
| SEQ ID | TARGET | SEQUENCE | OF SEQ | ID |

| | | | | | | |
|---|---|---|---|---|---|---|
| SITEID | NO | SITE | (5′ to 3′) | ID | ACTIVE IN | NO |

| | | | | | | |
|---|---|---|---|---|---|---|
| 143548 | 4 | 25923 | gaatattgtcaacatcgaga | 14 | H. sapiens | 92 |
| 143550 | 4 | 34233 | ccagttctcctgcaaatgcc | 16 | H. sapiens | 93 |
| 143551 | 4 | 44403 | ccaggttttgaagggagcac | 17 | H. sapiens | 94 |
| 143552 | 4 | 63879 | ccttgtgagcatgcaggaaa | 18 | H. sapiens | 95 |
| 143553 | 4 | 63923 | ccactgtgagtgtctgaagg | 19 | H. sapiens | 96 |
| 143554 | 11 | 1455 | gtgcatgccaggtttcaaag | 20 | H. sapiens | 97 |
| 143556 | 4 | 65997 | atcgatcacccgaatggcta | 22 | H. sapiens | 98 |
| 143558 | 4 | 66007 | cgaatggctatgaatgccag | 24 | H. sapiens | 99 |
| 143560 | 4 | 66018 | gaatgccagtgtgccacagg | 26 | H. sapiens | 100 |
| 143561 | 4 | 67768 | tcctacacctgcatctgcaa | 27 | H. sapiens | 101 |
| 143563 | 4 | 71996 | gtgaaattaattttgatgac | 29 | H. sapiens | 102 |
| 143564 | 4 | 72001 | attaattttgatgactgtgc | 30 | H. sapiens | 103 |
| 143565 | 4 | 76276 | cattgatgagtgtgcctcca | 31 | H. sapiens | 104 |
| 143566 | 4 | 76376 | agctgctactcacaggtgaa | 32 | H. sapiens | 105 |
| 143567 | 4 | 76424 | ggaaactgtactggaggtct | 33 | H. sapiens | 106 |
| 143568 | 4 | 76429 | ctgtactggaggtctcagtg | 34 | H. sapiens | 107 |
| 143569 | 4 | 80699 | ctgaaccaaggaacctgctt | 35 | H. sapiens | 108 |
| 143570 | 4 | 82461 | ttgtgtgctcctggctggca | 36 | H. sapiens | 109 |
| 143571 | 11 | 2755 | tgccttgccaatccttgcca | 37 | H. sapiens | 110 |
| 143572 | 4 | 89815 | aagtgccagacagacatgaa | 38 | H. sapiens | 111 |
| 143573 | 11 | 3181 | gggaaaaactgtcagaccct | 39 | H. sapiens | 112 |
| 143574 | 11 | 3186 | aaactgtcagaccctggtga | 40 | H. sapiens | 113 |
| 143575 | 4 | 94030 | tgttgaacacttgtgccagc | 41 | H. sapiens | 114 |
| 143576 | 4 | 95897 | tatcagggtgtcaactgtga | 42 | H. sapiens | 115 |
| 143578 | 4 | 95907 | tcaactgtgagtatgaagtg | 44 | H. sapiens | 116 |
| 143579 | 4 | 102378 | ttgcctggctttgctgggga | 45 | H. sapiens | 117 |
| 143580 | 4 | 105700 | cagagcagctgtggacaagt | 46 | H. sapiens | 118 |
| 143581 | 4 | 106009 | catgcctgccagtgggatgg | 47 | H. sapiens | 119 |
| 143582 | 4 | 106099 | atcaacaaccagtgtgatga | 48 | H. sapiens | 120 |
| 143583 | 4 | 106135 | gagtgcctgtttgacaactt | 49 | H. sapiens | 121 |
| 143584 | 11 | 4519 | tgcaagtatgacaaatactg | 50 | H. sapiens | 122 |
| 143585 | 4 | 107505 | gtatgacaaatactgtgcag | 51 | H. sapiens | 123 |
| 143586 | 4 | 107510 | acaaatactgtgcagaccac | 52 | H. sapiens | 124 |
| 143587 | 4 | 107515 | tactgtgcagaccacttcaa | 53 | H. sapiens | 125 |
| 143588 | 4 | 107520 | tgcagaccacttcaaagaca | 54 | H. sapiens | 126 |
| 143589 | 4 | 107530 | ttcaaagacaaccactgtga | 55 | H. sapiens | 127 |
| 143591 | 4 | 111150 | cacagcagcaccttgaagct | 57 | H. sapiens | 128 |
| 143592 | 4 | 111155 | cagcaccttgaagctgcaga | 58 | H. sapiens | 129 |
| 143593 | 4 | 111897 | tggcttctctccgaggaggc | 59 | H. sapiens | 130 |
| 143595 | 4 | 111965 | aacatcatcacagacttggt | 61 | H. sapiens | 131 |
| 143598 | 11 | 5926 | gcagtggatgaccatggaaa | 64 | H. sapiens | 132 |
| 143599 | 11 | 5931 | ggatgaccatggaaaatctg | 65 | H. sapiens | 133 |
| 143600 | 4 | 113747 | gcagctgctgtcaataatgt | 66 | H. sapiens | 134 |
| 143601 | 4 | 115116 | cctaaccttgccaaggaggc | 67 | H. sapiens | 135 |
| 143602 | 4 | 115805 | ccgtttgcccagtgtggctt | 68 | H. sapiens | 136 |
| 143603 | 4 | 115854 | gggcaggtagctcagaccat | 69 | H. sapiens | 137 |
| 143604 | 4 | 115890 | cctttcccagcctctgtggg | 70 | H. sapiens | 138 |
| 143605 | 4 | 115895 | cccagcctctgtgggcaagt | 71 | H. sapiens | 139 |
| 143606 | 4 | 116067 | tctgactggtcagatgtgac | 72 | H. sapiens | 140 |
| 143607 | 4 | 116072 | ctggtcagatgtgaccacca | 73 | H. sapiens | 141 |
| 143608 | 4 | 116172 | gtttatgcgtgagagagtcc | 74 | H. sapiens | 142 |
| 143610 | 4 | 117121 | ccagaaaaaggtttggaatg | 76 | H. sapiens | 143 |
| 143611 | 4 | 118110 | ccacttacctgatttgtctg | 77 | H. sapiens | 144 |
| 143612 | 4 | 119090 | accaaaggtgtaatgttttg | 78 | H. sapiens | 145 |
| 143615 | 4 | 34483 | tgccttccaggtaaggagct | 81 | H. sapiens | 146 |
| 143616 | 4 | 47121 | ccaagacccgtagtggttgg | 82 | H. sapiens | 147 |
| 143617 | 4 | 67672 | ctttccaaaggtttcactgg | 83 | H. sapiens | 148 |
| 143619 | 4 | 83067 | gccttgccagtgagtatgcc | 85 | H. sapiens | 149 |
| 143620 | 4 | 106175 | agacatgcaagtaagggctt | 86 | H. sapiens | 150 |
| 143621 | 4 | 108223 | cttcagttgctctgtcattc | 87 | H. sapiens | 151 |
| 143623 | 12 | 239 | ggaggcgaccgagaagatac | 89 | H. sapiens | 152 |

As these “preferred target segments” have been found by experimentation to be open to, and accessible for, hybridization with the antisense compounds of the present invention, one of skill in the art will recognize or be able to ascertain, using no more than routine experimentation, further embodiments of the invention that encompass other compounds that specifically hybridize to these preferred target segments and consequently inhibit the expression of Notch2.

According to the present invention, antisense compounds include antisense oligomeric compounds, antisense oligonucleotides, siRNAs, external guide sequence (EGS) oligonucleotides, alternate splicers, and other short oligomeric compounds which hybridize to at least a portion of the target nucleic acid.

### Example 16

Western Blot Analysis of Notch2 Protein Levels

Western blot analysis (immunoblot analysis) is carried out using standard methods. Cells are harvested 16-20 h after oligonucleotide treatment, washed once with PBS, suspended in Laemmli buffer (100 ul/well), boiled for 5 minutes and loaded on a 16% SDS-PAGE gel. Gels are run for 1.5 hours at 150 V, and transferred to membrane for western blotting. Appropriate primary antibody directed to Notch2 is used, with a radiolabeled or fluorescently labeled secondary antibody directed against the primary antibody species. Bands are visualized using a PHOSPHORIMAGER™ (Molecular Dynamics, Sunnyvale Calif.).

1. An antisense compound 8 to 80 nucleobases in length targeted to a nucleic acid molecule encoding Notch2, wherein said compound is at least 70% complementary to said nucleic acid molecule encoding Notch2, and wherein said compound inhibits the expression of Notch2 mRNA by at least 10%.

2. The antisense compound of claim 1 comprising 12 to 50 nucleobases in length.

3. The antisense compound of claim 2 comprising 15 to 30 nucleobases in length.

4. The antisense compound of claim 1 comprising an oligonucleotide.

5. The antisense compound of claim 4 comprising a DNA oligonucleotide.

6. The antisense compound of claim 4 comprising an RNA oligonucleotide.

7. The antisense compound of claim 4 comprising a chimeric oligonucleotide.

8. The antisense compound of claim 4 wherein at least a portion of said compound hybridizes with RNA to form an oligonucleotide-RNA duplex.

9. The antisense compound of claim 1 having at least 80% complementarity with said nucleic acid molecule encoding Notch2.

10. The antisense compound of claim 1 having at least 90% complementarity with said nucleic acid molecule encoding Notch2.

11. The antisense compound of claim 1 having at least 95% complementarity with said nucleic acid molecule encoding Notch2.

12. The antisense compound of claim 1 having at least 99% complementarity with said nucleic acid molecule encoding Notch2.

13. The antisense compound of claim 1 having at least one modified internucleoside linkage, sugar moiety, or nucleobase.

14. The antisense compound of claim 1 having at least one 2′-O-methoxyethyl sugar moiety.

15. The antisense compound of claim 1 having at least one phosphorothioate internucleoside linkage.

16. The antisense compound of claim 1 wherein at least one cytosine is a 5-methylcytosine.

17. A method of inhibiting the expression of Notch2 in a cell or tissue comprising contacting said cell or tissue with the antisense compound of claim 1 so that expression of Notch2 is inhibited.

18. A method of screening for a modulator of Notch2, the method comprising the steps of:
contacting a preferred target segment of a nucleic acid molecule encoding Notch2 with one or more candidate modulators of Notch2, and
identifying one or more modulators of Notch2 expression which modulate the expression of Notch2.

19. The method of claim 18 wherein the modulator of Notch2 expression comprises an oligonucleotide, an antisense oligonucleotide, a DNA oligonucleotide, an RNA oligonucleotide, an RNA oligonucleotide having at least a portion of said RNA oligonucleotide capable of hybridizing with RNA to form an oligonucleotide-RNA duplex, or a chimeric oligonucleotide.

20. A diagnostic method for identifying a disease state comprising identifying the presence of Notch2 in a sample using at least one of the primers comprising SEQ ID NOs 5 or 6, or the probe comprising SEQ ID NO: 7.

21. A kit or assay device comprising the antisense compound of claim 1.

22. A method of treating an animal having a disease or condition associated with Notch2 comprising administering to said animal a therapeutically or prophylactically effective amount of the antisense compound of claim 1 so that expression of Notch2 is inhibited.

23. The method of claim 22 wherein the disease or condition is an autoimmune disorder.

24. The antisense compound of claim 1, wherein said antisense compound comprises at least an 8-nucleobase portion of SEQ ID NOs 14, 16, 17, 18, 19, 20, 22, 24, 26, 27, 29, 30, 31, 32, 33, 34, 35, 36, 37, 38, 39, 40, 41, 42, 44, 45, 46, 47, 48, 49, 50, 51, 52, 53, 54, 55, 57, 58, 59, 61, 64, 65, 66, 67, 68, 69, 70, 71, 72, 73, 74, 76, 77, 78, 81, 82, 83, 85, 86, 87 or 89.

25. The antisense compound of claim 24, wherein said antisense compound has a sequence selected from the group consisting of SEQ ID NOs 14, 16, 17, 18, 19, 20, 22, 24, 26, 27, 29, 30, 31, 32, 33, 34, 35, 36, 37, 38, 39, 40, 41, 42, 44, 45, 46, 47, 48, 49, 50, 51, 52, 53, 54, 55, 57, 58, 59, 61, 64, 65, 66, 67, 68, 69, 70, 71, 72, 73, 74, 76, 77, 78, 81, 82, 83, 85, 86, 87 and 89.

26. The antisense compound of claim 1, wherein said antisense compound comprises an antisense nucleic acid molecule that is specifically hybridizable with a 5′-untranslated region (5′UTR) of a nucleic acid molecule encoding Notch2.

27. The antisense compound of claim 1, wherein said antisense compound comprises an antisense nucleic acid molecule that is specifically hybridizable with a start region of a nucleic acid molecule encoding Notch2.

28. The antisense compound of claim 1, wherein said antisense compound comprises an antisense nucleic acid molecule that is specifically hybridizable with a coding region of a nucleic acid molecule encoding Notch2.

29. The antisense compound of claim 1, wherein said antisense compound comprises an antisense nucleic acid molecule that is specifically hybridizable with a stop region of a nucleic acid molecule encoding Notch2.

30. The antisense compound of claim 1, wherein said antisense compound comprises an antisense nucleic acid molecule that is specifically hybridizable with a 3′-untranslated region of a nucleic acid molecule encoding Notch2.

31. The antisense compound of claim 1 which is single-stranded.
